(19) United States
(12) Patent Application Publication
(10) Pub. No.: US 2013/0267474 A1
(43) Pub. Date: Oct. 10, 2013

(54) NOVEL PHYTOCHEMICALS FROM EXTRACTS OF MAPLE SYRUPS AND MAPLE TREES AND USES THEREOF

(71) Applicants: Navindra P. Seeram, Charlestown, RI (US); Liya Li, Wakefield, RI (US); Geneviéve Béland, St-Hyacinthe (CA); Julie Barbeau, Boucherville (CA)

(72) Inventors: Navindra P. Seeram, Charlestown, RI (US); Liya Li, Wakefield, RI (US); Geneviéve Béland, St-Hyacinthe (CA); Julie Barbeau, Boucherville (CA)

(73) Assignees: UNIVERSITY OF RHODE ISLAND, Kingston, RI (US); FÉDÉRATION DES PRODUCTEURS ACÉRICOLES DU QUÉBEC, Longueuil, QC (CA)

(21) Appl. No.: 13/817,732

(22) PCT Filed: Aug. 19, 2011
(86) PCT No.: PCT/CA2011/000943

§ 371 Date: Jun. 11, 2013

Related U.S. Application Data
(60) Provisional application No. 61/375,441, filed on Aug. 20, 2010.
(60) Provisional application No. 61/405,819, filed on Oct. 22, 2010.
(60) Provisional application No. 61/405,812, filed on Oct. 22, 2010.
(60) Provisional application No. 61/446,678, filed on Feb. 25, 2011.
(60) Provisional application No. 61/468,790, filed on Mar. 29, 2011.
(60) Provisional application No. 61/493,532, filed on Jun. 6, 2011.

(51) Int. Cl. C07H 15/207; A23L 1/30; C07C 43/23; C07D 307/33; C07C 49/337

(52) U.S. Cl. CPC C07H 15/207; C07D 307/33; C07C 49/337; C07C 43/23; A23L 1/3002; A23L 1/3004
USPC 514/25; 514/473; 514/689; 514/721; 536/4.1; 549/313; 568/337; 568/660

(57) ABSTRACT

The present invention describes phytochemicals present in maple syrup and maple tree extracts by butanol, ethyl acetate and methanol. Novel compounds are isolated from maple syrups, including one compound Quebecol generated in the maple syrup manufacturing process. Also described are digesting extract of maple syrup. The phytochemicals may be used for the treatment or prevention of cancers, metabolic syndromes, diabetes, microorganism infections and/or antioxidants.

### CROSS-REFERENCE TO RELATED APPLICATIONS

This application claims priority from U.S. provisional patent applications 61/375,441, filed Aug. 20; 2010, 61/405,812, filed Oct. 22, 2010; 61/405,819, filed Oct. 22, 2010; 61/446,678, filed Feb. 25, 2011; 61/468,790, filed Mar. 29, 2011; and 61/493,532, filed Jun. 6, 2011, the specifications of which is hereby incorporated by reference.

### BACKGROUND

(a) Field

The subject matter disclosed generally relates to novel phytochemicals, novel maple syrup phytochemicals, a method of isolating these phytochemicals and method of uses thereof.

(b) Related Prior Art

Maple syrup (MS) is a natural sweetener obtained by concentrating the sap collected from certain maple species including the sugar maple (Acer saccharum) which is native to North America. MS is primarily produced in north eastern North America and the vast majority of the world's supply comes from Canada (85%; primarily Quebec), followed by the United States (15%; primarily New England/New York region). Indeed, MS production is among the few agricultural processes that is native to North America and not introduced by early settlers. Further, MS is the largest commercially available food product consumed by humans which is derived totally from the sap of deciduous trees.

MS is produced by thermal evaporation of the colorless watery sap collected from maple trees in late winter to early spring. Because of its high water content, about 40 L of sap is required to produce 1 L of MS. During the concentration process of transforming sap to syrup, the characteristic flavor, color, and odor of MS develops. Typically, the color of the syrup becomes darker as the season progresses, and based on Canadian standards, MS is graded as extra light (grade AA), light (grade A), medium/amber (grade B), and dark (grade C).

Being a plant-derived natural product, it is not surprising that MS contains phytochemicals (naturally present in the xylem sap), as well as process-derived compounds (formed during thermal evaporation of sap). Apart from sucrose, which is its dominant sugar, MS contains organic acids, amino acids, minerals, and lignin derived flavor compounds. Among the phytochemicals which have been previously reported from MS, the phenolic class predominates. For example, vanillin, syringaldehyde, coniferaldehyde, cinnamic acid and benzoic acid derivatives, flavanols, and flavonols have been identified in MS extracts.

The presence of a diverse range of phenolic sub-classes in MS is interesting given that this large class of dietary phytochemicals has attracted significant research attention due to their diverse biological functions and potential positive effects on human health. Recently, phenolic-enriched extracts of MS or extracts of maple trees (from the sap, the samara (including the fruits, the seeds as well as the stem), leaves (including the stem), twigs, roots, heartwood and sap wood, and bark of any Acer tree) were shown to have antioxidant, antimutagenic, and human cancer cell antiproliferative properties. While the phenolic constituents in several organic solvent extracts, namely, ethyl acetate, chloroform, dichloromethane and diethyl ether of MS have been investigated, constituents in a MS butanol extract are yet to be reported.

MS is popularly consumed worldwide and its production is of significant cultural and economical importance to north eastern North America. Therefore, increased knowledge of the chemical constituents of MS would aid in the authentication, characterization, and subsequent detection of intentional adulteration of this premium natural sweetener. Also, characterization of the different chemical sub-classes of bioactive phenolics, and ascertaining their levels, would aid in evaluating the potential human health benefits of MS consumption.

### SUMMARY

According to an embodiment, there is provided a molecule consisting of:

5-(3″,4″-dimethoxyphenyl)-3-hydroxy-3-(4′-hydroxy-3′-methoxybenzyl)-4-hydroxymethyl-dihydrofuran-2-one

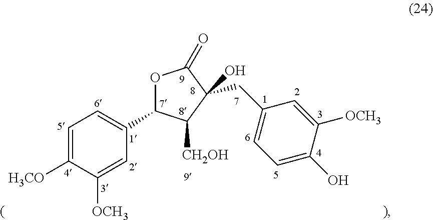 [SMILES: COc1cc(C[C@@]2(O)C(=O)O[C@@H](c3ccc(OC)c(OC)c3)[C@@H]2CO)ccc1O]

(erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol

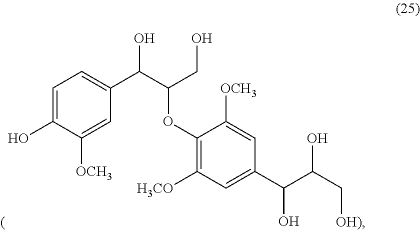

(erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol

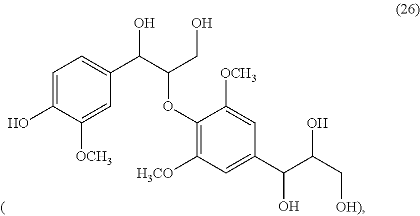

2,3-dihydroxy-1-(3,4-dihydroxyphenyl)-1-propanone

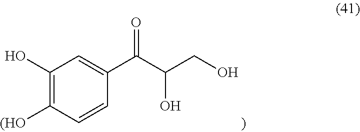

Quebecol

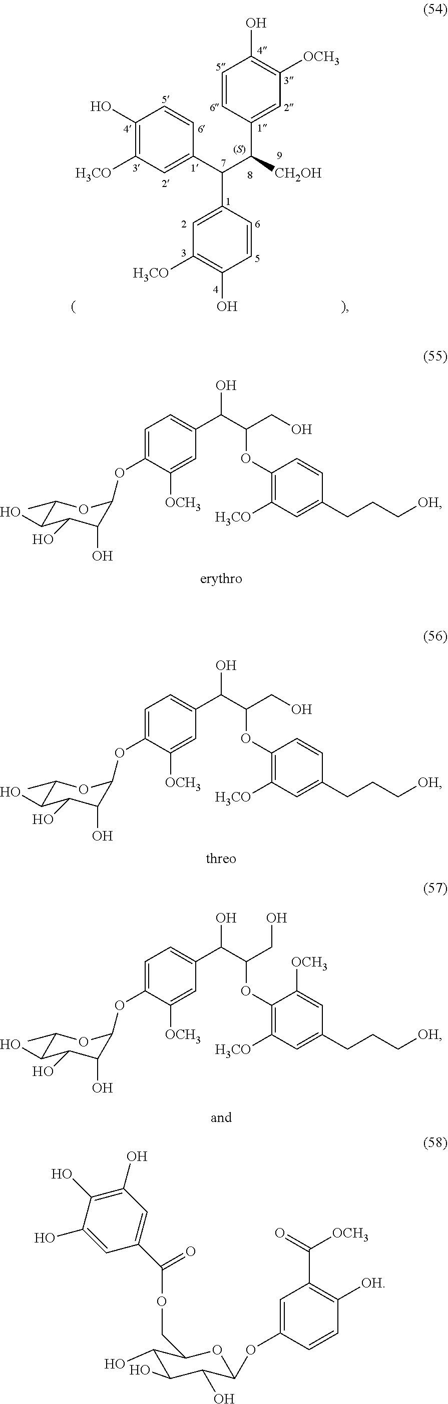

According to another embodiment, there is provided a phytochemical present in a maple tree butanol extract, ethyl acetate extract, and methanol extract, which comprises a molecule chosen from:
Lyoniresinol,
Isolariciresinol,
secoisolariciresinol,
Dehydroconiferyl alcohol,
5′-methoxy-dehydroconiferyl alcohol,
erythro-guaiacylglycerol-β-O-4′-coniferyl alcohol,
erythro-guaiacylglycerol-β-O-4′-dihydroconiferyl alcohol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
5-(3″,4″-dimethoxyphenyl)-3-hydroxy-3-(4′-hydroxy-3′-methoxybenzyl)-4-hydroxymethyl-dihydrofuran-2-one,
Scopoletin,
Fraxetin,
Isofraxidin,
Gallic acid,
Ginnalin A (acertannin),
Syringic acid,
Ginnalin B,
Ginnalin C,
Trimethyl gallic acid methyl ester
(E)-3,3′-dimethoxy-4,4′-dihydroxy stilbene,
p-coumaric acid,
Ferulic acid,
(E)-Coniferol,
Syringenin,
Dihydroconiferyl alcohol,
C-Veratroylglycol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
3′,4′,5′-Trihydroxyacetophenone,
4-Acetylcatechol,
2,4,5-Trihydroxyacetophenone,
1-(2,3,4-trihydroxy-5-methylphenyl)-ethanone,
2-Hydroxy-3′,4′-dihydroxyacetophenone,
Vanillin,
Syringaldehyde,
Catechaldehyde,
3,4-Dihydroxy-2-methylbenzaldehyde,
Catechol,
Catechin,
Epicatechin,
Quebecol,
(erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo,erythro)-1-[4-[(2-hydroxy-2-(4-hydroxy-3-methoxyphenyl)-1-(hydroxymethyl)ethoxy]-3-methoxyphenyl]-1,2,3-propanetriol,
(threo,threo)-1-[4-[(2-hydroxy-2-(4-hydroxy-3-methoxyphenyl)-1-(hydroxymethyl)ethoxy]-3-methoxyphenyl]-1,2,3-propanetriol,
threo-guaiacylglycerol-β-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Syringaresinol,
Icariside E4,
Sakuraresinol,
1,2-diguaiacyl-1,3-propanediol
protocatechuic acid,
4-(dimethoxymethyl)-pyrocatechol,
Tyrosol,
4-hydroxycatechol, and
Phaseic acid.

The phytochemical may be from a maple tree butanol extract, which comprises a molecule chosen from:
Lyoniresinol,
Secoisolariciresinol,
Dehydroconiferyl alcohol,
5′-methoxydehydroconiferyl alcohol,
(1,3-Propanediol, 1-(4-hydroxy-3-methoxyphenyl)-2-[4-[(1E)-3-hydroxy-1-propenyl]-2-methoxyphenoxy]-, (1R,2R)),
1-(4-hydroxy-3-methoxyphenyl)-2-[4-(3-hydroxypropyl)-2-methoxyphenoxy]-propane-1,3-diol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
Scopoletin,
Fraxetin,
(E)-3,3′-dimethoxy-4,4′-dihydroxystilbene,
2-hydroxy-3′,4′-dihydroxyacetophenone,
1-(2,3,4-trihydroxy-5-methylphenyl)-ethanone,
2,4,5-trihydroxyacetophenone,
Catechaldehyde,
Vanillin,
Syringaldehyde,
Gallic acid,
Trimethyl gallic acid methyl ester,
Syringic acid,
Syringenin,
(E)-coniferol,
C-veratroylglycol,
Catechol,
Quebecol,
Catechin, and
Epicatechin.

The phytochemical may be from a maple tree ethyl acetate extract, which comprises a molecule chosen from:
Lyoniresinol,
Secoisolariciresinol,
1-(4-hydroxy-3-methoxyphenyl)-2-[4-(3-hydroxypropyl)-2 methoxyphenoxy]-propane-1,3-diol,
Scopoletin,
C-veratroylglycol,
5-(3″,4″-dimethoxyphenyl)-3-hydroxy-3-(4′-hydroxy-3′-methoxybenzyl)-4-hydroxymethyl-dihydrofuran-2-one,
(erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo,erythro)-1-[4-[(2-hydroxy-2-(4-hydroxy-3-methoxyphenyl)-1-(hydroxymethyl)ethoxy]-3-methoxyphenyl]-1,2,3-propanetriol,
(threo,threo)-1-[4-[(2-hydroxy-2-(4-hydroxy-3-methoxyphenyl)-1-(hydroxymethyl)ethoxy]-3-methoxyphenyl]-1,2,3-propanetriol,
threo-guaiacylglycerol-β-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Isolariciresinol,
Syringaresinol,
Icariside E4,
Sakuraresinol,
1,2-diguaiacyl-1,3-propanediol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
Dihydroconiferyl alcohol,
4-Acetylcatechol,
3′,4′,5′-Trihydroxyacetophenone,
3,4-Dihydroxy-2-methylbenzaldehyde,
Protocatechuic acid,
4-(dimethoxymethyl)-pyrocatechol,
Tyrosol,
Isofraxin,
4-hydroxycatechol, and
Phaseic acid.

The phytochemical may be from a maple tree methanol extract, which comprises a molecule chosen from:
Gallic acid,
(E)-3,3′-dimethoxy-4,4′-dihydroxy stilbene,
Syringic acid,
C-veratroylglycol,
1-(4-hydroxy-3-methoxyphenyl)-2-[4-(3-hydroxypropyl)-2-methoxyphenoxy]-propane-1,3-diol (guaiacylglycerol-β-O-4′-dihydroconiferyl alcohol),
3-[(4-[(6-dexoy-α-L-mannopyranosyl)oxy]-3-methoxyphenyl)-5-(3,4-dimethoxyphenyl)dihydro-3-hydroxy-4-(hydroxymethyl)-2(3H)-furanone,
Lyoniresinol,
2-Hydroxy-3′,4′-dihydroxyacetophenone,
Syringenin,
Catechol,
Syringaldehyde,
Vanillin,
1,3-propanediol, 1-(4-hydroxy-3-methoxyphenyl)-2-[4-[(1E)-3-hydroxy-1-propenyl]-2-methoxyphenoxy]-,(1R,2R),
2,3-dihydro-3-(hydroxymethyl)-2-(4-hydroxy-3-methoxyphenyl)-7-methoxy-5-benzofuranpropanol (dihydrodehydrodiconiferyl alcohol),
Ferulic acid,
Catechaldehyde,
Fraxetin,
(E)-coniferyl alcohol (coniferol),
Scopoletin,
1-(2,3,4-trihydroxy-5-methylphenyl)-ethanone,
p-coumaric acid,
Secoisolariciresinol,
Catechin,
Epicatechin,
3′,4′,5′-Trihydroxyacetophenone,
4-(dimethoxymethyl)-pyrocatechol,
4-acetylcatechol,
2,3-dihydroxy-1-(3,4-dihydroxyphenyl)-1-propanone,
Dihydroconiferyl alcohol,
Isofraxidin,
2,3-dihydroxy-1-(4-hydroxy-3,5-dimethoxyphenyl)-1-propanone,
Tyrosol,
3-hydroxy-1-(4-hydroxy-3,5-dimethoxyphenyl)propan-1-one,
Isolariciresinol,
5-(3″,4″-dimethoxyphenyl)-3-hydroxy-3-(4′-hydroxy-3′-methoxybenzyl)-4-hydroxymethyl-dihydrofuran-2-one,
Protocatechuic acid,
Threo-guaiacylglycerol-β-O-4′-dihydroconiferyl alcohol,
4-hydroxycatechol,
(erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
1,2-diguaiacyl-1,3-propanediol,
1-[4-[(1R,2R)-2-hydroxy-2-(4-hydroxy-3-methoxyphenyl)-1-(hydroxymethyl)ethoxy]-3-methoxyphenyl]-1,2,3-propanetriol,
Leptolepisol D,
Sakuraresinol,
(erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol,
Icariside E4,
Syringaresinol,
Acernikol,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
2-[4-[(2S,3R)-2,3-dihydro-3-(hydroxymethyl)-5-(3-hydroxy propyl)-7-methoxy-2-benzofuranyl]-2,6-dimethoxyphenoxy]-1-(4-hydroxy-3-methoxyphenyl)-1,3-propanediol, and
Buddenol E.

According to another embodiment, there is disclosed a composition comprising a molecule according to the present invention, at least one phytochemical according to the present invention, or combinations thereof.

The composition may be a cosmeceutical composition, a cosmetic composition, a nutraceutical composition, a functional food, a food ingredient, an additive, a non-food ingredient, a cosmeto-food, a pharmaceutical, and a food supplement, a natural health product, or combinations thereof.

According to another embodiment, there is provided a method to prevent micro-organism infection, kill or inhibit bacteria or treat micro-organism infection in a subject, which comprises administering an antimicro-organism amount of a molecule according to the present invention.

According to another embodiment, there is provided a method to prevent micro-organism infection, kill or inhibit bacteria or treat micro-organism infection in a subject, which comprises administering an antimicro-organism amount of at least one phytochemical according to the present invention.

According to another embodiment, there is provided a method to inhibit tumor growth in a subject, which comprises administering an anticancer amount of a molecule according to the present invention.

According to another embodiment, there is provided a method to inhibit tumor growth in a subject, which comprises administering an anticancer amount of a phytochemical according to the present invention.

According to another embodiment, there is provided a method of treating a disease in a subject, which comprises administering a therapeutically effective amount of a molecule according to the present invention.

According to another embodiment, there is provided a method of treating or preventing a disease in a subject, which comprises administering a therapeutically effective amount of a phytochemical according to the present invention

The disease may be chosen from a metabolic syndrome, a diabetes, a neurodegenerative disease, an oxidative stress related disease, an intestinal dysfunction, a heart disease, inflammation and an inflammatory condition.

The intestinal dysfunction may be chosen from an inflammatory bowel disease, Crohn's disease, an ulcerative colitis, collagenous colitis, lymphocytic colitis, ischaemic colitis, diversion colitis, Behçet's disease, and indeterminate colitis.

According to another embodiment, there is provided a use of a molecule according to the present invention for the preparation of a medicament for the treatment of a disease.

According to another embodiment, there is provided a use of a molecule according to the present invention for the treatment of a disease.

According to another embodiment, there is provided a use of a molecule according to the present invention as an antioxidant.

According to another embodiment, there is provided a use of a phytochemical according to the present invention for the preparation of a medicament for the treatment of a disease.

According to another embodiment, there is provided a use of a phytochemical according to the present invention for the treatment of a disease.

According to another embodiment, there is provided a use of a phytochemical according to the present invention as an antioxidant.

The disease may be chosen from a metabolic syndrome, a diabetes, a neurodegenerative disease, an oxidative stress related disease, an intestinal dysfunction, a heart disease, inflammation and an inflammatory condition.

The intestinal dysfunction may be chosen from an inflammatory bowel disease, Crohn's disease, an ulcerative colitis, collagenous colitis, lymphocytic colitis, ischaemic colitis, diversion colitis, Behçet's disease, and indeterminate colitis.

According to another embodiment, there is provided a process of preparing a maple syrup digested extract, comprising treating said maple syrup with a gastrointestinal enzyme for a time sufficient to digest a phenolic content of said maple syrup.

The gastrointestinal enzyme may be chosen from pepsin-HCl (pH 2.0), pancreatin and bile salts (pH 6.5), or combinations thereof.

The treating may be with pepsin-HCl (pH 2.0) for 2 h followed by pancreatin and bile salts (pH 6.5) for 2 h.

According to another embodiment, there is provided an enzyme digested extract obtained by the process of the present invention.

According to another embodiment, there is provided a method to inhibit tumor growth in a subject, which comprises administering an anticancer amount of an extract according to the present invention.

According to another embodiment, there is provided a method of treating of preventing a disease comprising administering to a subject in need thereof a therapeutically effective amount of an extract according to the present invention.

The disease may be chosen from a metabolic syndrome, a diabetes, arthritis, a neurodegenerative disease, an inflammation, an inflammatory condition, an oxidative stress related disease, intestinal dysfunction and heart disease.

The intestinal dysfunction may be chosen from an inflammatory bowel disease, Crohn's disease, an ulcerative colitis, collagenous colitis, lymphocytic colitis, ischaemic colitis, diversion colitis, Behçet's disease, and indeterminate colitis.

The extract may be a cosmeceutical composition, a cosmetic composition, a nutraceutical composition, a functional food, a food ingredient, an additive, a non-food ingredient, a cosmeto-food, a pharmaceutical, and a food supplement, a natural health product, or combinations thereof.

According to another embodiment, there is provided a use of an extract according to the present invention for the preparation of a medicament for the treatment of a disease.

According to another embodiment, there is provided a use of an extract according to the present invention for the treatment of a disease.

The disease may be chosen from a metabolic syndrome, a diabetes, arthritis, a neurodegenerative disease, an inflammation, an inflammatory condition, an oxidative stress related disease, intestinal dysfunction and heart disease.

The intestinal dysfunction may be chosen from an inflammatory bowel disease, Crohn's disease, an ulcerative colitis, collagenous colitis, lymphocytic colitis, ischaemic colitis, diversion colitis, Behçet's disease, and indeterminate colitis.

According to another embodiment, there is provided a use of an extract according to the present invention as an antioxidant.

According to another embodiment, there is provided a method of inhibiting an α-glucosidase in a subject which comprises administering an inhibiting amount of a maple tree extract comprising at least one phytochemical.

According to another embodiment, there is provided a method of inhibiting or preventing an inflammation and an inflammatory condition in a subject which comprises administering an inhibiting amount of a maple tree extract comprising at least one phytochemical.

According to another embodiment, there is provided a method of treating or preventing a disease in a subject which comprises administering a therapeutically effective amount of a maple tree extract comprising at least one phytochemical.

The disease may be chosen from a cancer, a metabolic syndrome, a diabetes, a neurodegenerative disease, an oxidative stress related disease, an intestinal dysfunction, a heart disease, an inflammation and an inflammatory condition.

The intestinal dysfunction may be chosen from an inflammatory bowel disease, Crohn's disease, an ulcerative colitis, collagenous colitis, lymphocytic colitis, ischaemic colitis, diversion colitis, Behçet's disease, and indeterminate colitis.

The maple tree extract may be at least one of

a butanol extract from a maple syrup,

an ethyl acetate extract from a maple syrup,

a methanol extract from a maple syrup,

a methanol extract from a sugar maple leaf,

a methanol extract from a red maple leaf,

a methanol extract from a red maple stem,

a methanol extract from a sugar maple bark,

a methanol extract from a red maple bark,

a methanol extract from a red maple fruit,

a methanol extract from a red maple heartwood,

a methanol extract from a sugar maple heartwood,

an ethyl acetate extract from a sugar maple bark, and

a butanol extract from a sugar maple bark.

The at least one phytochemical may be from a butanol extract from a maple syrup which comprises a molecule chosen from
Lyoniresinol,
Secoisolariciresinol,
Dehydroconiferyl alcohol,
5′-methoxydehydroconiferyl alcohol,
(1,3-Propanediol, 1-(4-hydroxy-3-methoxyphenyl)-2-[4-[(1E)-3-hydroxy-1-propenyl]-2-methoxyphenoxy]-, (1R,2R)),
1-(4-hydroxy-3-methoxyphenyl)-2-[4-(3-hydroxypropyl)-2-methoxyphenoxy]-propane-1,3-diol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
Scopoletin,
Fraxetin,
(E)-3,3′-dimethoxy-4,4′-dihydroxystilbene,
2-hydroxy-3′,4′-dihydroxyacetophenone,
1-(2,3,4-trihydroxy-5-methylphenyl)-ethanone,
2,4,5-trihydroxyacetophenone,
Catechaldehyde,
Vanillin,
Syringaldehyde,
Gallic acid,
Trimethyl gallic acid methyl ester,
Syringic acid,
Syringenin,
(E)-coniferol,
C-veratroylglycol,
Catechol,
Quebecol,
Catechin, and
Epicatechin.

The at least one phytochemical is from an ethyl acetate extract from a maple syrup which comprises a molecule chosen from:
Lyoniresinol,
Secoisolariciresinol,
1-(4-hydroxy-3-methoxyphenyl)-2-[4-(3-hydroxypropyl)-2 methoxyphenoxy]-propane-1,3-diol,
Scopoletin,
C-veratroylglycol,
5-(3″,4″-dimethoxyphenyl)-3-hydroxy-3-(4′-hydroxy-3′-methoxybenzyl)-4-hydroxymethyl-dihydrofuran-2-one,
(erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo,erythro)-1-[4-[(2-hydroxy-2-(4-hydroxy-3-methoxyphenyl)-1-(hydroxymethyl)ethoxy]-3-methoxyphenyl]-1,2,3-propanetriol,
(threo,threo)-1-[4-[(2-hydroxy-2-(4-hydroxy-3-methoxyphenyl)-1-(hydroxymethyl)ethoxy]-3-methoxyphenyl]-1,2,3-Propanetriol,
threo-guaiacylglycerol-3-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Isolariciresinol,
Syringaresinol,
Icariside E4,
Sakuraresinol,
1,2-diguaiacyl-1,3-propanediol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
Dihydroconiferyl alcohol,
4-Acetylcatechol,
3′,4′,5′-Trihydroxyacetophenone,
3,4-Dihydroxy-2-methylbenzaldehyde,
Protocatechuic acid,
4-(dimethoxymethyl)-pyrocatechol,
Tyrosol,
Isofraxin,
4-hydroxycatechol, and
Phaseic acid.

The at least one phytochemical may be from a methanol extract from maple syrup which comprises a molecule chosen from:
Gallic acid,
(E)-3,3′-dimethoxy-4,4′-dihydroxy stilbene,
Syringic acid,
C-veratroylglycol,
1-(4-hydroxy-3-methoxyphenyl)-2-[4-(3-hydroxypropyl)-2-methoxyphenoxy]-propane-1,3-diol (guaiacylglycerol-β-O-4′-dihydroconiferyl alcohol),
3-[(4-[(6-dexoy-α-L-mannopyranosyl)oxy]-3-methoxyphenyl)-5-(3,4-dimethoxyphenyl)dihydro-3-hydroxy-4-(hydroxymethyl)-2(3H)-furanone,
Lyoniresinol,
2-Hydroxy-3′,4′-dihydroxyacetophenone,
Syringenin,
Catechol,
Syringaldehyde,
Vanillin,
1,3-propanediol, 1-(4-hydroxy-3-methoxyphenyl)-2-[4-[(1E)-3-hydroxy-1-propenyl]-2-methoxyphenoxy]-,(1R,2R),
2,3-dihydro-3-(hydroxymethyl)-2-(4-hydroxy-3-methoxyphenyl)-7-methoxy-5-benzofuranpropanol (dihydrodehydrodiconiferyl alcohol),
Ferulic acid,
Catechaldehyde,
Fraxetin,
(E)-coniferyl alcohol (coniferol),
Scopoletin,
1-(2,3,4-trihydroxy-5-methylphenyl)-ethanone,
p-coumaric acid,
Secoisolariciresinol,
Catechin,
Epicatechin,
3′,4′,5′-Trihydroxyacetophenone,
4-(dimethoxymethyl)-pyrocatechol,
4-acetylcatechol,
2,3-dihydroxy-1-(3,4-dihydroxyphenyl)-1-propanone,
Dihydroconiferyl alcohol,
Isofraxidin,
2,3-dihydroxy-1-(4-hydroxy-3,5-dimethoxyphenyl)-1-propanone,
Tyrosol,
3-hydroxy-1-(4-hydroxy-3,5-dimethoxyphenyl)propan-1-one,
Isolariciresinol,
5-(3″,4″-dimethoxyphenyl)-3-hydroxy-3-(4′-hydroxy-3′-methoxybenzyl)-4-hydroxymethyl-dihydrofuran-2-one,
Protocatechuic acid,
Threo-guaiacylglycerol-β-O-4′-dihydroconiferyl alcohol,
4-hydroxycatechol,
(erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
1,2-diguaiacyl-1,3-propanediol,
(threo,erythro) 1-[4-[(1R,2R)-2-hydroxy-2-(4-hydroxy-3-methoxyphenyl)-1-(hydroxymethyl)ethoxy]-3-methoxyphenyl]-1,2,3-propanetriol,
(threo,threo) 1-[4-[(1R,2R)-2-hydroxy-2-(4-hydroxy-3-methoxyphenyl)-1-(hydroxymethyl)ethoxy]-3-methoxyphenyl]-1,2,3-propanetriol,
Leptolepisol D,
Sakuraresinol,
(erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol,
Icariside E4,
Syringaresinol,
Acernikol,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
2-[4-[(2S,3R)-2,3-dihydro-3-(hydroxymethyl)-5-(3-hydroxy propyl)-7-methoxy-2-benzofuranyl]-2,6-dimethoxyphenoxy]-1-(4-hydroxy-3-methoxyphenyl)-1,3-propanediol, and
Buddenol E.

The at least one phytochemical may be from a methanol extract from a red maple bark which comprises a molecule chosen from:

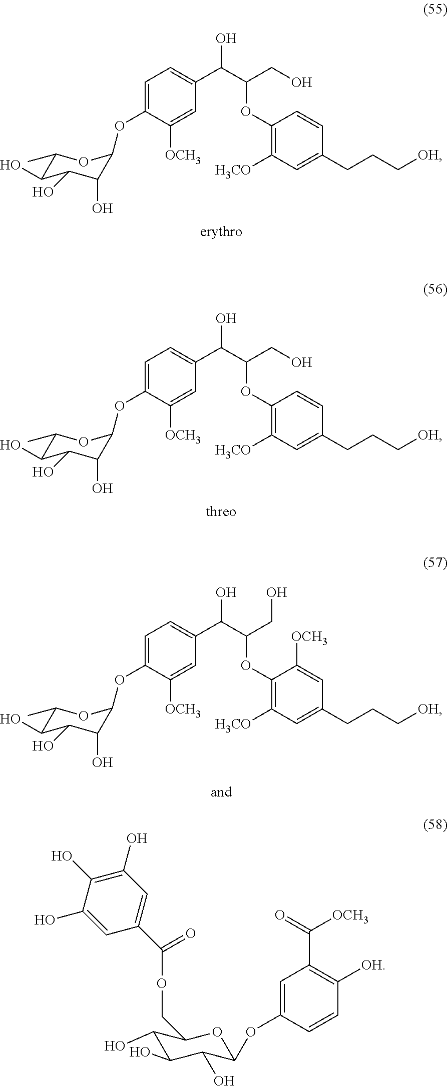 [SMILES: COc1cc(CCCO)ccc1OC(CO)C(O)c1ccc(OC2OC(C)C(O)C(O)C2O)c(OC)c1 ; COc1cc(CCCO)ccc1OC(CO)C(O)c1ccc(OC2OC(C)C(O)C(O)C2O)c(OC)c1 ; COc1cc(C(O)C(CO)Oc2c(OC)cc(CCCO)cc2OC)ccc1OC1OC(C)C(O)C(O)C1O ; COC(=O)c1cc(OC2OC(COC(=O)c3cc(O)c(O)c(O)c3)C(O)C(O)C2O)ccc1O]

The inhibition of α-glucosidase may be for the treatment of a diabetes, and the diabetes may be type 2 diabetes.

According to another embodiment, there is provided a use of a maple tree extract comprising at least one phytochemical according to the present invention for the preparation of a medicament for the inhibition of an α-glucosidase.

According to another embodiment, there is provided a use of a maple tree extract comprising at least one phytochemical according to the present invention for the preparation of a medicament for the treating or preventing an inflammation.

According to another embodiment, there is provided a use of a maple tree extract comprising at least one phytochemical according to the present invention for the inhibition of an α-glucosidase.

According to another embodiment, there is provided a use of a maple tree extract comprising at least one phytochemical according to the present invention for treating or preventing an inflammation.

According to another embodiment, there is provided a use of a maple tree extract comprising at least one phytochemical for the preparation of a medicament for treating or preventing a disease in a subject.

According to another embodiment, there is provided a use of a maple tree extract comprising at least one phytochemical according to the present invention for treating or preventing a disease in a subject.

The disease may be chosen from a cancer, a metabolic syndrome, a diabetes, a neurodegenerative disease, an oxidative stress related disease, an intestinal dysfunction, a heart disease, an inflammation and an inflammatory condition.

The intestinal dysfunction may be chosen from an inflammatory bowel disease, Crohn's disease, an ulcerative colitis, collagenous colitis, lymphocytic colitis, ischaemic colitis, diversion colitis, Behçet's disease, and indeterminate colitis.

The maple tree extract may be at least one of

a butanol extract from a maple syrup,

an ethyl acetate extract from a maple syrup,

a methanol extract from a maple syrup,

a methanol extract from a sugar maple leaf,

a methanol extract from a red maple leaf,

a methanol extract from a red maple stem,

a methanol extract from a sugar maple bark,

a methanol extract from a red maple bark,

a methanol extract from a red maple fruit,

a methanol extract from a red maple heartwood,

a methanol extract from a sugar maple heartwood,

an ethyl acetate extract from a sugar maple bark, and

a butanol extract from a sugar maple bark.

The at least one phytochemical may be from a butanol extract from a maple syrup which comprises a molecule chosen from
Lyoniresinol,
Secoisolariciresinol,
Dehydroconiferyl alcohol,
5′-methoxydehydroconiferyl alcohol,
(1,3-Propanediol, 1-(4-hydroxy-3-methoxyphenyl)-2-[4-[(1E)-3-hydroxy-1-propenyl]-2-methoxyphenoxy]-, (1R,2R)),
1-(4-hydroxy-3-methoxyphenyl)-2-[4-(3-hydroxypropyl)-2-methoxyphenoxy]-propane-1,3-diol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
Scopoletin,
Fraxetin,
(E)-3,3′-dimethoxy-4,4′-dihydroxystilbene,
2-hydroxy-3′,4′-dihydroxyacetophenone,
1-(2,3,4-trihydroxy-5-methylphenyl)-ethanone,
2,4,5-trihydroxyacetophenone,
Catechaldehyde,
Vanillin,
Syringaldehyde,
Gallic acid,
Trimethyl gallic acid methyl ester,
Syringic acid,
Syringenin,
(E)-coniferol,
C-veratroylglycol,
Catechol,
Quebecol,
Catechin, and
Epicatechin.

The at least one phytochemical may be from an ethyl acetate extract from a maple syrup which comprises a molecule chosen from:
Lyoniresinol,
Secoisolariciresinol,
1-(4-hydroxy-3-methoxyphenyl)-2-[4-(3-hydroxypropyl)-2 methoxyphenoxy]-propane-1,3-diol,
Scopoletin,
C-veratroylglycol,
5-(3″,4″-dimethoxyphenyl)-3-hydroxy-3-(4′-hydroxy-3′-methoxybenzyl)-4-hydroxymethyl-dihydrofuran-2-one,
(erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo,erythro)-1-[4-[(2-hydroxy-2-(4-hydroxy-3-methoxyphenyl)-1-(hydroxymethyl)ethoxy]-3-methoxyphenyl]-1,2,3-propanetriol,
(threo,threo)-1-[4-[(2-hydroxy-2-(4-hydroxy-3-methoxyphenyl)-1-(hydroxymethyl)ethoxy]-3-methoxyphenyl]-1,2,3-propanetriol,
threo-guaiacylglycerol-β-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Isolariciresinol,
Syringaresinol,
Icariside E4,
Sakuraresinol,
1,2-diguaiacyl-1,3-propanediol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
Dihydroconiferyl alcohol,
4-Acetylcatechol,
3′,4′,5′-Trihydroxyacetophenone,
3,4-Dihydroxy-2-methylbenzaldehyde,
Protocatechuic acid,
4-(dimethoxymethyl)-pyrocatechol,
Tyrosol,
Isofraxin, 4-hydroxycatechol, and
Phaseic acid.

The at least one phytochemical may be from a methanol extract from maple syrup which comprises a molecule chosen from:
Gallic acid,
(E)-3,3′-dimethoxy-4,4′-dihydroxy stilbene,
Syringic acid,
C-veratroylglycol,
1-(4-hydroxy-3-methoxyphenyl)-2-[4-(3-hydroxypropyl)-2-methoxyphenoxy]-propane-1,3-diol (guaiacylglycerol-(3-O-4′-dihydroconiferyl alcohol),
3-[(4-[(6-dexoy-α-L-mannopyranosyl)oxy]-3-methoxyphenyl)-5-(3,4-dimethoxyphenyl)dihydro-3-hydroxy-4-(hydroxymethyl)-2(3H)-furanone,
Lyoniresinol,
2-Hydroxy-3′,4′-dihydroxyacetophenone,
Syringenin,
Catechol,
Syringaldehyde,
Vanillin,
1,3-propanediol, 1-(4-hydroxy-3-Methoxyphenyl)-2-[4-[(1E)-3-hydroxy-1-propenyl]-2-methoxyphenoxy]-,(1R,2R),
2,3-dihydro-3-(hydroxymethyl)-2-(4-hydroxy-3-methoxyphenyl)-7-methoxy-5-benzofuranpropanol (dihydrodehydrodiconiferyl alcohol),
Ferulic acid,
Catechaldehyde,
Fraxetin,
(E)-coniferyl alcohol (coniferol),
Scopoletin,
1-(2,3,4-trihydroxy-5-methylphenyl)-ethanone,
p-coumaric acid,
Secoisolariciresinol,
Catechin,
Epicatechin,
3′,4′,5′-Trihydroxyacetophenone,
4-(dimethoxymethyl)-pyrocatechol,
4-acetylcatechol,
2,3-dihydroxy-1-(3,4-dihydroxyphenyl)-1-propanone,
Dihydroconiferyl alcohol,
Isofraxidin,
2,3-dihydroxy-1-(4-hydroxy-3,5-dimethoxyphenyl)-1-propanone,
Tyrosol,
3-hydroxy-1-(4-hydroxy-3,5-dimethoxyphenyl)propan-1-one,
Isolariciresinol,
5-(3″,4″-dimethoxyphenyl)-3-hydroxy-3-(4′-hydroxy-3′-methoxybenzyl)-4-hydroxymethyl-dihydrofuran-2-one,
Protocatechuic acid,
Threo-guaiacylglycerol-β-O-4′-dihydroconiferyl alcohol,
4-hydroxycatechol,
(erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
1,2-diguaiacyl-1,3-propanediol,
(threo,erythro) 1-[4-[(1R,2R)-2-hydroxy-2-(4-hydroxy-3-methoxyphenyl)-1-(hydroxymethyl)ethoxy]-3-methoxyphenyl]-1,2,3-propanetriol,
(threo,threo) 1-[4-[(1R,2R)-2-hydroxy-2-(4-hydroxy-3-methoxyphenyl)-1-(hydroxymethyl)ethoxy]-3-methoxyphenyl]-1,2,3-propanetriol,
Leptolepisol D,
Sakuraresinol,
(erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol,
Icariside E4,
Syringaresinol,
Acernikol,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
2-[4-[(2S,3R)-2,3-dihydro-3-(hydroxymethyl)-5-(3-hydroxy propyl)-7-methoxy-2-benzofuranyl]-2,6-dimethoxyphenoxy]-1-(4-hydroxy-3-methoxyphenyl)-1,3-propanediol, and
Buddenol E.

The at least one phytochemical may be from a methanol extract from a red maple bark which comprises a molecule chosen from:

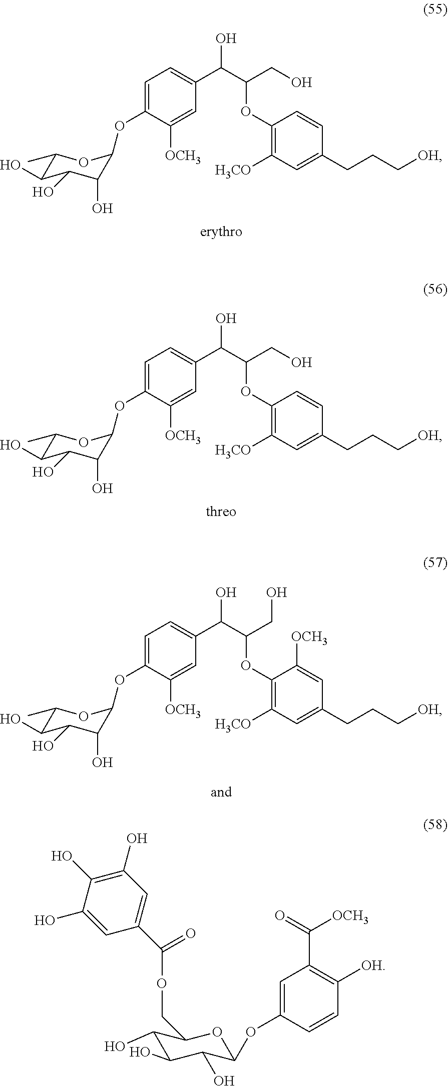 [SMILES: COc1cc(CCCO)ccc1OC(CO)C(O)c1ccc(OC2OC(C)C(O)C(O)C2O)c(OC)c1 ; COc1cc(CCCO)ccc1OC(CO)C(O)c1ccc(OC2OC(C)C(O)C(O)C2O)c(OC)c1 ; COc1cc(C(O)C(CO)Oc2c(OC)cc(CCCO)cc2OC)ccc1OC1OC(C)C(O)C(O)C1O ; COC(=O)c1cc(OC2OC(COC(=O)c3cc(O)c(O)c(O)c3)C(O)C(O)C2O)ccc1O]

The term “inflammatory condition is intended to mean a condition that results in abnormal inflammation, such as an allergic reaction, a myopathie, an immune disorder, cancer, atherosclerosis, and ischaemic heart disease.

The term “Acer tree” or a “maple tree” is intended to mean a maple tree of a species known to date, such as Acer nigrum, Acer lanum, Acer acuminatum, Acer albopurpurascens, Acer argutum, Acer barbinerve, Acer buergerianum, Acer caesium, Acer campbellii, Acer campestre, Acer capillipes, Acer cappadocicum, Acer carpinifolium, Acer caudatifolium, Acer caudatum, Acer cinnamomifolium, Acer circinatum, Acer cissifolium, Acer crassum, Acer crataegifolium, Acer davidii, Acer decandrum, Acer diabolicum, Acer distylum, Acer divergens, Acer erianthum, Acer erythranthum, Acer fabri, Acer garrettii, Acer glabrum, Acer grandidentatum, Acer griseum, Acer heldreichii, Acer henryi, Acer hyrcanum, Acer ibericum, Acer japonicum, Acer kungshanense, Acer kweilinense, Acer laevigatum, Acer laurinum, Acer lobelii, Acer lucidum, Acer macrophyllum, Acer mandshuricum, Acer maximowiczianum, Acer miaoshanicum, Acer micranthum, Acer miyabei, Acer mono, Acer mono x Acer truncatum, Acer monspessulanum, Acer negundo, Acer ningpoense, Acer nipponicum, Acer oblongum, Acer obtusifolium, Acer oliverianum, Acer opalus, Acer palmatum, Acer paxii, Acer pectinatum, Acer pensylvanicum, Acer pentaphyllum, Acer pentapomicum, Acer pictum, Acer pilosum, Acer platanoides, Acer poliophyllum, Acer pseudoplatanus, Acer pseudosieboldianum, Acer pubinerve, Acer pycnanthum, Acer rubrum, Acer rufinerve, Acer saccharinum, Acer saccharum, Acer sempervirens, Acer shirasawanum, Acer sieboldianum, Acer sinopurpurescens, Acer spicatum, Acer stachyophyllum, Acer sterculiaceum, Acer takesimense, Acer tataricum, Acer tegmentosum, Acer tenuifolium, Acer tetramerum, Acer trautvetteri, Acer triflorum, Acer truncatum, Acer tschonoskii, Acer turcomanicum, Acer ukurunduense, Acer velutinum, Acer wardii, Acer x peronai, Acer x pseudoheldreichii or any new species not yet known.

The term “sugar plant” is intended to mean any plant used in the production of sugar. Such plants include, without limitation, maple tree, birch tree, sugar cane, sugar beet, corn, rice, palm, and agave among others.

The term “metabolic syndrome” is intended to mean a combination of medical disorders that, when occurring together, increase the risk of developing cardiovascular disease and diabetes. The IDF consensus worldwide definition of the metabolic syndrome defines metabolic syndrome as: Central obesity (defined as waist circumference with ethnicity specific values) AND any two of the following: Raised triglycerides: >150 mg/dL (1.7 mmol/L), or specific treatment for this lipid abnormality. Reduced HDL cholesterol: <40 mg/dL (1.03 mmol/L) in males, <50 mg/dL (1.29 mmol/L) in females, or specific treatment for this lipid abnormality. Raised blood pressure: systolic BP>130 or diastolic BP>85 mm Hg, or treatment of previously diagnosed hypertension. Raised fasting plasma glucose: (FPG)>100 mg/dL (5.6 mmol/L), or previously diagnosed type 2 diabetes. If FPG>5.6 mmol/L or 100 mg/dL, OGTT Glucose tolerance test is strongly recommended but is not necessary to define presence of the Syndrome.

### BRIEF DESCRIPTION OF THE DRAWINGS

FIG. 1 illustrates HPLC-UV chromatogram of a butanol extract of Canadian maple syrup (1A) and twenty-three phenolic compounds isolated and identified therein (1B).

FIG. 2 illustrates the chemical structures of phenolic compounds 1-23 isolated and identified from a butanol extract of Canadian maple syrup.

FIG. 3 illustrates the chemical structure of phenolic compound of formula (54) named Quebecol.

FIG. 4 illustrates the chemical structure of phenolic compound (54) named Quebecol.

FIG. 5 illustrates the Structure of compounds 1-30 isolated and identified from Canadian maple syrup.

FIG. 6 illustrates (A) COSY (think lines) and HMBC (arrows) correlations for compound 1 and (B) NOE correlations for compound 24.

FIG. 7 Illustrates HPLC-UV chromatogram of 30 compounds isolated and identified from (A) an ethyl acetate extract of Canadian maple syrup (MS-EtOAc) combined in a single injection and (B) the whole MS-EtOAc extract

FIG. 8 Illustrates HPLC-UV chromatograms of maple syrup extracts. MS-BuOH (A), MS-MeOH (B) and MS-EtOAc (C) from grade C (1A-C) and grade D (1D-F), respectively. All extracts are injected at equivalent phenolic content. HPLC-UV Chromatogram of pure phenolic compounds isolated from maple syrup extracts (1G). Numbers correspond to the identities of compounds as shown in Tables 2-3.

FIG. 9 Illustrates the analysis of cell cycle distribution of cell lines treated with different maple syrup extracts. Distribution of cells in the G₀/G₁, S and G₂/M phases at 72 h: (A) HCT-116 cells, (B) Caco-2 cells, (C) HT-29 cells, (D) CCD-18Co cells. Data are expressed as mean values±SD (n=3). *p<0.05 indicate a significant difference compared to untreated cells.

FIG. 10 Illustrates the effect of maple syrup extracts on cyclins A and D1 expression in Caco-2 cells after 72 h of treatment. (*) Significant different densitometry p<0.05.

FIG. 11 illustrates the chemical structures of ginnalin-A (1), ginnalin-B (2) and ginnalin-C (3) isolated from Red maple twigs/stems and used for standardization of the maple plant part extracts. The molecular weights of compounds 70-72 are 468, 316, and 316 g/mol., respectively.

FIG. 12 illustrates a HPLC-UV chromatograms of maple plant part extracts showing the presence of ginnalins A-C in the Red maple (FIG. 12A) and Sugar maple (FIG. 12B) species. HPLC profiles are as follows: a=leaf; b=stem/twigs; c=bark; d=sapwood. Peak 1 (TR of 26 min)=ginnalin A; peak 2/3 co-eluting (TR of 15/16 min)=ginnalins-B and C, respectively. Chromatograms were monitored at a wavelength of 280 nm.

FIG. 13. Analysis of cell cycle distribution of cell lines treated with different extracts. Distribution of cells in the G₀/G₁, S and G₂/M phases at 72 h: (A) HCT-116 cells, (B) Caco-2 cells, (C) HT-29 cells, (D) CCD-18Co cells. Data are expressed as mean values±SD (n=3). *p<0.05 (two-tailed t test) indicate a significant difference compared to untreated cells.

FIG. 14 illustrates Yeast α-glucosidase inhibition of different maple syrup extracts standardized to the same phenolic content (3.75 mg/mL GAE). Different letters within the same doses indicate significant difference (p<0.05). (Bold: 187 μg; bold in parenthesis: 93.5 μg; bold underlined: 37.4 μg; italics: 18.7 μg; italics in parenthesis: 9.35 μg; italics underlined: 3.74 μg).

FIG. 15 illustrates rat α-glucosidase inhibitory activity of MS-EOAc and MS-BuOH maple syrup extracts standardized to the same phenolic content (3.75 mg/mL GAE).

FIG. 16 illustrates porcine α-amylase inhibitory activity of MS-EOAc and MS-BuOH maple syrup extracts standardized to the same phenolic content (3.75 mg/mL GAE).

### DETAILED DESCRIPTION OF THE PREFERRED EMBODIMENTS

In embodiments there are disclosed phenolic extracts and compounds from Canadian maple syrup (MS) and from maple trees (e.g. red, silver, or sugar maple). The compounds and extracts may be used for their cosmetological, cosmeceutical and nutraceutical properties, as functional food ingredients, as natural health product ingredients, for their therapeutic properties in the treatment or prevention of diseases such as, without limitations cancers, micro-organism infections (e.g. bacterial and/or fungal infections), a metabolic syndrome, a diabetes, a neurodegenerative disease, an oxidative stress related disease, an intestinal dysfunction, a heart disease, inflammation and an inflammatory condition.

In other embodiments there are disclosed Twenty-three phenolic compounds isolated from a butanol extract of Canadian maple syrup (MS) using chromatographic methods. The compounds are identified from their nuclear magnetic resonance and mass spectral data as seven lignans:

lyoniresinol (1), secoisolariciresinol (2), dehydroconiferyl alcohol (3), 5′-methoxy-dehydroconiferyl alcohol (4),erythro-guaiacylglycerol-β-O-4′-coniferyl alcohol (5),erythro-guaiacylglycerol-β-O-4′-dihydroconiferyl alcohol (6), and [3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone (7);

two coumarins: scopoletin (8) and fraxetin (9);

a stilbene: (E)-3,3′-dimethoxy-4,4′-dihydroxystilbene (10), and

thirteen phenolic derivatives: 2-hydroxy-3′,4′-dihydroxyacetophenone (11), 1-(2,3,4-trihydroxy-5-methylphenyl)-ethanone (12), 2,4,5-trihydroxyacetophenone (13), catechaldehyde (14), vanillin (15), syringaldehyde (16), gallic acid (17), trimethyl gallic acid methyl ester (18), syringic acid (19), syringenin (20), (E)-coniferol (21), C-veratroylglycol (22), and catechol (23).

The antioxidant activities of the MS extract, pure compounds, vitamin C (IC₅₀=58 μM), and the synthetic commercial antioxidant, butylatedhydroxytoluene (IC₅₀=2651 μM), are evaluated in the diphenylpicrylhydrazyl (DPPH) radical scavenging assay. Among the isolates, the phenolic derivatives and coumarins showed superior antioxidant activity (IC₅₀<100 μM) compared to the lignans and stilbene (IC₅₀>100 μM).

Also, this is the first report of phytochemicals 1, 2, 4-14, 18, 20 and 22 in MS.

General Experimental Procedures

¹H and ¹³C Nuclear Magnetic Resonance (NMR) spectra are obtained either on a Bruker™ 400 MHz or a Varian™ 500 MHz instrument using deuterated methanol (CD3OD) as solvent. Electrospray ionization mass spectral (ESIMS) data are acquired on a Q-Star Elite (Applied Biosystems MDS) mass spectrometer equipped with a Turbo lonspray source and are obtained by direct infusion of pure compounds. Analytical high performance liquid chromatography (HPLC) are performed on a Hitachi Elite LaChrom™ system consisting of a L2130 pump, L-2200 autosampler, and a L-2455 Diode Array Detector all operated by EZChrom™ Elite software. Semi-preparative scale HPLC are performed on a Beckman-Coulter HPLC system consisting of a Beckman System Gold™ 126 solvent module pump, 168 photodiode array (PDA)-UV/VIS detector, and 508 autosampler all operated by the 32 Karat 8.0 software. All solvents are either ACS or HPLC grade and are obtained from Wilkem Scientific (Pawcatuck, R.I.). Ascorbic acid (vitamin C), butylatedhydroxytoluene (BHT), and diphenylpicrylhydrazyl (DPPH) reagent are purchased from Sigma-Aldrich (St Louis, Mo.).

Maple Syrup (MS) Butanol Extract

Maple syrup (grade C, 20 L) is provided by the Federation of Maple Syrup Producers of Quebec (Canada). The syrup is kept frozen until extraction when it is subjected to liquid-liquid partitioning with ethyl acetate (10 L×3) followed by n-butanol (10 L×3) solvents, to yield ethyl acetate (4.7 g) and butanol (108 g) extracts, respectively, after solvent removal in vacuo.

Analytical HPLC

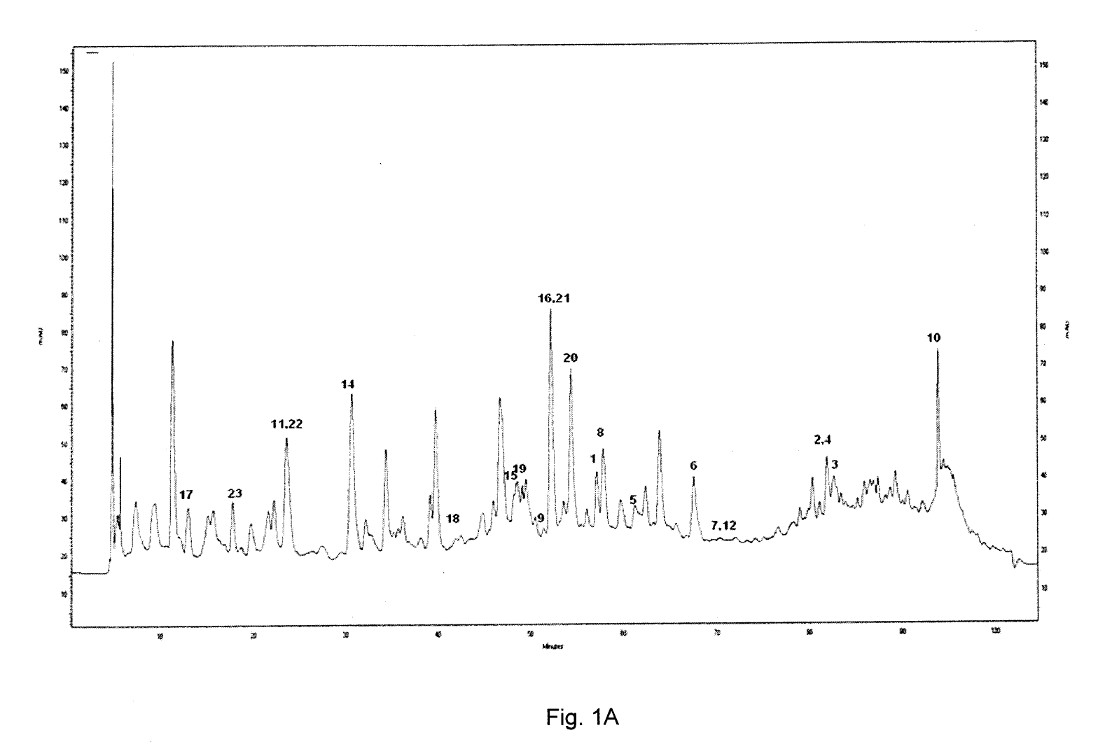

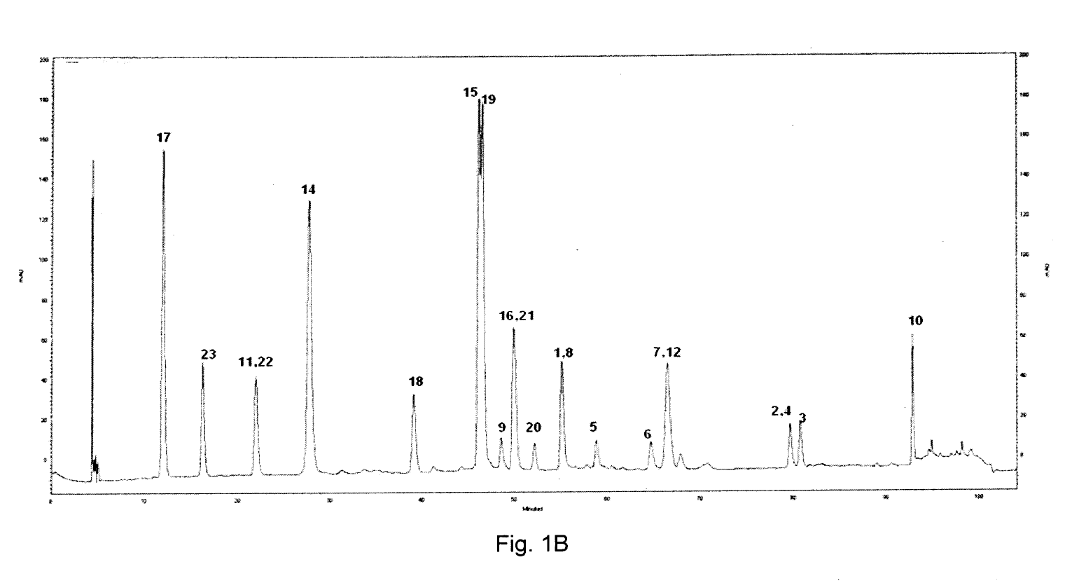

All analyses are conducted on a Luna C18 column (250×4.6 mm i.d., 5 μM; Phenomenex) with a flow rate at 0.75 mL/min and injection volume of 20 μL. A gradient solvent system consisting of solvent A (0.1% aqueous trifluoroacetic acid) and solvent B (methanol, MeOH) is used as follows: 0-10 min, 10% to 15% B; 10-20 min, 15% B; 20-40 min, 15% to 30% B; 40-55 min, 30% to 35% B; 55-65 min, 35% B; 65-85 min, 35% to 60% B; 85-90 min, 60% to 100% B, 90-93 min, 100% B; 93-94 min, 100% to 10% B; 94-104 min, 10% B. FIGS. 1A and 1B show the HPLC-UV profiles of the butanol extract and all of the isolated phenolics (combined into one solution/injection), respectively.

Isolation of Compounds from the MS Butanol Extract

The butanol extract (108 g) is extracted with methanol (100 mL×3) to afford methanol soluble (57 g; dark-brown powder) and methanol insoluble (51 g; off-white powder) fractions. Analytical HPLC analyses of the methanol soluble extract revealed a number of peaks characteristic of phenolic compounds at 220, 280 and 360 nm (see above for details of methodology; see FIG. 1A for chromatogram). Therefore, this fraction is selected for further purification by repeated chromatography on a Sephadex™ LH-20 column (4.5×64 cm), eluting with a gradient system of MeOH: H₂O (3:7 v/v to 7:3 v/v to 100:0 v/v), and then with acetone: H₂O (7:3 v/v). Based on analytical HPLC profiles, twelve combined fractions, Fr. 1-12, are obtained. Fr. 4 (1.5 g) is subjected to column chromatography on a Sephadex™ LH-20 column (4.5×64 cm) using a gradient solvent system of MeOH: H₂O (3:7 v/v to 7:3 v/v) to afford twelve sub-fractions, Fr. 4.1-4.12. These are individually subjected to a series of semi-prep HPLC separation using a Waters Sunfire Prep™ C₁₈ column (250×10 mm i.d., 5 μm; flow 2 mL/min) and eluting with a MeOH:H₂O gradient system to yield compounds 1 (4.6 mg), 3 (3.8 mg), 5 (4.0 mg), 6 (41.6 mg), 7 (6.6 mg), 11 (3.5 mg), 15 (0.3 mg), 16 (0.8 mg), 18 (0.2 mg), 20 (1.3 mg), 22 (1.5 mg) and 23 (3.0 mg). Similarly, Fr. 5 (0.47 g) is purified by semi-prep HPLC using a Waters XBridge Prep C₁₈ column (250×19 mm i.d., 5 μm; flow 3.5 mL/min) and a gradient solvent system of MeOH:H₂O to afford four subfractions Fr. 5.1-5.4. These subfractions are separately subjected to a combination of semi-prep HPLC and/or Sephadex™ LH-20 column chromatography with gradient solvents systems of MeOH:H₂O to afford compounds 2 (1.9 mg), 4 (1.9 mg), 8 (2.0 mg), 9 (2.3 mg), 14 (2.5 mg), 17 (2.4 mg), 19 (1.8 mg) and 21 (1.3 mg). Similarly, Fr. 6 (0.2 g) afforded compounds 12 (1.4 mg) and 13 (1.3 mg) and Fr. 11 yielded compound 10 (4.8 mg).

Isolation of Compounds from the MS Ethyl Acetate Extract

Maple syrup (grade C, 20 L) is provided by the Federation of Maple Syrup Producers of Quebec (Canada). The syrup (20 L) is kept in the freezer (−20° C.), until extraction when it is subjected to liquid-liquid partitioning with ethyl acetate (10 L×3) followed by n-butanol (10 L×3) solvents, to yield ethyl acetate (4.7 g) and butanol (108 g) extracts, respectively, after solvent removal in vacuo. The ethyl acetate extract (4.7 g) is subjected to a series of chromatographic isolation procedures using XAD-16, silica gel, Sephadex-LH 20, and C-18 column chromatography. Semi-purified fractions obtained from these columns are then further subjected to prep-HPLC to yield twenty pure compounds.

Identification of Compounds

All of the isolated compounds are identified by examination of their ¹H and/or ¹³C NMR and mass spectral data, and by comparison of these to published literature reports, when available (Tables 1). The NMR data for compounds 7, 12, and 13 are provided here.

TABLE 1
Compounds identified in a butanol extract of Canadian maple syrup (MS) and the literature references of
their previously reported nuclear magnetic resonance (NMR) data.

| | | | References |
|---|---|---|---|
| | Identification | Structure | with NMR data |
| 1 | lyoniresinol^{a,*} | 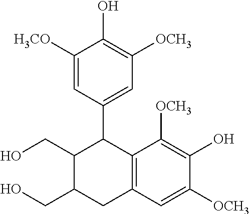 [SMILES: COc1cc(C2c3c(cc(OC)c(O)c3OC)CC(CO)C2CO)cc(OC)c1O] | Takemoto et al, Chem.Pharm. Bull. 2006, 54, 226-229 |
| 2 | secoisolariciresinol^{a,*} | 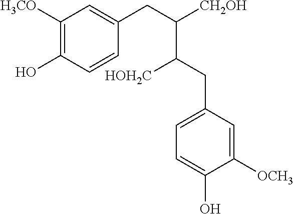 [SMILES: COc1cc(CC(CO)C(CO)Cc2ccc(O)c(OC)c2)ccc1O] | Baderschneider et al, J.Agric. Food Chem. 2001, 49, 2788-2798 |
| 3 | dehydroconiferyl alcohol^{a,b} | 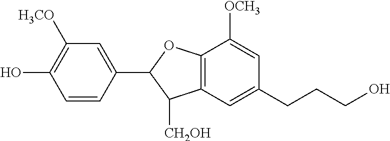 [SMILES: COc1cc(C2Oc3c(OC)cc(CCCO)cc3C2CO)ccc1O] | Junxiu et al, Org. Biomol. Chem., 2010, 8, 107-113 |
| 4 | 5′- methoxydehydroconiferyl alcohol^{a,*} | 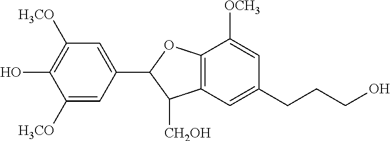 [SMILES: COc1cc(C2Oc3c(OC)cc(CCCO)cc3C2CO)cc(OC)c1O] | Chin et al, J. Agric. Food Chem. 2008, 56, 7759-7764 |
| 5 | guaiacylglycerol-β-O-4′- coniferyl alcohol^{a},^{*} (1,3-Propanediol, 1-(4- hydroxy-3-methoxyphenyl)- 2-[4-[(1E)-3-hydroxy-1- propenyl]-2- methoxyphenoxy]-, (1R, 2R)-) | 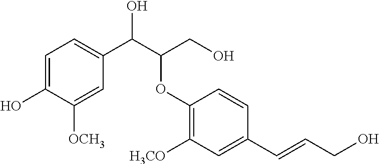 [SMILES: COc1cc(C(O)C(CO)Oc2ccc(/C=C/CO)cc2OC)ccc1O] | Han et al, J. Agric.Food Chem. 2008, 56, 6928-6935 |
| 6 | guaiacylglycerol-β-O-4′- dihydroconiferyl alcohol* 1-(4-hydroxy-3- methoxyphenyl)-2-[4-(3- hydroxypropyl)-2- methoxyphenoxy]-propane- 1,3-diol | 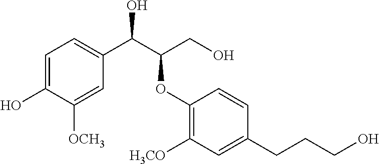 [SMILES: COc1cc([C@@H](O)[C@@H](CO)Oc2ccc(CCCO)cc2OC)ccc1O] | De Marino et al, Molecules 2008, 13, 1219-1229 |

Compounds identified in a butanol extract of Canadian maple syrup (MS) and the literature references of
their previously reported nuclear magnetic resonance (NMR) data.

| | | | References |
|---|---|---|---|
| | Identification | Structure | with NMR data |
| 1 | lyoniresinol^{a,*} | 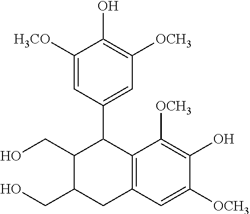 [SMILES: COc1cc(C2c3c(cc(OC)c(O)c3OC)CC(CO)C2CO)cc(OC)c1O] | Takemoto et al, Chem.Pharm. Bull. 2006, 54, 226-229 |
| 2 | secoisolariciresinol^{a,*} | 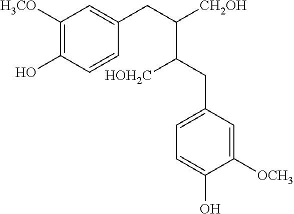 [SMILES: COc1cc(CC(CO)C(CO)Cc2ccc(O)c(OC)c2)ccc1O] | Baderschneider et al, J.Agric. Food Chem. 2001, 49, 2788-2798 |
| 3 | dehydroconiferyl alcohol^{a,b} | 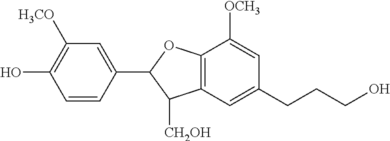 [SMILES: COc1cc(C2Oc3c(OC)cc(CCCO)cc3C2CO)ccc1O] | Junxiu et al, Orgi Biomol. Chem., 2010, 8, 107-113 |
| 4 | 5′- methoxydehydroconiferyl alcohol^{a,*} | 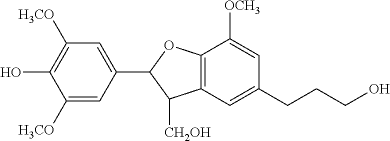 [SMILES: COc1cc(C2Oc3c(OC)cc(CCCO)cc3C2CO)cc(OC)c1O] | Chin et al, J. Agric. Food Chem. 2008, 56, 7759-7764 |
| 5 | guaiacylglycerol-β-O-4′- coniferyl alcohol^{a},^{*} (1,3-Propanediol, 1-(4- hydroxy-3-methoxyphenyl)- 2-[4-[(1E)-3-hydroxy-1- propenyl]-2- methoxyphenoxy]-, (1R, 2R)-) | 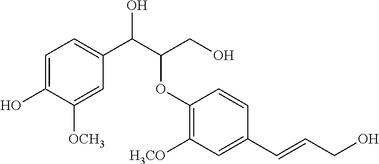 [SMILES: COc1cc(C(O)C(CO)Oc2ccc(/C=C/CO)cc2OC)ccc1O] | Han et al, J. Agric.Food Chem. 2008, 56, 6928-6935 |
| 6 | guaiacylglycerol-β-O-4′- dihydroconiferyl alcohol* 1-(4-hydroxy-3- methoxyphenyl)-2-[4-(3- hydroxypropyl)-2- methoxyphenoxy]-propane- 1,3-diol | 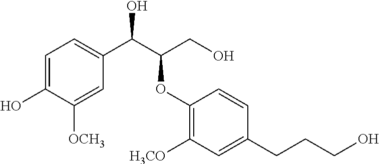 [SMILES: COc1cc([C@@H](O)[C@@H](CO)Oc2ccc(CCCO)cc2OC)ccc1O] | De Marino et al, Molecules 2008, 13, 1219-1229 |
| 7 | [3-[4-[(6-deoxy-α-L- mannopyranosyl)oxy]- 3-methoxyphenyl]methyl]- 5-(3,4-dimethoxyphenyl) dihydro-3-hydroxy-4- (hydroxymethyl)-2(3H)- furanone^{b,*} | 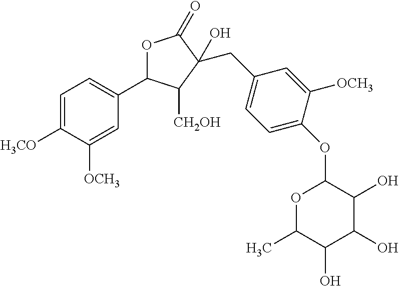 [SMILES: COc1ccc(C2OC(=O)C(O)(Cc3ccc(OC4OC(C)C(O)C(O)C4O)c(OC)c3)C2CO)cc1OC] | NA |
| 8 | scopoletin^{a,b*} | 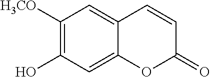 [SMILES: COc1cc2ccc(=O)oc2cc1O] | Yoshikawa et al, Bios. Biotechnol. and Biochem. 2003, 67, 2408-2415 |
| 9 | fraxetin^{a,*} | 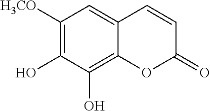 [SMILES: COc1cc2ccc(=O)oc2c(O)c1O] | Liu et al, J. Chrom. A, 2005, 1072, 195-199 |
| 10 | (E)-3,3′-dimethoxy-4,4′- dihydroxystilbene^{a,*} | 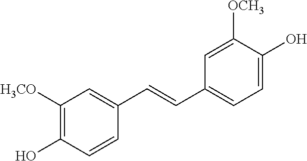 [SMILES: COc1cc(/C=C/c2ccc(O)c(OC)c2)ccc1O] | Hajdu et al, J. Nat.Prod. 1998, 61, 1298-1299 |
| 11 | 2-hydroxy-3′,4′- dihydroxyacetophenone^{a,*} | 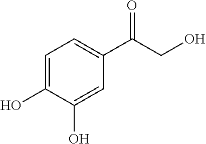 [SMILES: O=C(CO)c1ccc(O)c(O)c1] | Tsuda et al, J. Agric.Food Chem. 1994, 42, 2671-2674 |
| 12 | 1-(2,3,4-trihydroxy-5- methylphenyl)-ethanone^{b,*} | 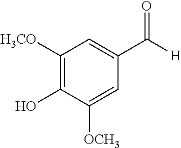 [SMILES: COc1cc(C=O)cc(OC)c1O] | NA |
| 13 | 2,4,5- trihydroxyacetophenone^{b,*} | 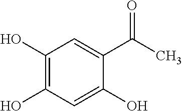 | NA |
| 14 | catechaldehyde^{a,*} | 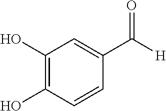 | Prachayasittikul et al, Molecules 2008, 13, 904-921 |
| 15 | vanillin^{a} | 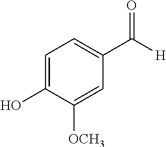 [SMILES: COc1cc(C=O)ccc1O] | Bonini et al, Photochem. Photobiol.Sci. 2002, 1, 570-573 |
| 16 | syringaldehyde^{a} | 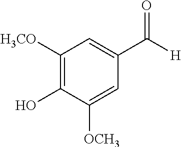 [SMILES: COc1cc(C=O)cc(OC)c1O] | Bonini et al, Photochem. Photobiol. Sci. 2002, 1, 570-573 |
| 17 | gallic acid^{a} | 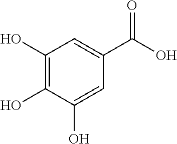 [SMILES: O=C(O)c1cc(O)c(O)c(O)c1] | Le gall et al, J. Agric. Food Chem. 2004, 52, 692-700 |
| 18 | trimethyl gallic acid methyl ester^{a,*} | 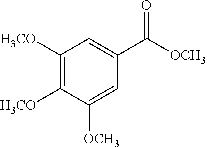 [SMILES: COC(=O)c1cc(OC)c(OC)c(OC)c1] | Avila-Zarrage et al, Syn. Commun. 2001, 31, 2177-2183 |
| 19 | syringic acid^{a} | 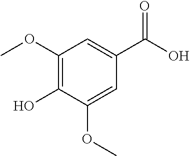 | Bonini et al, Photochem. Photobiol. Sci. 2002, 1, 570-573 |
| 20 | syringenin^{a,*} | 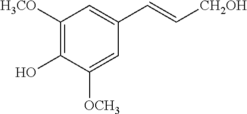 [SMILES: COc1cc(/C=C/CO)cc(OC)c1O] | Bonini et al, Photochem. Photobiol. Sci. 2002, 1, 570-573 |
| 21 | (E)-coniferol^{a} | 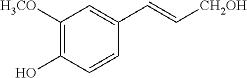 [SMILES: COc1cc(/C=C/CO)ccc1O] | Yao et al, Chem. Pharm. Bull. 2006, 54, 1053-1057 |
| 22 | C-veratroylglycol^{a,*} | 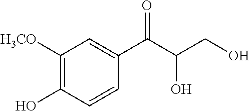 [SMILES: COc1cc(C(=O)C(O)CO)ccc1O] | Baderschneider et al, J. Agric. Food Chem. 2001, 49, 2788-2798 |
| 23 | catechol^{a} | 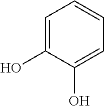 | Loo et al, Food Chem. 2007, 107, 1151-1160 |
| 54 | Quebecol | 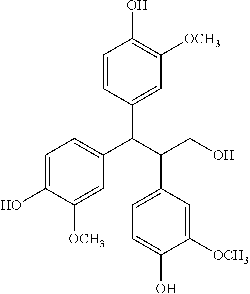 [SMILES: COc1cc(C(CO)C(c2ccc(O)c(OC)c2)c2ccc(O)c(OC)c2)ccc1O] | |
| 59 | Catechin | | |
| 60 | Epicatechin | | |

NA = none-available.
^{a}Identified by examination and comparison of NMR and mass spectral data to literature reports
^{b}Compounds described in Japenese patents (27, 28) but no NMR data provided
^{*}First peer-review report from maple syrup

(+)-Lyoniresinol (1).

Yellowish amorphous powder; (+) ESIMS, m/z 443.1719 [M+Na]⁺, calcd. for molecular formula C₂₂H₂₈O₈; (400 MHz) ¹H and ¹³C NMR data are consistent with literature.

Secoisolariciresinol (2).

Yellowish amorphous powder; (+) ESIMS m/z 385.1447 [M+Na]⁺, calcd. for molecular formula C₂₀H₂₆O₆; (500 MHz) ¹H and ¹³C NMR data are consistent with literature.

Dehydroconiferyl Alcohol (3).

Yellowish amorphous powder; (+) ESIMS m/z 383.1208 [M+Na]⁺, calcd. for molecular formula C₂₀H₂₄O₆; (400 MHz) ¹H and ¹³C NMR data are consistent with literature.

5-methoxydehydroconiferyl Alcohol (4).

Yellowish amorphous powder; (+) ESIMS m/z 413.1464 [M+Na]⁺, calcd. for molecular formula C₂₁H₂₆O₇; (500 MHz) ¹H and ¹³C NMR data are consistent with literature.

Erythro-guaiacylglycerol-β-O-4′-coniferyl alcohol (5).

Yellowish amorphous powder; (+) ESIMS m/z 399.1156 [M+Na]⁺, calcd. for molecular formula C₂₀H₂₄O₇; (400 MHz) ¹H and ¹³C NMR data are consistent with literature.

Erythro-guaiacylglycerol-beta-O-4′-dihydroconiferyl alcohol (6).

Yellowish amorphous powder; (+) ESIMS m/z 401.1602 [M+Na]⁺, calcd. for molecular formula O₂₀H₂₆O₇; (400 MHz) ¹H and ¹³C NMR data are consistent with literature.

[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone (7). Yellowish amorphous powder; (+) ESIMS m/z 573.1913 [M+Na]⁺, calcd. for molecular formula C₂₇¹¹₃₄O₁₂.

(400 MHz) ¹H NMR: δ 7.05 (1H, d, J=8.4 Hz, H-5), 6.97 (1H, s, H-2), 6.87 (1H, d, J=8.4 Hz, H-5′), 6.85 (1H, d, J=8.0 Hz, H-6), 6.62 (1H, d, J=8.0 Hz, H-6′), 6.37 (1H, s, H-2′), 5.31 (1H, s, H-1″), 5.10 (1H, d, J=9.2 Hz, H-7′), 4.07 (1H, s, H-2″), 3.95 (1H, m, 9′a), 3.80 (3H, s, 3-OCH₃), 3.79 (3H, s, 3′-OCH₃), 3.63 (3H, s, 4′-OCH₃), 3.55 (1H, m, 9′b), 3.5-3.90 (3H, m, H-3″, 4″, 5″), 3.35 (1H, d, J=13.2 Hz, H-7a), 3.06 (1H, d, J=13.2 Hz, H-7b), 1.25 (3H, d, J=6.4 Hz, H-6″). (100 MHz) ¹³C NMR: δ 179.64 (C-9), 152.11 (C-3), 151.04 (C-3′), 150.74 (C-4′), 146.15 (C-4), 132.66 (C-1), 132.45 (C-1′), 124.54 (C-6), 120.92 (C-6′), 120.11 (C-5), 116.36 (C-2), 112.60 (C-5′), 110.39 (C-2′), 101.82 (C-1″), 82.89 (C-7′), 79.47 (C-8), 73.94 (C-4″), 72.33 (C-3″), 72.25 (C-2″), 71.02 (C-5″), 58.69 (C-9′), 56.75, 56.50 (C-3, 3′, 4′-OCH₃), 51.79 (C-8′), 42.75 (C-7), 18.18 (C-6″).

Scopoletin (8).

Yellowish amorphous powder; (+) ESIMS m/z 193.0787 [M+H]⁺, calcd. for molecular formula C₁₀H₈O₄; (500 MHz) ¹H NMR data are consistent with literature.

Fraxetin (9).

Yellowish amorphous powder; (+) ESIMS m/z 209.0639 [M+H]⁺, calcd. for molecular formula C₁₀H₈O₅; (400 MHz) ¹H NMR data are consistent with literature.

(E)-3,3′-dimethoxy-4,4′-dihydroxystilbene (10).

Yellowish amorphous powder; (+) ESIMS m/z 294.9650 [M+Na]⁺, calcd. for molecular formula O₁₆H₁₆O₄; (400 MHz) ¹H and ¹³C NMR data are consistent with the literature.

2-hydroxy-3′,4′-dihydroxyacetophenone (11).

Brown amorphous powder; (+) ESIMS m/z 191.0227 [M+Na]⁺, calcd. for molecular formula C₈H₈O₄; (500 MHz) ¹H NMR data are consistent with the literature.

1-(2,3,4-trihydroxy-5-methylphenyl)-ethanone (12).

Brown amorphous powder; (−) ESIMS m/z 181.0691 [M−H]⁻, calcd. for molecular formula C₉H₁₀O₄; (500 MHz) ¹H NMR: δ 7.08 (1H, s, H-7), 2.51 (3H, s, CH₃C0), 2.15 (3H, s, CH₃).

2,4,5-trihydroxyacetophenone (13).

Brown amorphous powder; (−) ESIMS m/z 167.0601 [M−H]⁻; calcd. for molecular formula C₈H₈O₄; (500 MHz) ¹H NMR: δ 7.16 (1H, s, H-7), 6.28 (1H, s, H-5), 2.48 (31-1, CH₃).

Catechaldehyde (14).

Brown amorphous powder; (−) ESIMS m/z 137.0341 [M−H]⁻, calcd. for molecular formula C₇H₆O₃; (400 MHz) ¹H NMR data are consistent with literature.

Vanillin (15).

White amorphous powder; (−) ESIMS m/z 151.0667 [M−H]⁻, calcd. for molecular formula C₈H₃O₂; (500 MHz) ¹H NMR data are consistent with the literature.

Syringaldehyde (16).

White amorphous powder; (−) ESIMS m/z 181.0768 [M−H]⁻, calcd. for molecular formula C₉H₁₀O₄; (500 MHz) ¹H NMR data are consistent with literature.

Gallic Acid (17).

Brown amorphous powder; (−) ESIMS m/z 169.1226 [M−H]⁻, calcd. for molecular formula C₇H₆O₅; (400 MHz) ¹H NMR data are consistent with the literature.

Trimethylgallic Acid Methyl Ester (18).

Brown amorphous powder; (+) ESIMS m/z 249.0735 [M+Na]⁺, calcd. for molecular formula C₁₁H₁₄O₅; (400 MHz) ¹H NMR data are consistent with the literature.

Syringic Acid (19).

White amorphous powder; (−) ESIMS m/z 197.0256 [M−H]⁻, calcd. for molecular formula C₉H₁₀O₅; (400 MHz) ¹H NMR data are consistent with literature.

Syringenin (20).

Brown amorphous powder; (+) ESIMS m/z 233.0630 [M+Na]⁺, calcd. for molecular formula C₁₁H₁₄O₄; (500 MHz) ¹H NMR data are consistent with literature.

(E)-coniferol (21).

Brown amorphous powder; (−) ESIMS m/z 179.0833 [M−H]⁻, calcd. for molecular formula C₀H₁₂O₃; (400 MHz) ¹H NMR data are consistent with literature.

C-veratroylglycol (22).

Brown amorphous powder; (+) ESIMS m/z 235.0582 [M+Na]⁺, calcd. for molecular formula C₁₀H₁₂O₅; (400 MHz) ¹H and ¹³C NMR data are consistent with literature.

Catechol (23).

Brown amorphous powder; (−) ESIMS m/z 109.0448 [M−H]⁻, calcd. for molecular formula r C₆H₆O₂; (400 MHz) ¹H and ¹³C NMR data are consistent with the literature.

Isolation and Identification of Compounds in MS Butanol Extract

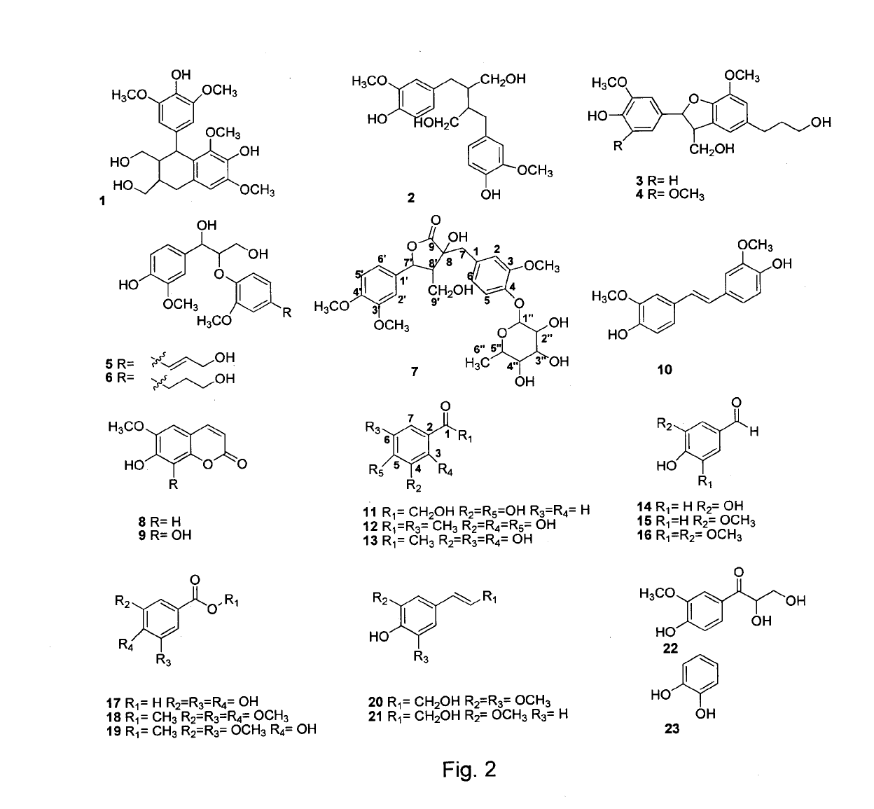

FIG. 1A shows the HPLC-UV profile of the MS butanol extract which revealed several peaks at 280 and 360 nm which are characteristic of phenolic compounds. The extract is subjected to a series of chromatographic isolation procedures to yield twenty-three (1-23) phenolics. FIG. 1B shows the HPLC-UV profile of the purified isolates all combined into a single injection. All of the compounds are identified based on their ¹H and ¹³C NMR and mass spectral data and by correspondence to published literature data where available (Table 1). FIG. 2 shows the structures of the compounds and they are grouped into their individual phenolic sub-classes for ease of discussion as follows.

Lignans.

Seven lignans are isolated from the MS butanol extract and identified as lyoniresinol (1), secoisolariciresinol (2), dehydroconiferyl alcohol (also known as dihydrodehydrodiconiferyl alcohol) (3), 5′-methoxydehydroconiferyl alcohol (4),erythro-guaiacylglycerol-β-O-4′-coniferyl alcohol (5),erythro-guaiacylglycerol-p-O-4′-dihydroconiferyl alcohol (6), and [3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone (7)

With the exception of dehydroconiferyl alcohol (3), which has been previously reported as a lignin-derived flavor compound in MS, this is the first reported occurrence of all of the other lignans in MS. However, it should be noted that compound 7 has been previously described as a constituent present in sap of Acer species in Japanese patent applications (Arihara, S. et al., in the name of Jpn. Kokai Tokkyo Koho, JP 2006008523 A, Publication number 2006-008523; Yoshikawa, K. et al., in the name of Jpn. Kokai Tokkyo Koho, JP 2009067718 A, Publication number 2009-067718) but there are no peer-reviewed reports to support its occurrence in MS. Also, apart from dehydroconiferyl alcohol (3), previously found in MS, and lyoniresinol (1), previously reported from leaves of A. truncatum (Dong, L. P.; Ni et al., Molecules. 2006, 11, 1009-1014), this is the first report of all of the other lignans in the Acer genus.

Lignan-rich foods, such as flaxseed which contains secoisolariciresinol (2), have attracted significant research attention for their biological effects. Thus the presence of these compounds in MS is interesting from a human health perspective. However, determination of the levels of these lignans (as well as the other bioactive phenolic sub-classes described below) in different grades of MS consumed by humans, and whether these compounds achieve physiologically relevant levels after MS consumption, would be required to evaluate their impact on human health.

Coumarins.

Two coumarins, not previously reported from MS, are isolated from the butanol extract and identified as scopoletin (8) and fraxetin (9). Similar to compound 7, scopoletin (8) has also been described in the aforementioned Japanese patents, but this is the first peer-reviewed report of its occurrence in MS. Also, while scopoletin (8) has been previously isolated from the bark of A. nikoense (Inoue, T et al., Yakugaku Zasshi, 1978, 98, 41-46), this is the first report of fraxetin (9) in the Acer genus.

Stilbene.

A stilbene is isolated from the butanol extract of MS and identified as (E)-3,3′-dimethoxy-4,4′-dihydroxystilbene (10). While stilbene glycosides have been previously reported from the leaves of A. mono (Yang, H et al., J. Nat. Prod. 2005, 68, 101-103), this is the first reported occurrence of a stilbenoid in MS. Foods containing stilbenes have attracted immense public attention for their potential human health benefits due in large part to emerging research on resveratrol, a stilbene present in red wine, grapes, and berries.

Phenolic Derivatives.

Thirteen phenolic derivatives are found in MS including 2-hydroxy-3′,4′-dihydroxyacetophenone (11), 1-(2,3,4-trihydroxy-5-methylphenyl)-ethanone (12), 2,4,5-trihydroxyacetophenone (13), catechaldehyde (14), vanillin (15), syringaldehyde (16), gallic acid (17), trimethyl gallic acid methyl ester (18), syringic acid (19) syringenin (20), (E)-coniferol (21), C-veratroylglycol (22), and catechol (23). While several of these compounds have been previously found in MS, this is the first report of catechaldehyde (14), trimethyl gallic acid methyl ester (18), syringenin (20) and C-veratroylglycol (22) in MS.

Other Unidentified Compounds.

It is noteworthy that a number of peaks/compounds in MS still remain unidentified (FIG. 1A). Despite starting our initial extraction scheme with 20 L of MS, several compounds are unobtainable either due to rapid degradation/decomposition on our columns and/or low yields.

In another embodiment, there are disclosed thirty phenolics obtained from an ethyl acetate extract of maple syrup (MS-EtOAc).

TABLE 2
Total Compounds isolated from an Ethyl Acetate Extract of Canadian Maple Syrup (MS-EtOAc)

| compd | identification | references of NMR data |
|---|---|---|
| 1 | Lyoniresinol | |
| 2 | Secoisolariciresinol | |
| 6 | 1-(4-hydroxy-3-methoxyphenyl)-2-[4-(3-hydroxypropyl)-2-methoxyphenoxy]-propane- | |
| | 1,3-diol (guaiacylglycerol-β-O-4′-dihydroconiferyl alcohol) | |
| 8 | Scopoletin | |
| 22 | C-veratroylglycol | |
| 24 | 5-(3″,4″-dimethoxyphenyl)-3-hydroxy-3-(4′-hydroxy-3′-methoxybenzyl)-4- | — |
| | hydroxymethyl-dihydrofuran-2-one* | |
| 25 | (erythro, erythro)-1-[4-[2-hydroxy-2-(4-hydroxy-3-methoxyphenyl)-1- | — |
| | (hydroxymethyl)ethoxy]-3,5-dimethoxyphenyl]-1,2,3-propanetriol* | |
| 26 | (erythro, threo)-1-[4-[2-hydroxy-2-(4-hydroxy-3-methoxyphenyl)-1- | — |
| | (hydroxymethyl)ethoxy]-3,5-dimethoxyphenyl]-1,2,3-propanetriol* | |
| 27 | (threo, erythro)-1-[4-[(2-hydroxy-2-(4-hydroxy-3-methoxyphenyl)-1- | Della-Grace et al, Phytochemistry |
| | (hydroxymethyl)ethoxy]-3-methoxyphenyl]-1,2,3-propanetriol^{a} | 1998, 49, 1299-1304. |
| 28 | (threo, threo)-1-[4-[(2-hydroxy-2-(4-hydroxy-3-methoxyphenyl)-1- | Della-Grace et al, Phytochemistry |
| | (hydroxymethyl)ethoxy]-3-methoxyphenyl]-1,2,3-propanetriol^{a} | 1998, 49, 1299-1304. |
| 29 | threo-guaiacylglycerol-β-O-4′-dihydroconiferyl alcohol | J. Asian Nat. Prod. Res. 2007, |
| | | 9, 583-591 |
| 30 | erythro-1-(4-hydroxy-3-methoxyphenyl)-2-[4-(3-hydroxypropyl)-2,6- | Jutiviboonsuk et al, Phytochemistry |
| | dimethoxyphenoxy]-1,3-propanediol^{a} | 2005, 66, 2745-2751 |
| 31 | 2-[4-[2,3-dihydro-3-(hydroxymethyl)-5-(3-hydroxypropyl)-7-methoxy-2-benzofuranyl]- | — |
| | 2,6-dimethoxyphenoxy]-1-(4-hydroxy-3-methoxyphenyl)-1,3-propanediol^{b} | |
| 32 | acernikol | Morikawa et al, Chem. Pharm. Bull. |
| | | 2003, 51, 62-67 |
| 33 | leptolepisol D^{a,b} | — |
| 34 | buddlenol E^{a} | Houghton et al, Phytochemistry |
| | | 1985, 24, 819-826 |
| 35 | (1S,2R)-2-[2,6-dimethoxy-4-[(1S,3aR,4S,6aR)-tetrahydro-4-(4-hydroxy-3,5- | Fiorentino et al, Biochem. Syst. |
| | dimethoxyphenyl)-1H,3H-furo[3,4-c]furan-1-yl]phenoxy]-1-(4-hydroxy-3- | Eco. 2007, 35, 392-396 |
| | methoxyphenyl)-1,3-propanediol^{a} | |
| 36 | syringaresinol | Cai et al, Arch. Pharm. Res. 2004, |
| | | 27, 738-741 |
| 37 | isolariciresinol^{a} | Erdemoglu et al, J. Mol. Struct. |
| | | 2003, 655, 459-466 |
| 38 | icariside E4^{a} | Nakanishi et al, Phytochemistry |
| | | 2004, 65, 207-213 |
| 39 | sakuraresinol^{a} | Yoshinari et al, Phytochemistry |
| | | 1990, 29, 1675-1678 |
| 40 | 1,2-diguaiacyl-1,3-propanediol^{a} | Yoshiwara et al, J. Nat. Prod. 1998, |
| | | 61, 1137-1139 |
| 41 | 2,3-dihydroxy-1-(3,4-dihydroxyphenyl)-1-propanone* | — |
| 42 | 2,3-dihydroxy-1-(4-hydroxy-3,5-dimethoxyphenyl)-1-propanone^{a} | Lee et al, J. Nat. Prod. 2002, |
| | | 65, 1497-1500 |
| 43 | 3-Hydroxy-1-(4-hydroxy-3,5-dimethoxyphenyl)propan-1-one^{a} | Jones et al, Fitoterapia |
| | | 2000, 71, 580-583 |
| 44 | dihydroconiferyl alcohol^{b} | — |
| 45 | 4-acetylcatechol^{a} | Xiao et al, Bioorg. Med. Chem. |
| | | 2007, V15, 3703-3710 |
| 46 | 3′,4′,5′-trihydroxyacetophenone^{a,b} | — |
| 47 | 3,4-dihydroxy-2-methylbenzaldehyde^{a,b} | — |
| 48 | protocatechuic acid | Zhang et al, Phytochemistry |
| | | 1998, 48, 665-668 |
| 49 | 4-(dimethoxymethyl)-pyrocatechol^{a,b} | — |
| 50 | tyrosol | Takaya et al, J. Agric. Food Chem. |
| | | 2007, 55, 75-79 |
| 51 | isofraxidin^{a} | Okuyama et al, Chem. Pharm. Bull. |
| | | 2001, 49, 154-160 |
| 52 | 4-hydroxycatechol^{a} | Hiramoto et al, Mutat. Res. Gene. |
| | | Toxicol. Envir. Mutagen. |
| | | 1998, 419, 43-51 |
| 53 | phaseic acid^{a} | Hirai et al, Bios. Biotechnol. and |
| | | Biochem 2003, 67, 2408-2415 |

^{a}First report from maple syrup
^{b}NMR data provided for the first time herein
*New compounds

General Experimental Procedures.

All 1D proton and carbon-13 Nuclear Magnetic Resonance (¹H and ¹³C-NMR) and 2D NMR experiments, ¹H-¹H correlation spectroscopy (COSY), HSQC (Heteronuclear Single Quantum Coherence), HMBC (Heteronuclear Multiple Bond Coherence), and NOE (Nuclear Overhauser Effect), are acquired either on a Bruker 400 MHz or on a Varian 500 MHz instrument. Unless otherwise stated, deuterated methanol (CD₃OD) is used as solvent. High resolution electrospray ionization mass spectral (HRESIMS) data are acquired on a Q-Star Elite (Applied Biosystems MDS) mass spectrometer equipped with a Turbo lonspray source and is obtained by direct infusion of the pure compounds. Analytical and semi-preparative high performance liquid chromatography (HPLC) are performed on a Hitachi Elite LaChrom system consisting of a L2130 pump, L-2200 autosampler, and a L-2455 Diode Array Detector all operated by EZChrom Elite software. Medium-pressure liquid chromatography (MPLC) is carried out on prepacked C18 columns connected to a DLC-10/11 isocratic liquid chromatography pump (D-Star Instruments, Manassas, Va.) with a fixed-wavelength detector. Optical rotation is performed on an Auto Pol III Automatic Polarimeter (Rudolph Research, Flanders, N.J., USA) with samples dissolved in methanol at 22° C. using a 1 dm pathway cell.

Chemicals and Reagents.

All solvents are of ACS or HPLC grade and are obtained from Sigma-Aldrich through Wilkem Scientific (Pawcatuck, R.I.). Sephadex LH-20, ascorbic acid, butylated hydroxytoluene (BHT), and diphenylpicrylhydrazyl (DPPH) reagent are purchased from Sigma-Aldrich (St. Louis, Mo.).

Extraction and Isolation of Maple Syrup Ethyl Acetate (MS-EtOAc) Compounds.

Maple syrup (grade C, 20 L) is provided by the Federation of Maple Syrup Producers of Quebec (Canada). The maple syrup is shipped and kept frozen upon delivery. The maple syrup is subjected to liquid-liquid partitioning with ethyl acetate (10 L×3) to yield a dried ethyl acetate extract (MS-EtOAc; 4.7 g) after solvent removal in vacuo. The MS-EtOAc (4.5 g) is initially purified on a Sephadex LH-20 column (4×65 cm) with a gradient system of MeOH/H₂O (3:7 to 1:0, v/v) to afford seven fractions, A1-A7. Fraction A1 (2.08 g) is then chromatographed on a C18 MPLC column (4×37 cm) eluting with a gradient system of MeOH/H₂O (3:7 to 1:0, v/v) to afford sixteen subfractions, B1-B16. These sub-fractions are individually subjected to a series of semi-preparative HPLC separations using a Phenomenex Luna C18 column (250×10 mm i.d., 5 μm, flow=2 mL/min) with different isocratic elution systems of MeOH/H₂O to afford compounds 25 (0.9 mg), 26 (2.5 mg), 27 (0.8 mg), 28 (0.5 mg), 29 (17.5 mg), 730 (0.7 mg), 31 (1.1 mg), 32 (3.9 mg), 33 (1.1 mg), 34 (2.1 mg), 35 (2.8 mg), 36 (3.2 mg), 38 (2.4 mg), 39 (5.2 mg), 40 (0.8 mg), and 53 (0.5 mg). Similarly, fraction A3 (0.71 g) is purified by semi-preparative HPLC using a Waters XBridge Prep C18 column (250×19 mm i.d., 5 μm; flow=3.5 mL/min) and a gradient solvent system of MeOH/H₂O to afford four subfractions C1-C4. These subfractions are separately subjected to semi-preparative HPLC with isocratic solvents systems of MeOH/H₂O to afford compounds 24 (2.2 mg), 37 (4.5 mg), 42 (4.5 mg), 43 (2.2 mg), 44 (4.2 mg), 50 (3.7 mg), and 51 (1.1 mg). Similarly, fraction A4 (0.097 g) is purified by semi-preparative HPLC to afford compounds 41 (1.4 mg), 45 (2.6 mg), 46 (8.0 mg), 47 (0.4 mg), and 49 (3.2 mg) and subfraction A5 (0.022 g) yielded compounds 48 (3.6 mg) and 52 (1.1 mg).

TABLE 3
¹H-NMR [δ, (multiplicity, J_{HH} in Hz)] Spectroscopic Data for
Compounds 24-26 and 41

| No. | 24 | 25 | 26 | 41^{a} |
|---|---|---|---|---|
| 2 | 6.88 (s) | 6.99 (s) | 6.91 (s) | 7.45 (s) |
| 5 | 6.74 (brs) | 6.74 (d, | 6.64 (d, 8.5) | 7.47 (d, 8.0) |
| | | overlapped) | | |
| 6 | 6.74 (brs) | 6.77 (d, | 6.77 (d, 8.5) | 6.85 (d, 8.0) |
| | | overlapped) | | |
| 7a | 3.01 (dd, 13.0, 1.5) | 4.91 (d, 4.5) | 4.89 (d, 7.0) | — |
| 7b | 3.38 (d, 12.5) | — | — | |
| 8 | — | 4.21 (m) | 3.92 (m) | 5.09 (brs) |
| 9a | — | 3.90 (m) | 3.30 (m) | 3.88 (d, 8.8) |
| 9b | — | 3.50 (m) | 3.66 (dd, | 3.73 (m) |
| | | | 12.0, 4.0) | |
| 2′ | 6.23 (brs) | 6.75 (s) | 6.66 (s) | — |
| 5′ | 6.82 (dd, 8.0, 1.5) | — | — | — |
| 6′ | 6.68 (d, 8.0) | 6.75 (s) | 6.66 (s) | — |
| 7′ | 5.08 (dd, 9.5, 1.5) | 4.60 (d, 5.5) | 4.51 (d, 5.5) | — |
| 8′ | 2.5 (m) | 3.68 (m) | 3.68 (m) | — |
| 9a′ | 3.92 (m) | 3.5 (m) | 3.58 (m) | — |
| 9b′ | 3.61 (m) | 3.4 (m) | 3.45 (dd, | |
| | | | 12.0, 4.0) | |
| 3- | 3.84 (s) | 3.82 (s) | 3.73 (s) | — |
| OCH₃ | | | | |
| 3′- | 3.60 (s) | 3.82 (s) | 3.77 (s) | — |
| OCH₃ | | | | |
| 4′- | 3.82 (s) | — | — | — |
| OCH₃ | | | | |
| 5′- | — | 3.82 (s) | 3.77 (s) | — |
| OCH₃ | | | | |

^{a}NMR data for all compounds acquired at 500 MHZ except 18 which is acquired at 400 MHz

TABLE 4
¹³C-NMR (δ values) Spectroscopic Data for
Compounds 24-26 and 41

| No. | 24 | 25 | 26^{a} | 41 |
|---|---|---|---|---|
| 1 | 126.94 | 132.40 | 133.53 | 122.08 |
| 2 | 113.92 | 110.00 | 111.82 | 114.91 |
| 3 | 147.47 | 147.28 | 148.88 | 145.27 |
| 4 | 145.31 | 145.41 | 147.50 | 151.29 |
| 5 | 114.84 | 114.30 | 115.98 | 114.48 |
| 6 | 123.27 | 119.15 | 121.04 | 122.08 |
| 7 | 41.17 | 72.57 | 74.71 | 198.08 |
| 8 | 78.16 | 86.09 | 89.28 | 74.00 |
| 9 | 178.28 | 60.08 | 61.83 | 64.85 |
| 1′ | 130.93 | 138.50 | 140.20 | — |
| 2′ | 108.45 | 103.80 | 105.20 | — |
| 3′ | 149.36 | 152.89 | 154.15 | — |
| 4′ | 149.55 | 134.50 | 136.50 | — |
| 5′ | 110.82 | 152.89 | 154.15 | — |
| 6′ | 119.80 | 103.80 | 105.20 | — |
| 7′ | 81.46 | 73.74 | 75.22 | — |
| 8′ | 49.88 | 75.90 | 77.45 | — |
| 9′ | 57.19 | 63.00 | 64.33 | — |
| 3-OCH₃ | 54.92 | 55.20 | 56.44 | — |
| 3′-OCH₃ | 54.87 | 54.94 | 56.73 | — |
| 4′-OCH₃ | 55.00 | — | — | — |
| 5′-OCH₃ | — | 54.94 | 56.73 | — |

^{a}NMR data for all compounds acquired at 125 MHz except 3 which is acquired at 100 MHz

Structural Elucidation of MS-EtOAc Compounds.

All of the isolated compounds are identified by examination of their ¹H and/or ¹³C NMR and mass spectral data, and by comparison of these to published literature reports, when available. Table 2 shows the literature references for the known compounds for which previously published NMR data are available and thus these spectral data are not provided here. However, the NMR data for the four new compounds (i.e. 24, 25, 26 and 41), and six of the known compounds (i.e. 31, 33, 44, 46, 47, and 49) which are not available in the literature, are reported here for the first time as follows:

5-(3″,4″-dimethoxyphenyl)-3-hydroxy-3-(4′-hydroxy-3′-methoxybenzyl)-4-hydroxymethyl-dihydrofuran-2-one (24):

colorless amorphous powder; [α]_{D}²⁵+17° (c 1.5 mg/mL, MeOH); (+) HRESIMS, m/z 427.1239 [M+Na]⁺, calcd. for C₂₁H₂₄O₈Na 427.1369; the ¹H and ¹³C-NMR data are shown in Tables 3 and 4, respectively.

(erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol (25):

colorless amorphous powder; [α]_{D}²⁵ 0° (c 0.3 mg/mL, MeOH); (+) HRESIMS, m/z 463.1138 [M+Na]⁺, calcd. for C₂₁H₂₈O₁₀Na 463.1580; the ¹H and ¹³C NMR data are shown in Tables 3 and 4, respectively.

(erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol (26):

colorless amorphous powder; [α]_{D}²⁵+6° (c 2.0 mg/mL, MeOH); (+) HRESIMS, m/z 463.1693 [M+Na]⁺, calcd. for molecular formula O₂₁H₂₈O₁₀Na 463.1580; the ¹H and ¹³C NMR data are shown in Tables 3 and 4, respectively.

2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol (31):

yellowish amorphous powder; (+) HRESIMS, m/z 609.1852 [M+Na]⁺, calcd. for molecular formula C₃₁H₃₈O₁₁; ¹H NMR (CD₃OD, 400 MHz) δ 7.00 (1H, s, H-2), 6.86 (1H, d, J=8.0 Hz, H-6), 6.76 (1H, d, J=8.0 Hz, H-5), 6.74 (4H, s, 6′, 2″, 6″), 5.58 (1H, d, J=6.0 Hz, H-7′), 4.99 (1H, d, J=6.0 Hz, H-7), 4.07 (1H, m, H-8), 3.89 (3H, s, 3″-OCH₃), 3.84 (9H, s, 3, 3′, 5′-OCH₃), 3.80 (2H, m, H-9), 3.58 (2H, t, J=6.4 Hz, H-9″), 3.48 (1H, m, H-8′), 2.64 (2H, t, J=7.6 Hz, H-7″), 1.83 (2H, m, H-8″); ¹³C NMR (CD₃OD, 100 MHz) δ 154.47 (C-3′, 5′), 149.00 (C-3), 147.51 (C-4″), 147.22 (C-4), 145.51 (C-3″), 139.99 (C-1′), 137.51 (C-1″), 137.00 (C-4′), 135.53 (C-1), 129.63 (C-5″), 120.95 (C-6), 118.06 (C-6″), 115.92 (C-5), 114.20 (C-2″), 111.71 (C-2), 103.88 (C-2′, 6′), 89.06 (C-8), 88.65 (C-7′), 88.65 (C-7′), 74.60 (C-7), 65.14 (C-9′), 62.31 (C-9″), 61.85 (C-9), 56.74 (3′, 3, 5′, 7′-OCH₃), 56.41 (3″-OCH₃), 55.95 (C-8′), 36.97 (C-8″), 33.03 (C-7″).

Leptolepisol D (33):

yellowish amorphous powder; (+) HRESIMS, m/z 539.1623 [M+Na]⁺, calcd. for molecular formula C₂₇H₃₂O₁₀; ¹H NMR (CD₃OD, 500 MHz) δ 7.02 (1H, s, H-2), 6.82 (1H, d, J=8.0 Hz, H-6), 6.81 (1H, s, H-2′), 6.74 (1H, d, J=8.0 Hz, H-5), 6.70 (1H, d, J=8.0 Hz, H-6′), 6.68 (1H, s, H-2″), 6.64 (2H, d, J=8.0 Hz, H-5′, 5″), 6.57 (1H, d, J=8.0 Hz, H-6″), 4.93 (1H, d, J=5.5 Hz, H-7′), 4.80 (1H, d, J=5.5 Hz, H-7), 4.30 (1H, m, H-8), 3.86 (1H, m, H-9′a), 3.84 (1H, m, H-9a), 3.82, 3.75, 3.66 (9H, s, 3, 3′, 5′-OCH₃), 3.76 (1H, m, H-9b), 3.70 (1H, m, H-9′a), 2.89 (1H, m, H-8′); ¹³C NMR (CD₃OD, 125 MHz) δ 149.89 (C-3′), 147.29 (C-3), 146.94 (C-3″), 146.64 (C-4′), 145.56 (C-4), 144.80 (C-4″), 137.95 (C-1′), 132.78 (C-1), 130.58 (C-1″), 121.79 (C-6″), 119.46 (C-6), 118.78 (C-2′), 116.90 (C-5), 114.25 (C-5′), 114.22 (C-2″), 113.15 (C-5′), 110.98 (C-6′), 110.40 (C-2), 84.86 (C-8), 73.72 (C-7), 72.62 (C-7′), 62.97 (C-9′), 60.72 (C-9), 55.22 (C-8′), 54.94, 54.93, 54.90 (3, 3′, 5′-OCH₃).

2,3-dihydroxy-1-(3,4-dihydroxyphenyl)-1-propanone (41):

yellowish amorphous powder; [α]_{D}²⁵+267° (c 0.15 mg/ml, MeOH); (−) HRESIMS, m/z 197.0423 [M−H]⁻, calcd. for molecular formula C₉H₉O₅ 197.0450; the ¹H and ¹³C NMR data are shown in Tables 3 and 4, respectively.

Dihydroconiferyl alcohol (44):

white amorphous powder; (+) HRESIMS, m/z 183.1470 [M+H]⁺, calcd. for molecular formula C₁₀H₁₅O₃; ¹H NMR (CD₃OD, 500 MHz) δ 6.76 (1H, s, H-2), 6.69 (1H, d, J=8.0 Hz, H-6), 6.61 (1H, d, J=8.0 Hz, H-5), 3.82 (3H, s, 3-OCH₃), 3.58 (2H, t, J=5.0 Hz, H-9), 2.51 (2H, t, J=7.0 Hz, H-7), 1.78 (2H, m, H-8); ¹³C NMR (CD₃OD, 125 MHz) δ 147.41 (C-3), 144.20 (C-4), 133.53 (C-1), 120.36 (C-6), 114.78 (C-5), 111.74 (C-2), 60.83 (C-9), 34.31 (C-8), 31.24 (C-7).

3′, 4′, 5′-trihydroxyacetophenone (46):

pale yellow amorphous powder; (−) HRESIMS, m/z 167.0409 [M−H]⁻, calcd. for molecular formula C₈H₇O₄; ¹H NMR (CD₃OD, 500 MHz) δ 7.09 (2H, s, H-2, 6), 2.53 (3H, s, CH₃).

3,4-dihydroxy-2-methylbenzaldehyde (47):

pale yellow amorphous powder; (−) HRESIMS, m/z 151.0444 [M−H]⁻, calcd. for molecular formula C₈H₇O₃; ¹H NMR (CD₃OD, 500 MHz) δ 9.96 (1H, s, CHO), 7.27 (1H, d, J=8.0 Hz, H-6), 6.80 (1H, d, J=8.0 Hz, H-5), 2.53 (3H, s, CH₃).

4-(dimethoxymethyl)-pyrocatechol (49):

white amorphous powder; (+) HRESIMS, m/z 183.0999 [M−H]⁻, calcd. for molecular formula C₉H₁₁O₄; ¹H NMR (CD₃OD, 500 MHz) δ 6.84 (1H, s, H-2), 6.75 (2H, s, H-5, 6), 5.23 (1H, s, H-7), 3.30 (6H, s, OCH₃); ¹³C NMR (CD₃OD, 125 MHz) δ 146.81 (C-3), 146.26 (C-4), 131.22 (C-1), 119.57 (C-6), 115.92 (C-5), 114.93 (C-2), 104.95 (C-7), 50.00 (OCH₃).

Analytical HPLC-UV.

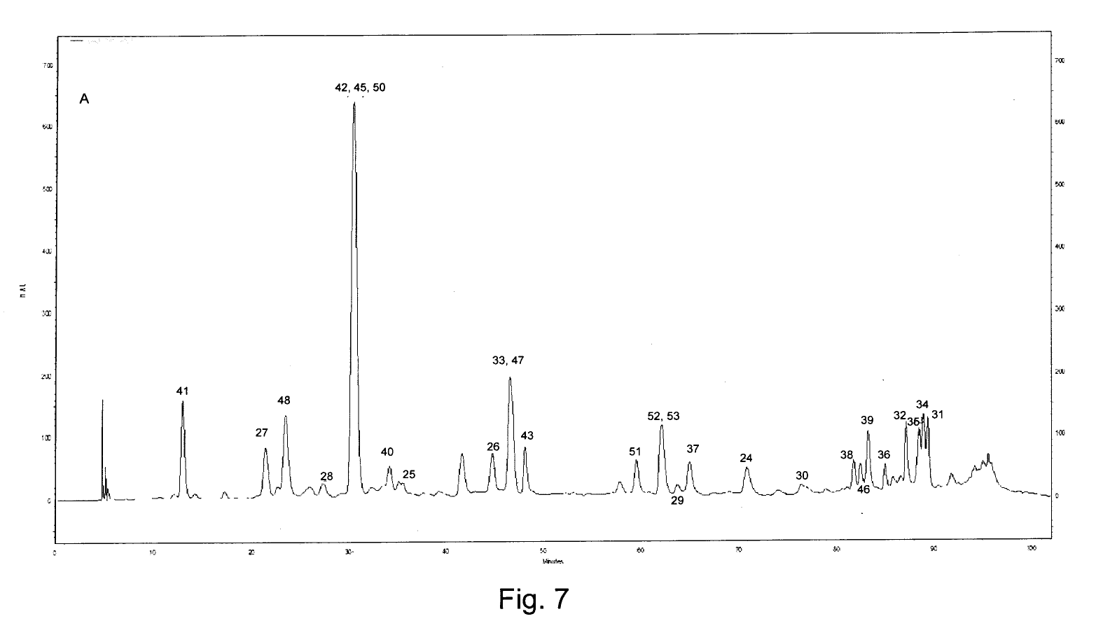

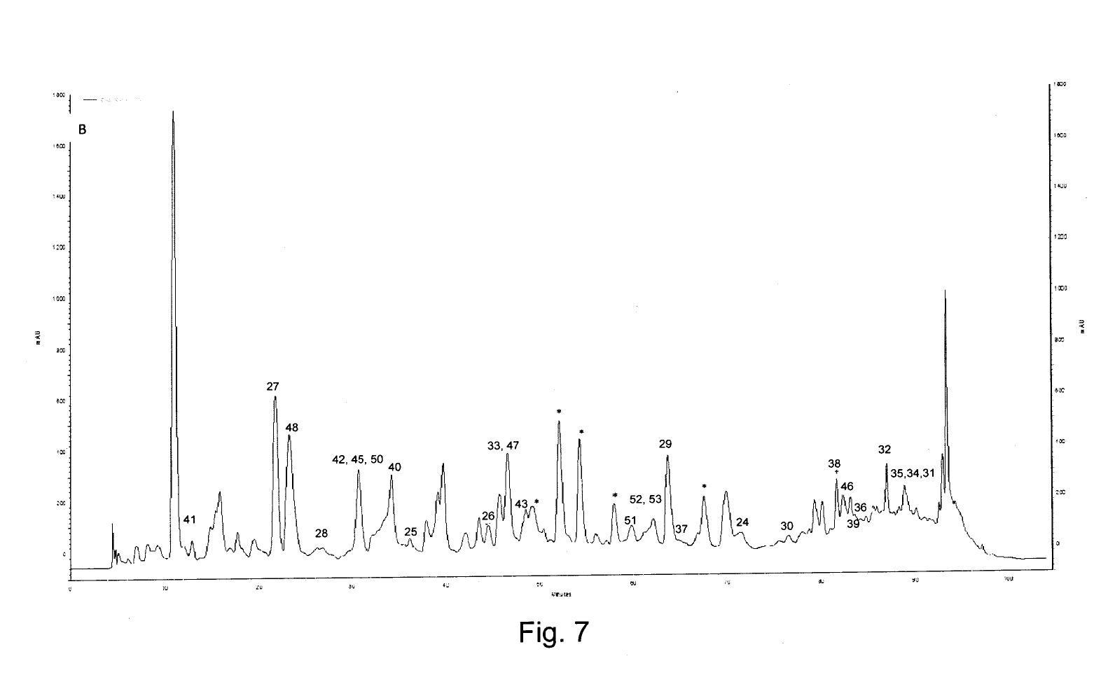

All analyses are conducted on a Luna C18 column (250×4.6 mm i.d., 5 μM; Phenomenex) with a flow rate at 0.75 mL/min and injection volume of 20 μL. A gradient solvent system consisting of solvent A (0.1% aqueous trifluoroacetic acid) and solvent B (methanol) is used as follows: 0-10 min, from 10 to 15% B; 10-20 min, 15% B; 20-40 min, from 15 to 30% B; 40-55 min, from 30 to 35% B; 55-65 min, 35% B; 65-85 min, from 35 to 60% B; 85-90 min, from 60 to 100% B; 90-93 min, 100% B; 93-94 min, from 100 to 10% B; 94-104 min, 10% B. FIG. 7 shows the HPLC-UV chromatograms of all of the isolated compounds (combined into one single injection; 7A) and the total MS-EtOAc extract (50 mg/mL in DMSO; 7B). Unfortunately, due to limited sample quantity, we are not able to include pure compounds 21 and 26 in the HPLC-UV injection shown in FIG. 7A.

Structural Elucidation of Compounds from MS-EtOAc.

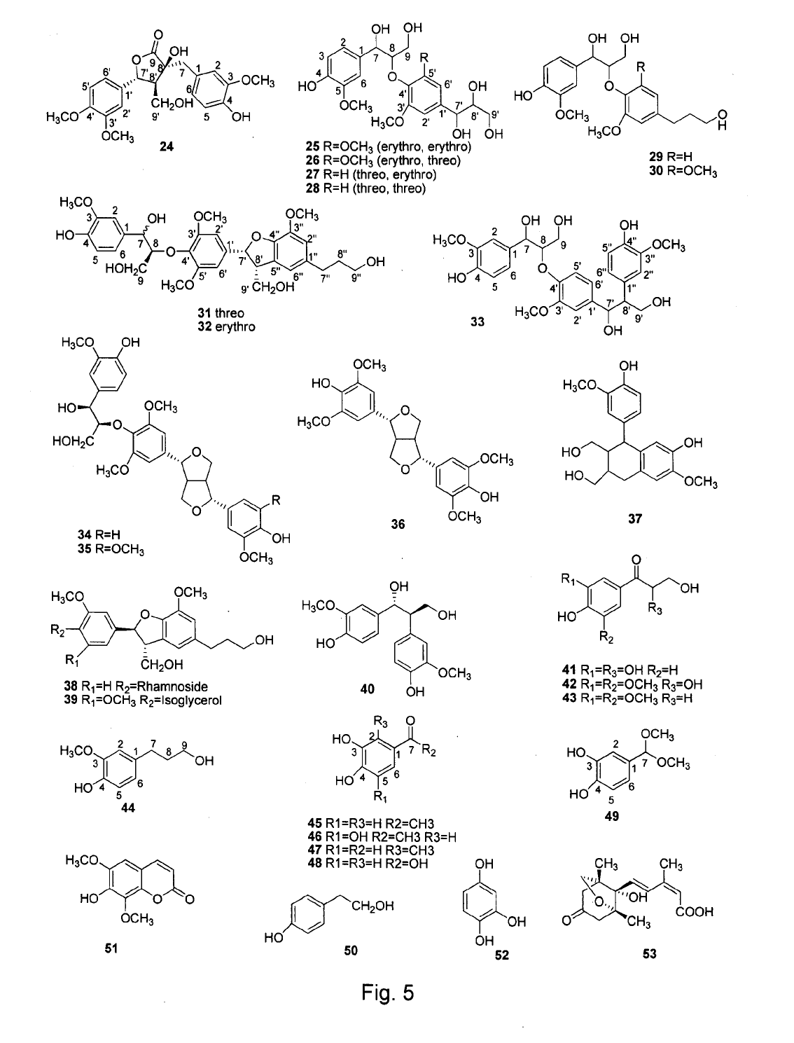

30 compounds are isolated and identified from an ethyl acetate extract of Canadian maple syrup (MS-EtOAc) that have not been previously reported from its butanol extract (MS-BuOH). The structures of the compounds (FIG. 5) are derived through detailed NMR and mass spectral analyses and by comparison of these to literature data when available (see Table 3). FIG. 7A shows the HPLC-UV profile of the 30 compounds isolated from MS-EtOAc, all combined into a single injection, and FIG. 7B shows the chromatogram of the total MS-EtOAc extract.

Four of the isolates are new compounds and thus detailed structural elucidations of these molecules are being reported here for the first time. These are for 3 new lignans (compounds 24-26) and a new phenylpropanoid (compound 41) and are described below.

Elucidation of Compound 24:

Compound 24 is identified as the lignan, 5-(3″,4″-dimethoxyphenyl)-3-hydroxy-3-(4′-hydroxy-3′-methoxybenzyl)-4-hydroxymethyl-dihydrofuran-2-one (1). The ¹H and ¹³C NMR data (Tables 3 and 4, respectively) of compound 24 reveals that it is the aglycon of the known lignan, 3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone previously isolated. The gross structure of 1 is elucidated by comparison of its NMR data to that of its previously reported rhamnosidic form and its structure is confirmed by detailed 2D-NMR analysis and examination of its HRESIMS data: m/z 427.1239 [M+Na]⁺ (calcd. for C₂₁H₂₄O₈Na 427.1369). The rhamnosidic derivative of compound 1 has also been isolated from the hardwood of sugar maple and the relative stereochemistry of that compound is established (Yoshikawa et al, J. Nat. Med. 2010, 65, 191-193). Thus, while we did not determine the absolute stereochemistry of compound 1, we are able to deduce its relative configuration based on comparison of our NOE analyses to that published for its rhamnosidic derivative. The NOEs between H-7′/H9′a, H-7′/H-9′b, H-8′/H-2, H-6, H-2′, and H-6′ indicated the 6-orientations of OH-8 and H-5, and the α-orientation of H-8′. Three methoxyl groups located on two 1,3,4-trisubstituted aromatic rings could also be confirmed at the C-3, C-3′, and C-4′ positions from the NOEs between H-2/OMe (δ 3.84), H-2′/OMe (δ 3.60), and H-5′/OMe (δ 3.82), respectively. Thus, from the above findings, the structure of 24 is deduced as shown in FIG. 5.

Elucidation of Compound 25:

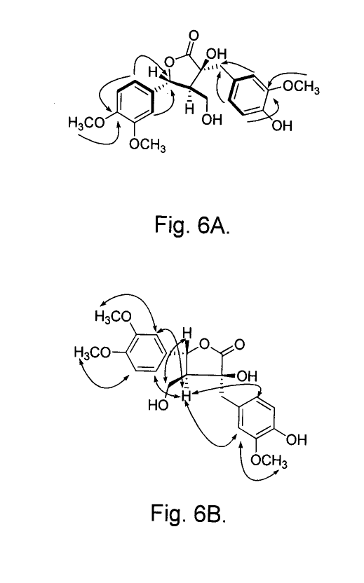

Compound 25 is identified as the lignan, (erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol (25). The positive HRESIMS data exhibited a molecular peak at m/z 463.1138 [M+Na]⁺ (calcd. for C₂₁H₂₈O₁₀Na 463.1580). The ¹H NMR data of 25 (Table 3) indicated the presence of a 1,3,4,5-tetrasubstituted benzene ring [6.75 (2H, s, H-2′, 6′)], a 1,3,4-trisubstituted benzene moiety [δ_{H}: 6.99 (1H, s H-2), 6.74 (1H, d, overlapping, H-5), 6.77 (1H, d, overlapping, H-6)], three methoxyl groups [δ_{H} 3.82 (3,3′,5′-OCH3)], four oxymethines and two oxymethylenes which are all confirmed by the ¹³C NMR data (Table 4). The ¹H-¹H COSY suggested two partial structures, [—CH(OH)CH(O)CH₂OH] and [—CH(OH)CH(OH)CH₂OH]. In the HMBC spectrum (see FIG. 6A), the correlations from δ_{H} 4.91 (1H, d, J=4.5 Hz, H-7) to C-1 (δ 132.40), C-2 (δ 110.0) and C-6 (δ 119.15), from δ_{H} 4.60 (1H, d, J=5.5 Hz, H-7′) to C-1′ (δ 138.50) and C-2′, C-6′ (δ 103.80 equivalent) indicated the presence of one guaiacylglycerol moiety and one syringylglycerol moiety, respectively. Since the C-8 in compound 25 is downfield compared to its C-8′ (δ 86.09 and δ 75.9, respectively), this suggested that the connection of C-8 is to C-4′. This is confirmed by comparison of the ¹³C-NMR data with the known compound 27 which contains one less methoxyl group than compound 25. Therefore, the gross structure of compound 25 is elucidated as 1-[4-[2-hydroxy-2-(4-hydroxy-3-methoxyphenyl)-1-(hydroxymethyl)ethoxy]-3,5-dimethoxyphenyl]-1,2,3-propanetriol. It has been previously reported that for syringoylglycerols and guaiacylglycerol derivatives, the coupling constant (J value) between H-7 and H-8 is ≦5 Hz for the erythro isomer and ≧7 Hz for the threo isomer (29). Thus, the lower coupling constant between H-7 (J=4.5 Hz) and H-7′ (J=5.5 Hz) of compound 25 suggested that it is the erythro,erythro isomer.

Elucidation of Compound 26:

Compound 26 is identified as the lignan, (erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol (26). The positive HRESIMS exhibited a molecular peak at m/z 463.1138 [M+Na]⁺ (calcd. for molecular formula C₂₁H₂₈O₁₀Na 463.1580). The ¹H and ¹³C NMR data of this compound closely resembled that of compound 25 (shown in Tables 3 and 4, respectively). Comparison of the ¹H-NMR spectrum of these two compounds showed that the coupling constant of H-7 (δ 4.89, d, J=7.0 Hz) of compound 26 is greater than that of compound 25 (δ 4.89, d, J=4.5 Hz). From the HPLC-UV analysis (FIG. 7A), it is also evident that compounds 25 and 26 had different retention times under the same chromatographic methods.

It should be noted that the two new lignans isolated, namely compounds 25 and 26, can be regarded as methoxylated derivatives of the known lignans, (threo,erythro)-1-[4-[(2-hydroxy-2-(4-hydroxy-3-methoxyphenyl)-1-(hydroxymethyl)ethoxy]-3-methoxyphenyl]-1,2,3-propanetriol (27), and (threo,threo)-1-[4-[(2-hydroxy-2-(4-hydroxy-3-methoxyphenyl)-1-(hydroxymethyl)ethoxy]-3-methoxyphenyl]-1,2,3-propanetriol (28), respectively, but with different stereochemistry. While the known lignans 27 and 28 have been previously reported from Zantedeschia aethiopica (Della-Grace et al), this is the first report of all four of these compounds in maple syrup (see Table 2). Interestingly, these four lignans elute with distinct retention times under our HPLC conditions (shown in FIG. 7A) which would be useful for future quantification of these compounds in different grades of maple syrup and its products.

Elucidation of Compound 41:

Compound 41 is identified as the phenylypropanoid, 2,3-dihydroxy-1-(3,4-dihydroxyphenyl)-1-propanone (41). The ¹H-NMR data of 41 (see Table 3) indicated the presence of a 1,3,4-trisubstituted benzene moiety [δ_{H}: 7.47 (1H, d, J=8.5 Hz, H-5), 7.45 (1H, s H-2), 6.85 (1H, d, J=8.5 Hz, H-6)] and a —CH(OH)—CH₂OH moiety [5.09 (1H, brs, H-8), 3.88 (1H, d, J=8.0 Hz, H-9a) and 3.73 (1H, m, H-9b)] which is supported by the ¹³C NMR data (Table 4). According to the NMR data, on comparison with compound 20, 3-hydroxy-1-(4-hydroxy-3,5-dimethoxyphenyl)propan-1-one, previously isolated from Ficus beecheyana (20), the H-8 in compound 41 is shifted downfield from δ_{H} 3.20 to 5.09. This indicated that compound 41 is a hydroxyl derivative of compound 43 which is confirmed by the HRESIMS data of m/z 197.0423 suggesting a molecular formula of C₉H₉O₅. It should be noted that the absolute stereochemistry of compound 41 (viz. chiral center at position 8,) is not determined due to limited sample quantity. Thus, further studies would be required to confirm the absolute sterochemistry of compound 41.

Other Compounds.

Apart from the 4 new compounds described above, an additional 26 other compounds are also isolated from MS-EtOAc that have not been previously reported from MS-BuOH. The structures of these compounds are elucidated based on detailed NMR and mass spectral data and by comparison with literature data when available (see Table 10). Since the NMR spectral data for compounds 31, 33, 44, 46, 47, and 49 are not available in the literature, they are being reported here for the first time (provided in the Methods section).

Based on their chemical structures, the 30 isolates from MS-EtOAc can be classified into various phytochemical sub-classes including lignans (24-39), phenylpropanoids (40-44), coumarins (51), simple phenolics (45-50, 52), and a sesquiterpene (53). Among these classes, lignans and phenylpropanoids are the main types of compounds found in MS-EtOAc which is consistent with our earlier findings of MS-BuOH constituents.

It should be noted that this is the first report of 23 of these phenolic compounds, namely, compounds 24-30, 33-35, 37-43, 45-47, 49, 51-52, in maple syrup. However, while phenolic compounds are common to maple syrup, to the best of our knowledge, this is the first published report of a sesquiterpene, namely phaseic acid (53), therein. Phaseic acid is a known oxidative metabolite of the plant hormone, abscisic acid, which has previously been reported from the natural maple sap, and also from Canadian maple syrup. The occurrence of an ABA metabolite in maple syrup is interesting considering that this phytohormone has attracted significant research attention for its efficacy in the treatment of metabolic syndrome, diabetes and inflammation.

Based on the chromatogram shown in FIG. 7B, it is apparent that there are several other peaks at 280 nm characteristic of phenolic compounds in the maple syrup extract. Here it should be noted that apart from the 30 compounds isolated from MS-EtOAc, seven additional compounds are isolated that are previously obtained from MS-BuOH (see FIG. 7B with the marked overlapping peaks). These compounds included erythro-guaiacylglycerol-β-O-4′-dihydroconiferyl alcohol, lyoniresinol, secoisolariciresinol, C-veratroylglycol, scopoletin, vanillin and syringic acid. Also, while not isolated from MS-EtOAc, based on HPLC-UV comparisons with compounds isolated from MS-BuOH, three additional compounds are identified: syringaldehyde, syringenin and (E)-coniferol in MS-EtOAc (data not shown). Thus, apart from the 30 compounds described from MS-EtOAc, an additional 10 compounds previously isolated from MS-BuOH, are also present therein as overlapping compounds (FIG. 7B). Moreover, it should be noted that similar to previous observations, a number of compounds in maple syrup remain un-identified due to low yields and/or degradation of compounds during extraction and isolation procedures.

Identification of a New Compound from the Process of Preparation of Maple Syrup.

According to another embodiment, there is disclosed a new compound from the process of preparation of maple syrup.

Reagents & Materials:

All solvents are either analar or HPLC grade and purchased from Wilkem Scientific Co. (Pawtucket, R.I.). Maple syrup (grade C, 20 L) is provided by the Federation of Maple Syrup Producers of Quebec (Canada). The syrup is kept frozen until extraction when it is subjected to liquid-liquid partitioning with ethyl acetate (10 L×3) followed by n-butanol (10 L×3) solvents, to yield ethyl acetate (4.7 g) and butanol (108 g) extracts, respectively, after solvent removal in vacuo.

Isolation:

A portion of the butanol extract (87 g) is reconstituted in methanol to afford methanol soluble (36 g) and insoluble (57 g) fractions. The methanol soluble fraction is selected for further purification by repeated Sephadex-LH20 column chromatography followed by C 18 semi-preparative HPLC. First, the extract is chromatographed on 65×4 cm Sephadex-LH-20 column eluted with a CH₃OH—H₂O gradient system (3:7 to 1:0, v/v) to afford twelve subfractions, A1-A12. Subfraction A4 (1.6 g) is re-chromatographed on a 65×4 cm Sephadex-LH-20 column eluted with same gradient system (3:7 to 1:0, v/v) to afford twelve subfractions, B1-B12. Subfraction B5 (137.2 mg) is purified by semi-preparative HPLC (Neckman Coulter) using a Waters Sunfire C18 column (250×10 mm i.d., 5 μm, flow=2 mL/min) with a gradient elution system of CH₃OH—H₂O (0.1% trifluoroacetic acid) (1:4, v/v to 1:0, v/v in 60 min) to afford compound 1 (4 mg).

NMR:

Data is collected on a Varian 500 MHz Biospin instrument using CD₃OD as solvent.

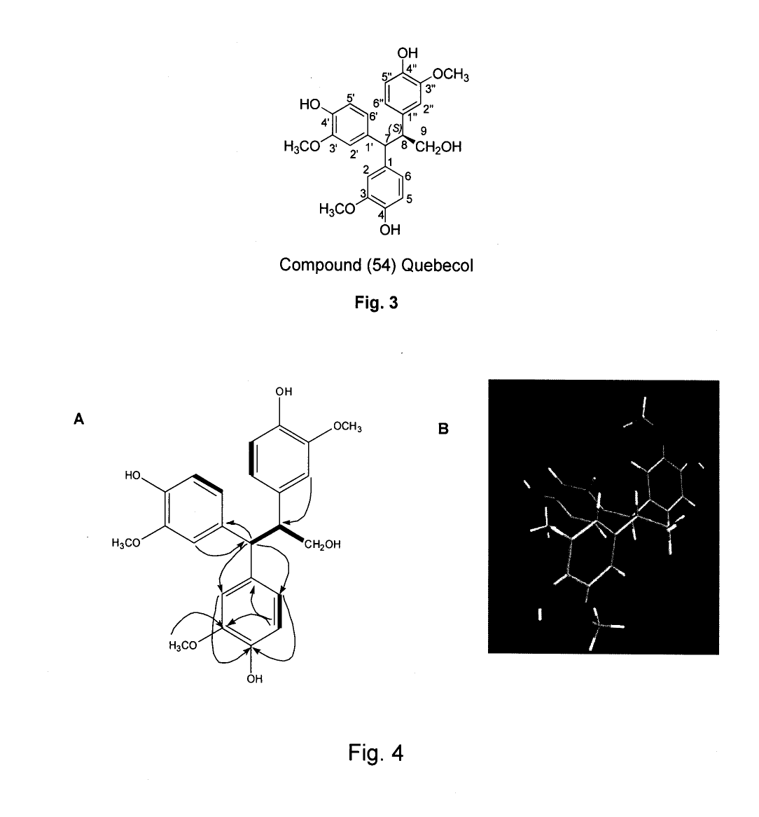

Compound (54)—Quebecol, (FIG. 3) is obtained as pale yellow amorphous powder. The positive ESIMS exhibits a molecular peak at m/z 449.1571 [M+Na]⁺, and negative ESI shows at m/z 425.1979 [M−H]⁻. The ¹H NMR (in DMSO-d₆) spectrum exhibits the signals for three pairs of ABX aromatic system at δ_{H} 6.81 (1H, J=8.0 Hz, H-6), 6.67 (1H, J=8.0 Hz, H-5), 6.98 (1H, s, H-2); 6.56 (1H, J=8.0 Hz, H-6′), 6.41 (1H, J=8.0 Hz, H-5′), 6.78 (1H, s, H-2′); 6.60 (1H, J=8.0 Hz, H-6″), 6.50 (1H, J=8.0 Hz, H-5″), 6.56 (1H, s, H-2″) respectively, suggesting the presence of three benzene rings, which is supported by the ¹³C NMR (in DMSO-d₆) data (Table 5) and ¹H-¹H COSY spectrum analysis (FIG. 4). Three singlet signals at ≡_{H} 3.76, 3.66 and 3.63 with three-proton density for each reveal the presence of three methoxyl groups. Additionally, one doublet signal at δ_{H} 4.02 (1H, J=10.5 Hz, H-7), two multiplet signals at d_{H} 3.41 (1H, m, H-8) and 3.40 (2H, m, H-10) can be observed in the ¹H spectrum. All the proton signals are assigned to the corresponding carbons through direct ¹H-¹³C correlations in the HSQC (Table 5) spectrum, with exception of the two singlets at δ_{H} 8.67 (1H) and 8.43 (2H) which are in good accordance with proton of hydroxyl group. Furthermore a CH—CH—CH2 substructure can be deduced from COSY correlations (FIG. 4) analysis. In the HMBC spectrum, the correlations signals (FIG. 4) from δ_{H} 6.67 (H-5) and 3.76 (3-OCH₃) to C-3 (δ 147.72), δ_{H} 6.41 (H-5′) and 3.66 (3′-OCH₃) to C-3′ (δ 147.17), δ_{H} 6.50 (H-5″) and 3.63 (3″-OCH₃) to C-3″ (δ 147.08), reveals three methoxyl groups substituted on the C-3, 3′and 3″ individually. In the same HMBC experiment, correlation signals show from δ_{H} 4.02 (H-7) to C-2 (112.56), C-6 (120.33) and C-1′ (136.26), and from δ_{H} 6.78 (H-2′) to C-8 (51.42) suggest three benzene rings are attached to the CH—CH—CH₂OH chain on C-7, C-7 and C-8 position respectively.

TABLE 5
¹H and ¹³C NMR data (in DMSO-d6, 500 and 125 MHz)
of compound (54)

| No | δ_{C} | δ_{H} (J in Hz) | No | δ_{C} | δ_{H} (J in Hz) |
|---|---|---|---|---|---|
| 1 | 136.70 | — | 1′ | 136.26 | — |
| 2 | 112.56 | 6.98 (s) | 2′ | 113.15 | 6.78 (s) |
| 3 | 147.72 | — | 3′ | 147.17 | — |
| 4 | 144.92 | — | 4′ | 144.26 | — |
| 5 | 115.72 | 6.67 (d, 8.0) | 5′ | 115.23 | 6.41 (d, 8.0) |
| 6 | 120.33 | 6.81 (d, 8.0) | 6′ | 121.04 | 6.56 (d, 8.0) |
| 7 | 52.67 | 4.02 (d, 10.5) | 1″ | 134.65 | — |
| 8 | 51.42 | 3.41 (m) | 2″ | 113.90 | 6.78 (s) |
| 9 | 64.92 | 3.40 (m) | 3″ | 147.08 | — |
| 3-OCH3 | 56.14 | 3.76 (s) | 4″ | 144.48 | — |
| 3′-OCH3 | 56.01 | 3.66 (s) | 5″ | 115.09 | 6.50 (d, 8.0) |
| 3″-OCH3 | 55.94 | 3.63 (s) | 6″ | 121.77 | 6.60 (d, 8.0) |
| 4-OH | — | 8.64 (s) | 4″-OH | — | 8.43 (s) |
| 4′-OH | — | 8.43 (s) | | | |

The absolute configuration of compound (54) is elucidated by combination of ¹H NMR analysis and computer modelling. The coupling constant of H-7 is 10.5 Hz, suggesting H-7 and H-8 are both at the axial positions, which is in accordance with S configuration. Thus, based on above findings, the structure of compound (54) is elucidated as shown in FIG. 3 to which the common name, quebecol, has been assigned.

Polyphenol Extract from In Vitro Gastrointestinal Digestion

According to another embodiment of the present invention there is disclosed a maple syrup extract subjected to simulated gastrointestinal digestion. Different grades of MS are subjected to in vitro gastrointestinal digestion. The digestion process decreased the phenolic content compared to the initial, non-digested phenolic content. Human colon cancer cell lines (HCT-116, Caco-2) are incubated 4 h daily for 4 days or continuously for 24 h with bioaccessible fractions obtained after the digestion. Maple syrup extracts significantly inhibited cell proliferation in the two experimental conditions due to their high polyphenolic compound content and their synergistic effects.

Maple syrup samples are subjected to successive in vitro gastric and intestinal digestion. Briefly, the samples are digested with a mixture of pepsin-HCl (pH 2.0) for 2 h to simulate gastric digestion, followed by a 2 h intestinal digestion with pancreatin and bile salts (pH 6.5). The digests are centrifuged at 3890 g for 60 min at 4 to separate the soluble fraction (bioaccesible faction) which is pooled. Control samples are run in parallel and consisted of an equivalent volume of cell culture degree water subjected to the same in vitro digestion (mix enzymes+salts). After digestion and to ensure inactivation of enzymes and stability of phenolic compounds, aliquots of the digested samples are acidified to pH 2.0 with formic acid (1.5%), filtered through a 0.45 micron membrane filter Millex-HV13 (Millipore Corp. Bedford, USA) and analyzed using HPLC-MS/MS.

Effects of Maple (Acer) Plant Part Extracts on Proliferation, Apoptosis, and Cell Cycle Arrest of Human Tumorigenic and Non-Tumorigenic Colon Cells

According to another embodiment, there is disclosed extracts from plant parts from Sugar, Red and other maple species, and sugar plant species.

General Experimental Procedures.

Nuclear Magnetic Resonance (NMR) spectra for all compounds are recorded on a Bruker 400 MHz Biospin spectrometer (¹H: 400 MHz, ¹³C: 100 MHz) using deuterated methanol (methanol-d₄) as solvent. Mass Spectral (MS) data are carried out on a Q-Star Elite (Applied Biosystems MDS) mass spectrometer equipped with a Turbo lonspray source and are obtained by direct infusion of pure compounds. High performance liquid chromatography (HPLC) are performed on a Hitachi Elite LaChrom system consisting of a L2130 pump, L-2200 autosampler, and a L-2455 Diode Array Detector all operated by EZChrom Elite software. All solvents are either ACS or HPLC grade and are obtained from Wilkem Scientific (Pawcatuck, R.I.). Unless otherwise stated, all reagents including the MTS salt [3-(4,5-dimethylthiazol-2-yl)-5-(3-carboxymethoxyphenyl)-2-(4-sulfenyl)-2H-tetrazolium salt], gallic acid, Folin-Ciocalteu reagent and etoposide standards are obtained from Sigma-Aldrich.

Plant Materials.

All plant materials are from the Federation of Maple Syrup Producers of Quebec (Canada).

Preparation of Extracts.

Briefly, all plant extracts are enriched for phenolic content by extraction with methanol and prepared using dried and pulverized parts of the harvested plants. For each dried and ground maple plant material (ca. 10.0 g), extractions are performed using methanol (3×100 mL) to afford a dried methanol extract, after solvent removal with a rotary evaporator in vacuo. The dried weights of the extracts obtained from the Sugar and Red maple species are: leaves=0.7 and 3.3 g; twigs/stem=0.3 and 0.69 g, bark=0.85 and 0.80 g; sapwood/heartwood=0.05 and 0.13 g, respectively.

Determination of Total Phenolic Content of Extracts.

The total phenolic contents of the maple extracts are determined according to the Folin-Ciocalteu method and is measured as gallic acid equivalents (GAEs). Briefly, the extracts are diluted 1:100, or as appropriate, with methanol/H₂O (1:1, v/v), and 200 μL of sample is incubated with 3 mL of methanol/H₂O (1:1, v/v) and 200 μL of Folin-Ciocalteau reagent for 10 min at 25° C. After this, 600 μL of a 20% Na₂CO₃ aqueous solution is added to each tube and vortexed. Tubes are further incubated for 20 min at 40° C. After incubation, samples are immediately cooled in an ice bath to room temperature. Samples and standard (gallic acid) are processed identically. The absorbance is determined at 755 nm, and final results are calculated from the standard curve obtained from a Spectramax plate reader.

Analytical HPLC Analyses of the Maple Extracts.

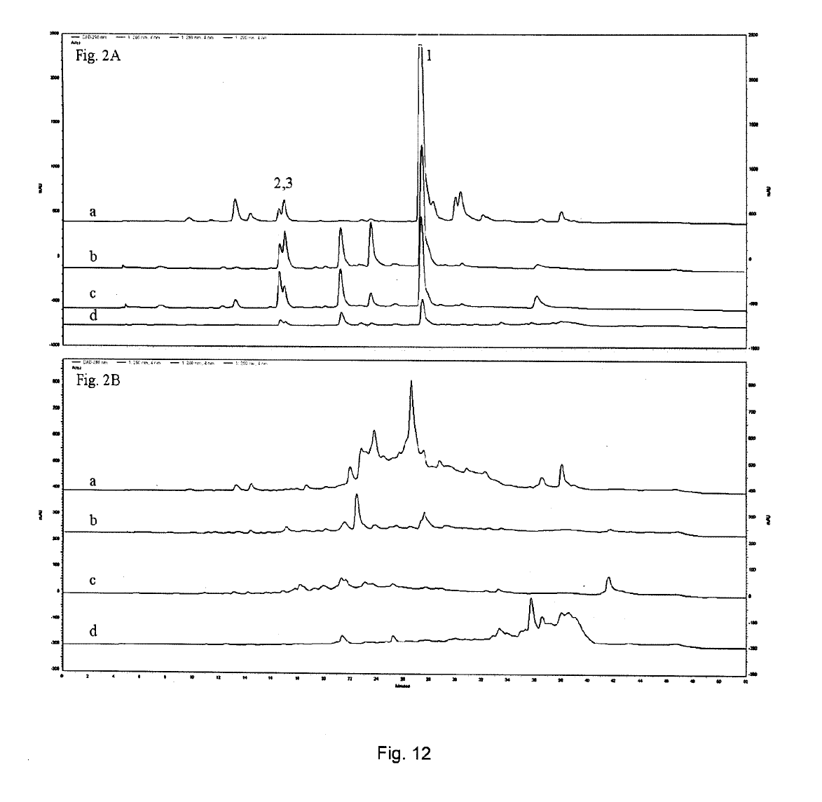

A Luna C18 column (250×4.6 mm i.d., 5 μM; Phenomenex) with a flow rate at 0.75 mL/min and injection volume of 20 μL for all samples (extracts and pure ginnalins-A, B and C) is used. A gradient solvent system consisting of solvent A (0.1% aqueous trifluoroacetic acid) and solvent B (methanol) is used as follows: 0-10 min, from 10 to 15% B; 10-20 min, 15% B; 20-40 min, from 15 to 30% B; 40-55 min, from 30 to 35% B; 55-65 min, 35% B; 65-85 min, from 35 to 60% B; 85-90 min, from 60 to 100% B; 90-93 min, 100% B; 93-94 min, from 100 to 10% B; 94-104 min, 10% B. FIG. 2 shows the HPLC profiles of the maple plant part extracts from the Red maple (FIG. 12A) and Sugar maple (FIG. 12B) species, respectively.

HPLC Standardization of Maple Extracts to Ginnalin-A Content.

A stock solution of 1 mg/mL of a pure standard of ginnalin A (isolated as described below) is prepared in DMSO and then serially diluted to afford samples of 0.5, 0.25, 0.125, 0.0625, 0.03125 mg/mL concentrations, respectively. Each sample is injected in triplicate and a linear six-point calibration curve (r²=0.9997) is constructed by plotting the mean peak area percentage against concentration. Plant extracts are prepared at stock solutions of 2.2 mg/mL in DMSO. All HPLC-UV analyses are carried out with 20 μL injection volumes on a Luna C18 column (250×4.6 mm i.d., 5 μM; Phenomenex) and monitored at a wavelength of 280 nm. A gradient solvent system consisting of solvent A (0.1% aqueous trifluoroacetic acid) and solvent B (methanol, MeOH) is used with a flow rate at 0.75 mL/min as follows: 0-30 min, 10% to 60% B; 30-35 min, 60% to 100% B; 35-40 min, 100% B; 40-41 min, 100% to 10% B; 41-51 min, 100% B. The ginnalin-A concentrations of the maple extracts are quantified based on the standard curve.

Isolation and Identification of Ginnalins-A, B and C.

Air-dried and ground twigs/stems (547 g) of the Red maple species are extracted with methanol (700 mL×3) at room temperature to yield 37 g of dried extract after solvent removal using a rotary evaporator in vacuo. A portion of the dried methanol extract (35 g) is reconstituted in water and subjected to liquid-liquid partitioning sequentially with n-hexanes (500 mL×3), ethyl acetate (500 mL×3) and n-butanol (500 mL×3). The combined butanol extract, after solvent removal in vacuo, yielded 16.1 g of dried extract. A portion of the dried butanol extract (4 g) is chromatographed on a Sephadex-LH-20 column (4.5×64 cm), eluting with a gradient system of methanol/water (7/3 v/v to 100/0 v/v), and then with acetone/water (7/3 v/v). On the basis of analytical HPLC profiles, fourteen combined fractions (Fr. 1-14) are obtained. Ginnalin-A (also known as acertannin, aceritannin, or 2,6-di-O-galloyl-1,5-anhydro-D-glucitol) (70, 306 mg, brown solid) is obtained from Fr. 5 and identified by NMR (¹H and ¹³C) and mass spectral data which corresponded with literature reports. Similarly Fr. 2 (1.55 g), which contained a mixture of ginnalins-B and C is further purified by semipreparative scale HPLC. Briefly, a portion of Fr. 2 (60 mg) is purified on a Waters Sunfire Prep C18 column (250×19 mm i.d., 5 μM) with a gradient solvent system of MeOH/H₂O and flow rate of 2 mL/min. Both ginnalin-B (71, 17 mg, brown solid) and ginnalin-C (72, 15.7 mg, brown solid) are identified by their by ¹H and ¹³C-NMR data which are in agreement with literature.

Preparation of Preparation of a Food-Grade Approved Extract from Maple Syrup.

According to another embodiment of the present invention, there is disclosed a food grade extract from maple tree, including maple tree parts as well as syrup (e.g. Maple Syrup-XAD extract). The generation of the extract requires the utilization of non-food grade solvents and methods, a ‘food-grade approved’ phenolic-enriched extract of maple syrup for future nutraceutical applications is prepared. Towards this end, the maple syrup methanol extract (MS-MeOH) may be prepared using a FDA-food grade resin, such as polymeric resins that include but are not limited to styrene and divinylbenzene resins, and styrene-divinyl-benzene (SDVB) cross-linked copolymer resin. Examples of such resins include but are not limited to Amberlite XAD-4, XAD-2, XAD-7, XAD 7HP, XAD16, XAD16HP, XAD761, XAD1180, XAD1600, XFS-4257, XFS-4022, XUS-40323 and XUS-40322. According to an embodiment of the present invention, the polymeric may be Amberlite XAD-16 (Sigma) and adsorption chromatography is performed by adsorbing the maple syrup on the XAD-16 resin column, eluted with copious amounts of water to remove the natural sugars, then finally eluted with MeOH to yield the maple syrup methanol extract (MS-MeOH) after solvent removal in vacuo. Elution may also be effected with other solvents, which include ethanol.

1. 1 Kg of Amberlite XAD-16 (Sigma) soaked overnight and packed in a large glass column

2. Eluted the XAD-16 column with copious amounts of water.

3. Adsorb a certain volume (to be determined; ca. 500 mL; (make sure it is not over loaded),) of maple syrup which was previously diluted in water so that the solution is not too sticky.

4. Leave maple syrup column on XAD-16 column for ca. 1 h.

5. Elute the column with copious amounts of water to remove sugar (check the eluent for color).

6. Elute with methanol to remove phenolics.

7. Dry the methanol fraction using a rotary evaporator in vacuo, the temperature of the water bath should be set from 37° C. and should not exceed 40° C.

8. The dried sample is maple syrup XAD extract also known as MSX.

9. Repeat the steps to prepare enough quantities.

Preparation of Maple Syrup Butanol Extract without Sugar (MS-BuOH Without Sugar)

According to another embodiment of the present invention, there is disclosed an MS butanol extract without sugar.

1. A known volume of maple syrup (based on the size of your separatory funnel) is subjected to liquid-liquid partitioning with n-butanol (1:1 v/v; 3 times). The maple syrup is diluted with water before partitioning since it is too sticky. (Usually we add around 300 ml water to 1 L maple syrup).

2. Combine the butanol fraction and dry in vacuo as previously described.

3. The dried butanol fraction will be still very sticky and we usually freeze-dry or vacuum dry to make sure it has a powdery consistency

4. The dried butanol extract powder is reconstituted in methanol and the filtered to remove the white solid i.e. sugars. The liquid portion is part is dried in vacuo as previously described.

5. After removing the solvent from the liquid part, add certain methanol to remove sugar again. Repeat filtering and drying.

6. The final dried extract is the MS-BuOH extract without sugar.

7. Repeat steps to prepare enough quantities

Preparation of Maple Syrup Butanol Extract with Sugar (MS-BuOH with Sugar)

According to another embodiment of the present invention, there is disclosed an MS butanol extract without sugar.

Follow steps 1-3 above. In this case, the sugars are not removed with methanol.

Determination of total phenolic content by the Folin-Ciocalteau method

The total phenolic contents of the maple syrup extracts are determined according to the Folin-Ciocalteu method and is measured as gallic acid equivalents (GAEs). Briefly, the extracts were diluted 1:100 with methanol/H₂O (1:1, v/v), and 200 μL of each sample was incubated with 3 mL of methanol/H₂O (1:1, v/v) and 200 μL of Folin-Ciocalteau reagent for 10 min at 25° C. After this, 600 μL of 20% Na₂CO₃ solution was added to each tube and vortexed. Tubes were further incubated for 20 min at 40° C. and after, incubation; samples were immediately cooled in an ice bath to room temperature. Samples and standard (gallic acid) were processed identically. The absorbance was determined at 755 nm, and final results were calculated from the standard curve obtained from a Spectramax plate reader.

Preparation of Red Maple Leaf (RL) Methanol (MeOH) Extract.

According to another embodiment of the present invention, there is disclosed an extract from methanol extraction of red maple leaves. Leaves of Acer rubrum, common name Red-leaf maple, are dried and ground to a fine powder. The powdered plant material is then exhaustively extracted by cold percolation with methanol. Solvent is then removed by a rotary evaporator in vacuo to yield dried extract.

Preparation of Sugar Maple Leaf (SL) Methanol (MeOH) Extract.

According to another embodiment of the present invention, there is disclosed an extract from methanol extraction of sugar maple leaves. Leaves of Acer saccharum, common name sugar maple, are dried and ground to a fine powder. The powdered plant material is then exhaustively extracted by cold percolation with methanol. Solvent is then removed by a rotary evaporator in vacuo to yield dried extract.

Preparation of Stem and Bark Extracts.

According to another embodiment of the present invention, there is disclosed an extract from stem and bark from red and sugar maple. Dried stem or bark plant materials are ground to a fine powder. The powdered plant material is then exhaustively extracted by cold percolation with methanol. Solvent is then removed by a rotary evaporator in vacuo to yield dried extracts.

According to another embodiment, the red maple methanol bark comprises at least four new compounds (55-58):

| No. | Structure | M.W. |
|---|---|---|
| 55 | 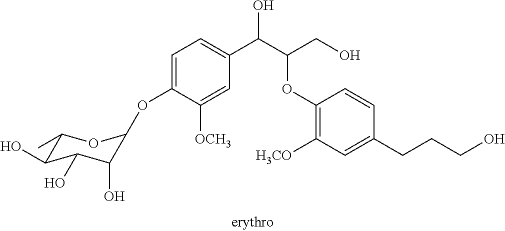 [SMILES: COc1cc(CCCO)ccc1OC(CO)C(O)c1ccc(OC2OC(C)C(O)C(O)C2O)c(OC)c1] | C₂₆H₃₆O₁₁ 524.2258 |
| 56 | 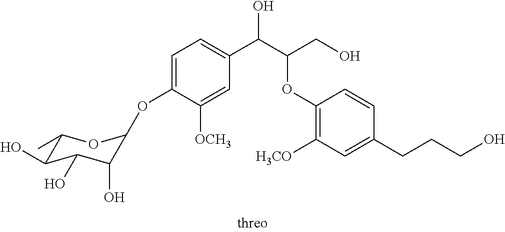 [SMILES: COc1cc(CCCO)ccc1OC(CO)C(O)c1ccc(OC2OC(C)C(O)C(O)C2O)c(OC)c1] | C₂₆H₃₆O₁₁ 524.2258 |
| 57 | 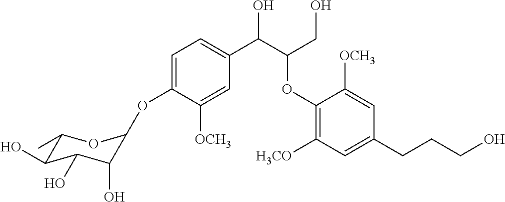 [SMILES: COc1cc(C(O)C(CO)Oc2c(OC)cc(CCCO)cc2OC)ccc1OC1OC(C)C(O)C(O)C1O] | C₂₇H₃₈O₁₂ 554.2363 |
| 58 | 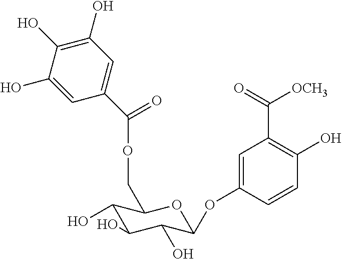 [SMILES: COC(=O)c1cc(OC2OC(COC(=O)c3cc(O)c(O)c(O)c3)C(O)C(O)C2O)ccc1O] | C₂₁H₂₂O₁₃ 482.1060 |

Methods of Preparation of Grade C and D Extracts.

MS-BuOH and MS-EtOAc extracts were prepared as described above from grade C and D maple syrup. Maple syrup of grades C and D are individually partitioned with ethyl acetate to yield ethyl acetate extracts after solvent removal in vacuo. After this, the remaining syrup are then subsequently partitioned with butanol to yield butanol extracts after solvent removal in vacuo.

According to another embodiment of the present invention, the extracts of the present invention may also contain saccharised, such as mono saccharides, disaccharides, trisaccharides, oligosaccharides, and polysaccharides, which include but are not limited to glucose, fructose, galactose, ribose, deoxyribose, mannose, maltose, kojibiose, nigerose, isomaltose, trehalose, β,β-trehalose, α,β-trehalose, sophorose, laminaribiose, gentiobiose, turanose, maltulose, gentiobiulose, mannobiose, melibiose, melibiulose, rutinose, rutinulose, isomaltotriose, nigerotriose, maltotriose, maltotriulose, raffinose, inulin, kestose, nystose, fructosylnystose, bifurcose, a fructooligosaccharide, quebrachitol, arabinogalactan, dextran, inulotriose, inulotetraose.

Methods of Solvent Removal

According to some embodiments, solvent removal from the extracts of the present invention may be effected in vacuo. However, other known techniques may be employed, such as atomization, lyophilization, evaporation, cristallization, dehydratation or any other suitable process to eliminate the aqueous phase from any of the extracts of the present invention.

The present invention will be more readily understood by referring to the following examples which are given to illustrate the invention rather than to limit its scope.

### Example 1

### Anticancer Activity of Maple Extracts and Pure Isolates

A panel of human tumor cell lines by maple extracts and pure isolates is presented.

### Cell Culture

Cell lines included three human colon cancer cells: HT-29 (human colon adenocarcinoma), HCT116 (human colon carcinoma) and Caco-2 (human epithelial colorectal adenocarcinoma). In addition, normal human colon cells are included: CCD-¹⁸Co (human colon fibroblasts). All cell lines are obtained from the American Type Culture Collection (ATCC, Rockville, Md., USA) and maintained at the University of Rhode Island. Caco-2 cells are grown in EMEM medium supplemented with 10% v/v fetal bovine serum, 1% v/v nonessential amino acids, 1% v/v L-glutamine and 1% v/v antibiotic solution (Sigma). HT-29 and HCT-116 cells are grown in McCoy's 5a medium supplemented with 10% v/v fetal bovine serum, 1% v/v nonessential amino acids, 2% v/v HEPES and 1% v/v antibiotic solution. CCD-¹⁸Co cells are grown in EMEM medium supplemented with 10% v/v fetal bovine serum, 1% v/v nonessential amino acids, 1% v/v L-glutamine and 1% v/v antibiotic solution and are used from PDL=26 to PDL=35 for all experiments. Cells are maintained at 37° C. in an incubator under a 5% CO2/95% air atmosphere at constant humidity and maintained in the linear phase of growth. The pH of the culture medium is determined using pH indicator paper (pHydrion™ Brilliant, pH 5.5-9.0, Micro Essential Laboratory, NY, USA) inside the incubator. All of the test samples are solubilized in DMSO (<0.5% in the culture medium) by sonication and are filter sterilised (0.2 μm) prior to addition to the culture media. Control cells are also run in parallel and subjected to the same changes in medium with a 0.5% DMSO.

### Cytotoxicity Assay

The assay is carried out to measure the IC50 values for samples. Briefly, the in vitro cytotoxicity of samples are assessed in tumor cells by a tetrazolium-based colorimetric assay, which takes advantage of the metabolic conversion of MTS [3-(4,5-dimethylthiazol-2-yl)-5-(3-carboxymethoxyphenyl)-2-(4-sulfenyl)-2H-tetrazolium, inner salt] to a reduced form that absorbs light at 490 nm. Cells are counted using a hemacytometer and are plated at 2000-5,000 cells per well, depending on the cell line, in a 96-well format for 24 h prior to drug addition. Test samples and a positive control, etoposide 4 mg/mL (Sigma), are solubilized in DMSO by sonication. All samples are diluted with media to the desired treatment concentration and the final DMSO concentration per well did not exceed 0.5%. Control wells are also included on all plates. Following a 24 h, 48 h or 72 h drug-incubation period at 37° C. with serially diluted test compounds, MTS, in combination with the electron coupling agent, phenazine methosulfate, is added to the wells and cells are incubated at 37° C. in a humidified incubator for 3 h. Absorbance at 490 nm (OD490) is monitored with a spectrophotometer (SpectraMax M2, Molecular Devices Corp., operated by SoftmaxPro v.4.6 software, Sunnyvale, Calif., USA) to obtain the number of surviving cells relative to control populations. The results are expressed as the median cytotoxic concentrations (IC50 values) and are calculated from six-point dose response curves using 4-fold serial dilutions. Each point on the curve is tested in. Data (see tables 6 to 9) are expressed as mean±SE for three replications on each cell line.

TABLE 6
Maple Compounds

| | | | HCT-116 | | HCT-116 | | HT-29 | |
|---|---|---|---|---|---|---|---|---|
| No | URI Code | Name | 48 h(IC50) | SD | 72 h(IC50) | SD | 48 h(IC50) | SD |
| 1 | LL/VIII/43A | [symbol] imethoxy-4,4′-dihydroxy | n.d. | n.d. | n.d. | n.d. | n.d. | n.d. |
| 2 | LL/VIII/49A | Ginnalin B | 66.8 | 1.8 | 50.9 | 2.0 | 98.9 | 1.5 |
| 3 | LL/VIII/23G | Ginnalin C | 86.5 | 1.9 | 70.7 | 2.4 | 107.2 | 2.0 |
| 4 | LL/VIII/58A | 2,3-Dihydro-3-(hydroxymethy [symbol] | 62.8 | 2.0 | 47.4 | 1.3 | 104.7 | 3.1 |
| 5 | LL/VIII/55A | [symbol] xyphenyl-5-(3,4-dimethoxyph [symbol] | n.d. | n.d. | 103.2 | 2.9 | n.d. | n.d. |
| 6 | LL/VIII/55C | Lyoniresinol | n.d. | n.d. | n.d. | n.d. | n.d. | n.d. |
| 7 | LL/VIII/54A | [symbol] -2-[4-(3-hydroxypropyl)-2-met [symbol] | 107.3 | 2.3 | 98.6 | 3.2 | n.d. | n.d. |
| 8 | LL/VIII/56C | [symbol] phenyl)-2-[4-[ [symbol] 1E)-3-hydroxy-1 [symbol] | n.d. | n.d. | n.d. | n.d. | n.d. | n.d. |
| 9 | LL/VIII/56A | 2-Benzenediol (catech [symbol] | 86.0 | 2.4 | 63.0 | 1.3 | 99.4 | 2.1 |
| 10 | Ferulic Acid | Ferulic Acid | 119.1 | 1.4 | 104.5 | 1.7 | 128.2 | 1.1 |
| 11 | p-Coumaric Acid | p-Coumaric Acid | n.d. | n.d. | 124.0 | 1.6 | n.d. | n.d. |
| 12 | Syringic Acid | Syringic Acid | 119.0 | 1.3 | 108.0 | 0.9 | 126.0 | 1.8 |
| 13 | Catechin | Catechin | n.d. | n.d. | n.d. | n.d. | n.d. | n.d. |
| 14 | Epicatechin | Epicatechin | n.d. | n.d. | n.d. | n.d. | n.d. | n.d. |
| 15 | LL/VIII/58C | Vanillin | 120.3 | 1.0 | 109.9 | 1.7 | n.d. | n.d. |
| 16 | LL/VIII/58D | Syringenin | 123.5 | 1.1 | 108.0 | 1.5 | 126.4 | 3.0 |
| 17 | LL/VIII/107A | Catechaldehyde | 63.3 | 1.3 | 52.9 | 1.4 | 71.6 | 1.5 |
| 18 | LL/VIII/107B | Fratexin | 112.0 | 1.8 | 84.5 | 1.6 | 101.1 | 1.2 |
| 19 | LL/VIII/107D | Coniferol | 111.4 | 1.5 | 84.6 | 1.9 | n.d. | n.d. |
| 20 | LL/VIII/109C | [symbol] hydroxy-5-methylphenyl)- [symbol] | 95.8 | 1.6 | 75.0 | 2.7 | 94.6 | 2.5 |
| 21 | LL/VIII/109B | [symbol] 5-methoxy-trans-dihydrogen [symbol] | 78.0 | 2.3 | 59.1 | 1.5 | 88.7 | 2.2 |
| 22 | LL/VIII/108G | Scopoletin | 68.1 | 1.0 | 60.6 | 2.0 | 78.7 | 1.2 |
| 23 | LL/VIII/58E | Syringaldehyde | 66.8 | 1.3 | 56.3 | 1.8 | 90.7 | 3.0 |
| 24 | LL/VIII/10D | Ginnalin A (Aceritanin) | 54.6 | 1.6 | 43.4 | 2.1 | 77.0 | 2.6 |
| 25 | LL/VIII/53A | C-veratroylglicol | 82.9 | 2.1 | 66.7 | 1.5 | 96.0 | 1.5 |
| 26 | LL/VIII/56B | [symbol] xy-3′,4′-dihydroxyacetop [symbol] | n.d. | n.d. | n.d. | n.d. | n.d. | n.d. |
| 27 | Gallic Acid | Gallic Acid | 64.9 | 2.3 | 42.1 | 1.9 | n.d. | n.d. |
| 28 | | Etoposide | 28.5 | 1.9 | 21.9 | 2.5 | 21.2 | 3.4 |

| | HT-29 | | Caco-2 | | Caco-2 | | CCD-18Co | | CCD-18Co | |
|---|---|---|---|---|---|---|---|---|---|---|
| No | 72 h(IC50) | SD | 48 h(IC50) | SD | 72 h(IC50) | SD | 48 h(IC50) | SD | 72 h(IC50) | SD |
| 1 | n.d. | n.d. | n.d. | n.d. | 93.1 | 2.8 | n.d. | n.d. | n.d. | n.d. |
| 2 | 86.5 | 0.6 | 98.7 | 1.9 | 71.6 | 1.9 | n.d. | n.d. | 107.1 | 9.0 |
| 3 | 95.4 | 1.3 | 110.8 | 1.4 | 94.4 | 1.2 | n.d. | n.d. | n.d. | n.d. |
| 4 | 71.6 | 2.8 | 102.7 | 2.8 | 51.8 | 1.9 | n.d. | n.d. | n.d. | n.d. |
| 5 | 98.2 | 1.9 | n.d. | n.d. | 96.8 | 1.6 | n.d. | n.d. | n.d. | n.d. |
| 6 | n.d. | n.d. | n.d. | n.d. | n.d. | n.d. | n.d. | n.d. | n.d. | n.d. |
| 7 | 110.3 | 2.0 | 93.9 | 2.0 | 88.5 | 1.0 | n.d. | n.d. | n.d. | n.d. |
| 8 | n.d. | n.d. | n.d. | n.d. | n.d. | n.d. | n.d. | n.d. | n.d. | n.d. |
| 9 | 70.3 | 2.0 | 71.5 | 2.3 | 64.1 | 2.5 | n.d. | n.d. | 115.0 | 2.7 |
| 10 | 120.7 | 1.3 | n.d. | n.d. | n.d. | n.d. | n.d. | n.d. | n.d. | n.d. |
| 11 | n.d. | n.d. | n.d. | n.d. | n.d. | n.d. | n.d. | n.d. | n.d. | n.d. |
| 12 | 116.3 | 1.8 | n.d. | n.d. | n.d. | n.d. | n.d. | n.d. | n.d. | n.d. |
| 13 | n.d. | n.d. | n.d. | n.d. | n.d. | n.d. | n.d. | n.d. | n.d. | n.d. |
| 14 | n.d. | n.d. | n.d. | n.d. | n.d. | n.d. | n.d. | n.d. | n.d. | n.d. |
| 15 | 113.7 | 1.5 | n.d. | n.d. | n.d. | n.d. | n.d. | n.d. | n.d. | n.d. |
| 16 | 112.9 | 1.5 | n.d. | n.d. | n.d. | n.d. | n.d. | n.d. | n.d. | n.d. |
| 17 | 63.9 | 1.6 | 73.2 | 1.3 | 58.9 | 1.9 | n.d. | n.d. | 126.2 | 4.6 |
| 18 | 93.4 | 2.6 | n.d. | n.d. | 101.2 | 1.3 | n.d. | n.d. | n.d. | n.d. |
| 19 | 113.2 | 1.9 | 115.9 | 2.0 | 96.4 | 2.9 | n.d. | n.d. | n.d. | n.d. |
| 20 | 84.4 | 2.0 | 102.7 | 2.7 | 90.2 | 1.9 | n.d. | n.d. | 147.7 | 6.1 |
| 21 | 68.2 | 1.4 | 85.0 | 3.8 | 70.9 | 2.3 | n.d. | n.d. | 99.6 | 4.9 |
| 22 | 70.2 | 1.3 | 89.3 | 0.9 | 82.0 | 2.0 | n.d. | n.d. | 102.8 | 5.9 |
| 23 | 68.6 | 1.4 | 59.4 | 2.7 | 35.9 | 2.4 | n.d. | n.d. | n.d. | n.d. |
| 24 | 51.6 | 1.3 | 61.6 | 1.8 | 46.5 | 1.0 | n.d. | n.d. | 90.6 | 3.6 |
| 25 | 88.6 | 1.3 | 100.8 | 1.6 | 90.8 | 1.0 | n.d. | n.d. | n.d. | n.d. |
| 26 | n.d. | n.d. | 92.5 | 1.1 | 86.0 | 1.2 | n.d. | n.d. | n.d. | n.d. |
| 27 | n.d. | n.d. | n.d. | n.d. | 89.3 | 1.7 | n.d. | n.d. | n.d. | n.d. |
| 28 | 12.9 | 2.0 | 14.3 | 4.2 | 12.3 | 1.1 | 45.1 | 1.7 | 42.4 | 1.9 |

[symbol]

indicates data missing or illegible when filed

TABLE 7
Maple Extracts

| | | | Concentration | % | HCT-116 | | HCT-116 | |
|---|---|---|---|---|---|---|---|---|
| No | Name | Source | polyphenols (mg/mL) | polyphenols | 48 h(IC50) | SD | 72 h(IC50) | SD |
| 1 | LL/VIII/6F | Sugar maple leaves | 43.796 | 35.0368 | 141.8 | 5.2 | 127.4 | 5.4 |
| 2 | LL/VIII/6D | Red maple leaves (Green) | 56.628 | 45.3024 | 52.6 | 4.7 | 39.8 | 3.4 |
| 3 | LL/VIII/6C | Red maple stem | 63.729 | 50.9832 | 75.6 | 5.1 | 55.7 | 4.0 |
| 4 | LL/VIII/6E | Sugar maple stem | 54.57 | 43.656 | 384.6 | 13.8 | 235.4 | 10.4 |
| 5 | LL/VIII/47B | Grade C butanol | 7.8 | 6.24 | 331.6 | 31.9 | 268.8 | 8.9 |
| 6 | LL/VIII/24B | Grade C EtOAc | 42.625 | 34.1 | 254.0 | 7.4 | 252.4 | 16.4 |
| 7 | LL/VIII/24E | Glade D butanol | 1.3 | 1.04 | 359.8 | 4.3 | 356.6 | 21.8 |
| 8 | LL/VIII/24D | Grade D EtOAc | 37.95 | 30.36 | 166.3 | 7.1 | 148.5 | 17.7 |
| 19 | LL/VIII/141A | Red maple leaves (Fall) | | | 120.1 | 12.1 | 93.8 | 9.4 |
| 21 | LL/VIII/141C | Red maple bark | | | 140.8 | 7.7 | 114.2 | 6.2 |
| 22 | LL/VIII/141D | Red maple heartwood | | | 445.2 | 15.5 | 325.2 | 8.5 |
| | | Etoposide | | | 26.9 | 1.6 | 14.4 | 1.5 |

| | HT-29 | | HT-29 | | Caco-2 | | Caco-2 | | CCD-18Co | | CCD-18Co | |
|---|---|---|---|---|---|---|---|---|---|---|---|---|
| No | 48 h(IC50) | SD | 72 h(IC50) | SD | 48 h(IC50) | SD | 72 h(IC50) | SD | 48 h(IC50) | SD | 72 h(IC50) | SD |
| 1 | 387.0 | 12.7 | 195.1 | 17.6 | 246.1 | 14.2 | 142.5 | 7.5 | n.d. | n.d. | 366.6 | 9.1 |
| 2 | 218.4 | 5.9 | 103.1 | 9.5 | 143.0 | 10.8 | 90.8 | 5.2 | 344.0 | 8.2 | 180.4 | 10.5 |
| 3 | 233.3 | 18.1 | 111.1 | 2.0 | 169.1 | 8.6 | 111.9 | 11.7 | 334.8 | 9.8 | 220.6 | 9.8 |
| 4 | 448.2 | 31.8 | 276.0 | 10.5 | 467.4 | 6.8 | 275.0 | 8.7 | n.d. | n.d. | 406.3 | 12.2 |
| 5 | n.d. | n.d. | 516.3 | 25.0 | n.d. | n.d. | 468.0 | 19.5 | n.d. | n.d. | n.d. | n.d. |
| 6 | 503.8 | 18.9 | 484.4 | 7.6 | 481.5 | 8.8 | 311.6 | 15.4 | n.d. | n.d. | n.d. | n.d. |
| 7 | 507.7 | 22.7 | 486.8 | 21.8 | n.d. | n.d. | 480.5 | 5.8 | n.d. | n.d. | n.d. | n.d. |
| 8 | 419.8 | 17.0 | 308.6 | 7.4 | 315.9 | 13.3 | 204.1 | 5.0 | 438.7 | 14.4 | 323.4 | 9.9 |
| 19 | 152.2 | 11.0 | 124.1 | 9.9 | 134.4 | 12.6 | 107.5 | 4.4 | 352.3 | 11.9 | 250.0 | 9.5 |
| 21 | 214.0 | 15.9 | 145.1 | 13.0 | 159.1 | 9.2 | 122.5 | 6.0 | 372.9 | 11.1 | 297.3 | 8.2 |
| 22 | 489.8 | 12.7 | 331.1 | 18.5 | 477.9 | 13.9 | 451.7 | 9.7 | n.d. | n.d. | 557.8 | 16.7 |
| | 13.4 | 2.2 | 7.3 | 0.8 | 18.9 | 1.6 | 16.1 | 1.9 | 48.7 | 3.4 | 43.9 | 2.2 |

TABLE 8
Maple Extracts

| | | | | HCT- | | HCT- | | | |
|---|---|---|---|---|---|---|---|---|---|
| | | Concetration | | 116 | | 116 | | HT-29 | |
| | | polyphenols | % | 48 h | | 72 h | | 48 h | |
| Number | Source | (mg/mL) | polyphenols | (IC50) | SD | (IC50) | SD | (IC50) | SD |
| 1 | Sugar maple leaves | 43.796 | 35.0368 | 141.8 | 5.2 | 127.4 | 5.4 | 387.0 | 12.7 |
| 2 | Red maple leaves | 56.628 | 45.3024 | 52.6 | 4.7 | 39.8 | 3.4 | 218.4 | 5.9 |
| 3 | Red maple stem | 63.729 | 50.9832 | 75.6 | 5.1 | 55.7 | 4.0 | 233.3 | 18.1 |
| 4 | Sugar maple stem | 54.57 | 43.656 | 384.6 | 13.8 | 235.4 | 10.4 | 448.2 | 31.8 |
| 5 | 4-5 grade C butanol | 7.8 | 6.24 | 331.6 | 31.9 | 268.8 | 8.9 | n.d. | n.d. |
| 6 | Grade C EtOAc | 42.625 | 34.1 | 254.0 | 7.4 | 252.4 | 16.4 | 503.8 | 18.9 |
| 7 | Grade D butanol (no | 1.3 | 1.04 | 359.8 | 4.3 | 356.6 | 21.8 | 507.7 | 22.7 |
| | sugar) | | | | | | | | |
| 8 | Grade D EtOAc | 37.95 | 30.36 | 166.3 | 7.1 | 148.5 | 17.7 | 419.8 | 17.0 |
| 9 | Red maple stem | 48.125 | 38.5 | 95.1 | 3.6 | 60.5 | 2.5 | 170.2 | 11.2 |
| | butanol | | | | | | | | |
| 10 | Red maple stem | 68.125 | 54.5 | 54.4 | 5.5 | 40.7 | 2.5 | 111.2 | 3.7 |
| | EtOAc | | | | | | | | |
| 11 | Red maple stem | 49.975 | 39.98 | 92.9 | 3.5 | 56.2 | 7.0 | 161.0 | 9.7 |
| | Methanol | | | | | | | | |
| 12 | Norway maple stem | 22.7 | 18.16 | 237.4 | 6.4 | 142.6 | 6.2 | 273.7 | 14.7 |
| 13 | Sugar maple leaves | 42.316 | 33.8528 | 138.0 | 5.2 | 126.9 | 3.0 | 264.2 | 8.9 |
| 14 | Sugar maple stem | 23.414 | 18.7312 | 482.7 | 21.5 | 415.4 | 20.0 | 699.2 | 42.0 |
| 15 | Red maple leaves fall | 58.9 | 47.12 | 80.0 | 5.3 | 54.8 | 2.8 | 164.0 | 6.1 |
| 16 | Sugar maple leaves | 49.574 | 39.6592 | 190.4 | 13.7 | 97.2 | 20.8 | 277.5 | 17.9 |
| | fall | | | | | | | | |
| 17 | Red maple stem | 56.457 | 45.1656 | 71.7 | 2.3 | 44.7 | 2.1 | 130.4 | 11.0 |
| 18 | Red maple leaves | 61.846 | 49.4768 | 80.9 | 5.3 | 58.7 | 0.6 | 143.5 | 8.2 |
| | (green) | | | | | | | | |
| 19 | Red maple leaves | | | 120.1 | 12.1 | 93.8 | 9.4 | 152.2 | 11.0 |
| | (Canada) fall | | | | | | | | |
| 20 | Red maple fruit | | | 429.2 | 11.3 | 291.4 | 13.8 | 471.8 | 9.1 |
| | (USA) | | | | | | | | |
| 21 | Red maple bark | | | 140.8 | 7.7 | 114.2 | 6.2 | 214.0 | 15.9 |
| | (Canada) | | | | | | | | |
| 22 | Red maple heatwood | | | 445.2 | 15.5 | 325.2 | 8.5 | 489.8 | 12.7 |
| 23 | Sugar maple bark | | | 168.0 | 9.4 | 128.0 | 9.4 | 222.5 | 10.0 |
| | (MeOH) | | | | | | | | |
| 24 | Sugar maple bark | | | 127.8 | 2.6 | 77.3 | 3.0 | 146.8 | 3.1 |
| | (MeOH) | | | | | | | | |
| 25 | Sugar maple bark | | | 395.3 | 4.9 | 227.8 | 4.0 | 404.4 | 4.4 |
| | (Ethylacetate) | | | | | | | | |
| 26 | Sugar maple bark | | | 65.0 | 3.3 | 46.9 | 3.5 | 76.8 | 4.3 |
| | (ButOH) | | | | | | | | |
| 27 | Sugar maple | | | 303.4 | 6.3 | 292.4 | 6.0 | 334.0 | 5.6 |
| | heatwood | | | | | | | | |
| | Etoposide | | | 26.9 | 1.6 | 14.4 | 1.5 | 13.4 | 2.2 |

| | | | | | | | CCD- | | CCD- | |
|---|---|---|---|---|---|---|---|---|---|---|
| | HT-29 | | Caco-2 | | Caco-2 | | 18Co | | 18Co | |
| | 72 h | | 48 h | | 72 h | | 48 h | | 72 h | |
| Number | (IC50) | SD | (IC50) | SD | (IC50) | SD | (IC50) | SD | (IC50) | SD |
| 1 | 195.1 | 17.6 | 246.1 | 14.2 | 142.5 | 7.5 | n.d. | n.d. | 366.6 | 9.1 |
| 2 | 103.1 | 9.5 | 143.0 | 10.8 | 90.8 | 5.2 | 344.0 | 8.2 | 180.4 | 10.5 |
| 3 | 111.1 | 2.0 | 169.1 | 8.6 | 111.9 | 11.7 | 334.8 | 9.8 | 220.6 | 9.8 |
| 4 | 276.0 | 10.5 | 467.4 | 6.8 | 275.0 | 8.7 | n.d. | n.d. | 406.3 | 12.2 |
| 5 | 516.3 | 25.0 | n.d. | n.d. | 468.0 | 19.5 | n.d. | n.d. | n.d. | n.d. |
| 6 | 484.4 | 7.6 | 481.5 | 8.8 | 311.6 | 15.4 | n.d. | n.d. | n.d. | n.d. |
| 7 | 486.8 | 21.8 | n.d. | n.d. | 480.5 | 5.8 | n.d. | n.d. | n.d. | n.d. |
| 8 | 308.6 | 7.4 | 315.9 | 13.3 | 204.1 | 5.0 | 438.7 | 14.4 | 323.4 | 9.9 |
| 9 | 113.2 | 6.7 | 168.6 | 10.5 | 115.8 | 8.2 | 267.5 | 11.3 | 224.9 | 10.6 |
| 10 | 73.3 | 6.8 | 102.8 | 10.5 | 66.1 | 3.7 | 180.8 | 8.4 | 115.6 | 7.1 |
| 11 | 101.9 | 11.7 | 146.7 | 9.5 | 95.5 | 6.9 | 264.0 | 10.9 | 135.8 | 7.8 |
| 12 | 202.7 | 7.4 | 276.1 | 9.3 | 176.4 | 7.1 | 362.6 | 10.4 | 244.3 | 8.5 |
| 13 | 218.9 | 8.1 | 254.1 | 6.2 | 141.5 | 8.0 | 328.1 | 9.0 | 294.8 | 8.9 |
| 14 | 411.0 | 14.3 | 492.1 | 21.6 | 437.9 | 13.0 | n.d. | n.d. | n.d. | n.d. |
| 15 | 119.2 | 10.9 | 159.1 | 5.9 | 90.1 | 9.6 | 242.9 | 10.7 | 208.7 | 5.3 |
| 16 | 129.5 | 12.6 | 247.3 | 8.2 | 117.6 | 8.3 | 336.7 | 9.5 | 228.1 | 11.0 |
| 17 | 86.4 | 2.7 | 99.0 | 7.4 | 63.5 | 4.5 | 196.2 | 5.1 | 118.0 | 6.3 |
| 18 | 105.8 | 11.8 | 128.1 | 4.7 | 84.4 | 7.7 | 205.9 | 6.0 | 143.8 | 7.1 |
| 19 | 124.1 | 9.9 | 134.4 | 12.6 | 107.5 | 4.4 | 352.3 | 11.9 | 250.0 | 9.5 |
| 20 | 309.3 | 10.8 | 461.9 | 12.3 | 391.5 | 10.3 | n.d. | n.d. | 462.6 | 12.3 |
| 21 | 145.1 | 13.0 | 159.1 | 9.2 | 122.5 | 6.0 | 372.9 | 11.1 | 297.3 | 8.2 |
| 22 | 331.1 | 18.5 | 477.9 | 13.9 | 451.7 | 9.7 | n.d. | n.d. | 557.8 | 16.7 |
| 23 | 145.5 | 8.6 | 119.2 | 5.1 | 97.8 | 2.2 | n.d. | n.d. | 431.8 | 9.3 |
| 24 | 81.5 | 3.5 | 124.8 | 4.0 | 75.7 | 2.5 | n.d. | n.d. | 332.9 | 6.1 |
| 25 | 253.7 | 5.3 | 398.1 | 4.6 | 209.5 | 7.1 | n.d. | n.d. | 379.6 | 4.1 |
| 26 | 52.5 | 2.3 | 63.1 | 2.0 | 48.9 | 1.6 | 195.2 | 5.3 | 165.9 | 6.0 |
| 27 | 306.2 | 4.9 | 295.9 | 8.6 | 271.3 | 6.7 | n.d. | n.d. | n.d. | n.d. |
| | 7.3 | 0.8 | 18.9 | 1.6 | 16.1 | 1.9 | 48.7 | 3.4 | 43.9 | 2.2 |

TABLE 9
Maple Extracts

| | HCT-116 | | | | HT-29 | | | |
|---|---|---|---|---|---|---|---|---|
| | 48 h | | 72 h | | 48 h | | 72 h | |
| Source | IC50 (ppm)^{a} | % Viability^{b} | IC50 (ppm)^{a} | % Viability^{b} | IC50 (ppm)^{a} | % Viability^{b} | IC50 (ppm)^{a} | % Viability^{b} |
| Sugar maple leaves | 141.8 ± 5.2 | 94.9 ± 0.9 | 127.4 ± 5.4 | 94.8 ± 2.3 | 387.0 ± 12.7 | 93.8 ± 0.8 | 195.1 ± 17.6 | 91.8 ± 1.6 |
| Red maple leaves | 52.6 ± 4.7 | 95.8 ± 0.5 | 39.8 ± 3.4 | 94.6 ± 2.9 | 218.4 ± 5.9 | 94.9 ± 1.1 | 103.1 ± 9.5 | 93.2 ± 1.4 |
| Red maple stem | 75.6 ± 5.1 | 96.8 ± 0.9 | 55.7 ± 4.0 | 94.7 ± 1.1 | 233.3 ± 18.1 | 94.3 ± 0.3 | 111.1 ± 2.0 | 92.2 ± 2.92 |
| Sugar maple stem | 384.6 ± 13.8 | 95.0 ± 1.8 | 235.4 ± 10.4 | 96.9 ± 0.1 | 448.2 ± 31.8 | 93.8 ± 1.8 | 276.0 ± 10.5 | 92.1 ± 3.8 |
| Red maple fruit | 429.2 ± 11.3 | 98.0 ± 1.8 | 291.4 ± 13.8 | 98.6 ± 1.4 | 471.8 ± 9.1 | 94.9 ± 1.4 | 309.3 ± 10.8 | 92.9 ± 1.8 |
| Red maple bark | 140.8 ± 7.7 | 96.8 ± 1.3 | 114.2 ± 6.2 | 97.9 ± 0.9 | 214.0 ± 15.9 | 95.7 ± 1.1 | 145.1 ± 13.0 | 94.4 ± 1.6 |
| Red maple heartwood | 445.2 ± 15.5 | 95.5 ± 1.1 | 325.2 ± 8.5 | 96.3 ± 1.3 | 489.8 ± 12.7 | 97.2 ± 1.8 | 331.1 ± 18.5 | 91.1 ± 1.1 |
| Sugar maple bark | 127.8 ± 2.6 | 98.0 ± 1.6 | 77.3 ± 3.0 | 97.4 ± 1.6 | 146.8 ± 3.1 | 97.5 ± 1.8 | 81.5 ± 3.5 | 94.5 ± 2.2 |
| Sugar maple | 303.4 ± 6.3 | 98.7 ± 1.2 | 292.4 ± 6.0 | 97.6 ± 1.1 | 334.0 ± 5.6 | 97.2 ± 2.0 | 306.2 ± 4.9 | 97.2 ± 1.3 |
| heartwood | | | | | | | | |
| | Caco-2 | | | | CCD-18Co | | | |
| | 48 h | | 72 h | | 48 h | | 72 h | |
| Source | IC50 (ppm)^{a} | % Viability^{b} | IC50 (ppm)^{a} | % Viability^{b} | IC50 (ppm)^{a} | % Viability^{b} | IC50 (ppm)^{a} | % Viability^{b} |
| Sugar maple leaves | 246.1 ± 14.2 | 95.1 ± 1.3 | 142.5 ± 7.5 | 95.1 ± 1.1 | n.d. | 92.8 ± 1.7 | 366.6 ± 9.1 | 98.0 ± 2.0 |
| Red maple leaves | 143.0 ± 10.8 | 94.3 ± 1.4 | 90.8 ± 5.2 | 97.0 ± 1.4 | 344.0 ± 8.2 | 97.5 ± 1.1 | 180.4 ± 10.5 | 95.0 ± 0.5 |
| Red maple stem | 169.1 ± 8.6 | 96.5 ± 0.7 | 111.9 ± 11.7 | 95.8 ± 1.8 | 334.8 ± 9.8 | 94.0 ± 0.7 | 220.6 ± 9.8 | 96.3 ± 1.9 |
| Sugar maple stem | 467.4 ± 6.8 | 91.9 ± 0.8 | 275.0 ± 8.7 | 95.5 ± 0.8 | n.d. | 94.2 ± 2.1 | 406.3 ± 12.2 | 93.7 ± 0.7 |
| Red maple fruit | 461.9 ± 12.3 | 97.5 ± 0.2 | 391.5 ± 10.3 | 94.8 ± 2.0 | n.d. | 96.6 ± 1.7 | 462.6 ± 12.3 | 92.9 ± 1.0 |
| Red maple bark | 159.1 ± 9.2 | 94.2 ± 1.2 | 122.5 ± 6.0 | 96.5 ± 1.5 | 372.9 ± 11.1 | 98.0 ± 1.3 | 297.3 ± 8.2 | 94.4 ± 1.2 |
| Red maple heartwood | 477.9 ± 13.9 | 96.0 ± 1.5 | 451.7 ± 9.7 | 97.8 ± 1.2 | n.d. | 97.2 ± 1.6 | 557.8 ± 16.7 | 98.0 ± 1.8 |
| Sugar maple bark | 124.8 ± 4.0 | 93.1 ± 2.1 | 75.7 ± 2.5 | 96.2 ± 1.3 | n.d. | 96.8 ± 1.7 | 332.9 ± 6.1 | 96.8 ± 1.7 |
| Sugar maple | 295.9 ± 6.6 | 95.4 ± 2.2 | 271.3 ± 6.7 | 95.4 ± 1.3 | n.d. | 97.3 ± 1.7 | n.d. | 98.1 ± 2.1 |
| heartwood | | | | | | | | |

### Example 2

### Antioxidant Assay

Antioxidant Assay.

The antioxidant potential of the Canadian maple syrup ethyl acetate extract (MS-EtOAc) and the pure compounds are determined on the basis of the ability to scavenge the DPPH radical. The DPPH radical scavenging activity of ascorbic acid (vitamin C) and the synthetic commercial antioxidant, butylated hydroxytoluene (BHT) are also assayed as positive controls (see Table 10). The assay is conducted in a 96-well format using serial dilutions of 100 μL aliquots of test compounds (ranging from 2500 to 26 μg/mL), ascorbic acid (1000-10.4 μg/mL), and BHT (250,000-250 μg/mL). After this, DPPH (150 μL) is added to each well to give a final DPPH concentration of 137 μM. Absorbance is determined after 30 min at 515 nm, and the scavenging capacity (SC) is calculated as SC %=[(A0−A1/A0)]×100, where A0 is the absorbance of the reagent blank and A1 is the absorbance of the test samples. The control contained all reagents except the compounds, and all tests are performed in triplicate. IC₅₀ values denote the concentration of sample required to scavenge 50% DPPH free radicals.

TABLE 10
Antioxidant Activities of Pure Compounds Isolated from an Ethyl
Acetate Extract of Canadian Maple Syrup Showing 50%
Inhibitory Concentrations (IC₅₀) in the Diphenylpicrylhydrazyl
(DPPH) Radical Scavenging Assay.^{a}

| No. | IC₅₀ (μM) | No. | IC₅₀ (μM) |
|---|---|---|---|
| 1 | 946.37 ± 58.5 | 18 | 111.78 ± 5.1 |
| 2 | 1540.91 ± 0.5 | 19^{a} | 258.40 ± 33.8 |
| 3 | 925 ± 179.0 | 20 | 321.53 ± 31.9 |
| 7 | 740.20 ± 3.4 | 22 | 138.16 ± 28.2 |
| 8 | 655.29 ± 14.4 | 23 | 10125 ± 1668.0 |
| 9 | 478.95 ± 42.1 | 24 | 254.17 ± 32.5 |
| 10 | 578.49 ± 1.3 | 25 | 97.83 ± 24.0 |
| 11 | 422.94 ± 2.4 | 27 | 163.93 ± 15.2 |
| 12 | 207.93 ± 41.3 | 28 | 813.81 ± 37.7 |
| 13 | 68.90 ± 5.7 | 29 | 139.42 ± 13.3 |
| 14 | 694.44 ± 110.2 | 30^{b} | 903.57 |
| 15 | 1810.28 ± 265.6 | Ascorbic | 40.23 ± 13.4 |
| | | acid | |
| 16 | 2876.44 ± 44.0 | BHT | 3000.98 ± 1122.2 |
| 17 | 703.12 ± 141.4 | | |

^{a}values are mean ± Standard deviation.
^{b}Only tested once because of the limited sample quantity.
BHT, a synthetic commercial antioxidant, butylated hydroxytoluene.
Because of limited sample quantity all compounds are evaluated except 27, 28, 29, 44 and 49.

The assay is conducted in a 96-well format using serial dilutions of 100 μL aliquots of test compounds (ranging from 2500-26 μg/mL), ascorbic acid (1000-10.4 μg/mL), and BHT (250,000-250 μg/mL). Then DPPH (150 μL) is added to each well to give a final DPPH concentration of 137 μM. Absorbance is determined after 30 min at 515 nm, and the scavenging capacity (SC) is calculated as SC %=[(A0−A1/A0)]×100 where A0 is the absorbance of the reagent blank, and A1 is the absorbance with test samples. The control contained all reagents except the compounds and all tests are performed in triplicate. IC₅₀ values denote the concentration of sample required to scavenge 50% DPPH free radicals.

Vitamin C and BHT showed IC₅₀ values of 40 μM (ca. 7.08 μg/mL) and 3000 μM (ca. 660 μg/mL), respectively, and the antioxidant activity of the MS-EtOAc (IC₅₀=77.5 μg/mL) and several of the pure isolates are comparable to vitamin C and superior to BHT.

In summary, 30 compounds are isolated from MS-EtOAc that have not been previously reported. Among these, four of the isolates are new compounds and 24 others are being reported from maple syrup for the first time. In addition, MS-EtOAc contains 10 additional/overlapping compounds that are also present in MS-BuOH. The results reported here advances current knowledge of maple syrup constituents and confirm that this plant derived natural sweetener contains a wide diversity of phytochemicals, among which phenolic compounds predominate. Thus, the biological properties of these maple syrup constituents may impart potential health benefits to this natural sweetener.

### Example 3

### Effects of Maple Syrup Extracts and their Phenolic Constituents on Proliferation, Apoptosis, and Cell Cycle Arrest of Human Tumorigenic and Non-Tumorigenic Colon Cells

Chemicals and General Experimental Procedures

All solvents are either ACS or HPLC grade and are obtained from Wilkem Scientific (Pawcatuck, R.I., USA). Unless otherwise stated, all reagents including the MTS salt [3-(4,5-dimethylthiazol-2-yl)-5-(3-carboxymethoxyphenyl)-2-(4-sulfenyl)-2H-tetrazolium salt], the Folin-Ciocalteau reagent, and the chemotherapeutic drug, etoposide, are obtained from Sigma-Aldrich. High performance liquid chromatography (HPLC) is performed on a Hitachi Elite LaChrom system consisting of a L2130 pump, L-2200 autosampler, and a L-2455 Diode Array Detector all operated by EZChrom Elite software.

Preparation of Phenolic-Enriched Maple Syrup Extracts

Maple syrup is a 66° Brix syrup which contains sucrose as its predominant sugar. Thus, the phenolic-enriched extracts of maple syrup are prepared using the methods described above. The organic extracts of maple syrup having different phenolic profiles are prepared (i.e. quantitative and qualitative differences) for biological evaluation in the anticancer assays. Thus, a combination of solvent-solvent partitioning using the organic solvents, ethyl acetate (EtOAc) and butanol (BuOH), as well as adsorption XAD-16 resin chromatography, using methanol (MeOH) as eluent, are utilized for the extraction of two of the darkest grades (C and D) of maple syrup (further described below).

A description of the methodology used for the organic solvent extractions of maple syrup is described above. Briefly, both grades of maple syrup (provided by the Federation of Maple Syrup Producers of Quebec, Canada) are shipped frozen to our laboratory, and stored at −20° C. until extraction. Aliquots of each grade of maple syrup are individually subjected to sequential liquid-liquid partitioning with EtOAc followed by BuOH to yield maple syrup ethyl acetate (MS-EtOAc) and maple syrup butanol (MS-BuOH) extracts, respectively, after solvent removal with a rotary evaporator in vacuo. Apart from these two extracts (i.e. MS-EtOAc and MS-BuOH), the generation of which required the utilization of non-food grade solvents and methods, a ‘food-grade approved’ phenolic-enriched extract of maple syrup for future nutraceutical applications is prepared. Towards this end, the maple syrup methanol extract (MS-MeOH) is prepared using a FDA-food grade resin (Amberlite XAD-16; Sigma) adsorption chromatography by adsorbing the maple syrup on the XAD-16 resin column, eluted with copious amounts of water to remove the natural sugars, then finally eluted with MeOH to yield the maple syrup methanol extract (MS-MeOH) after solvent removal in vacuo. All of the extracts are standardized to phenolic content (by the Folin-Ciocalteau method) and evaluated for phenolic constituents by HPLC-UV analyses as described below.

Isolation and Identification of Pure Compounds and HPLC Analyses

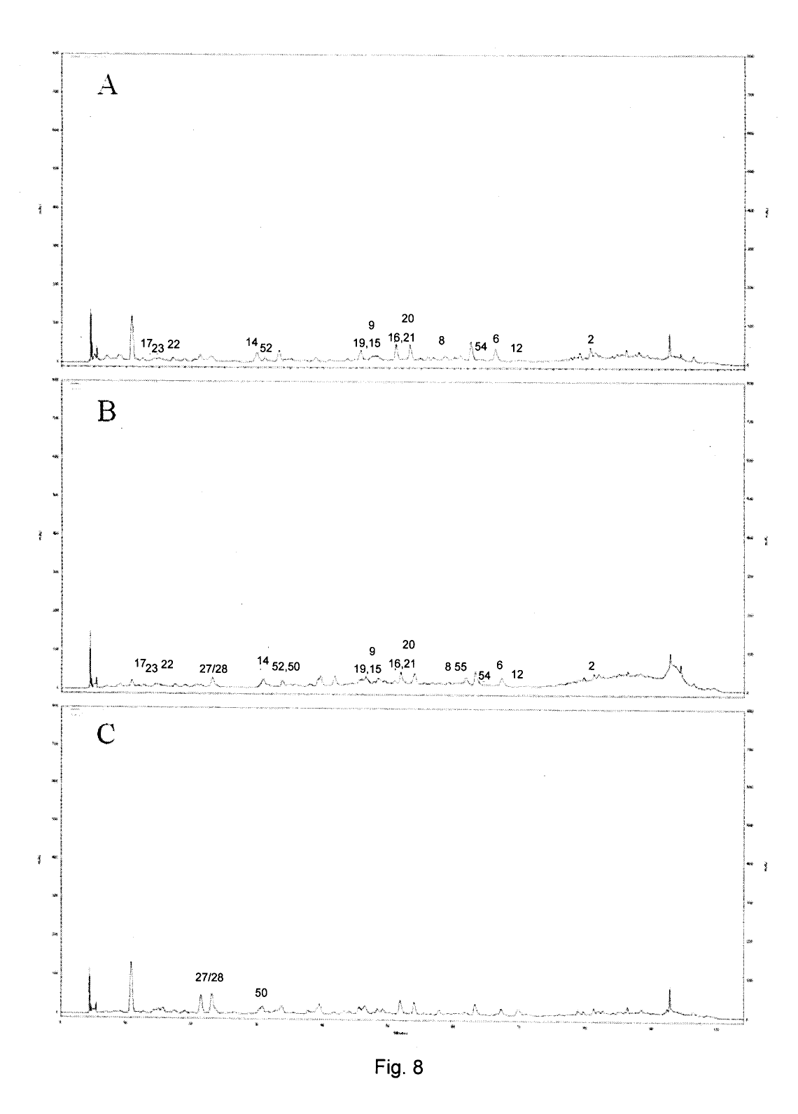

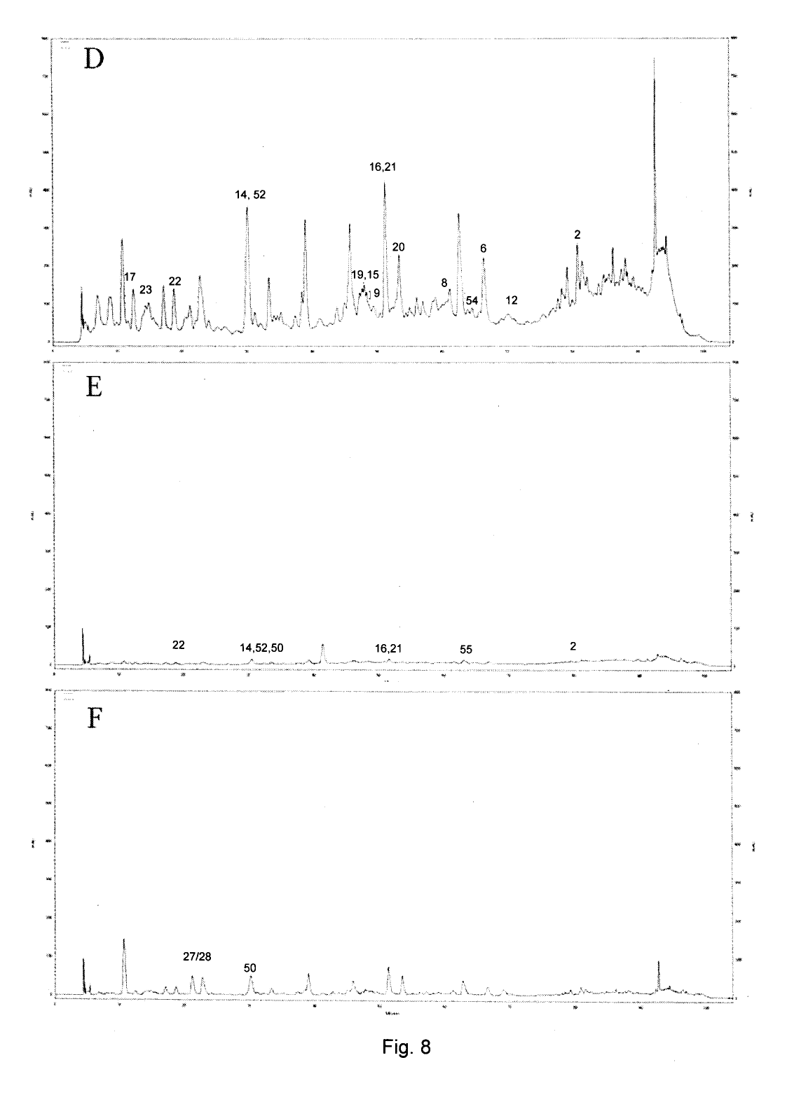

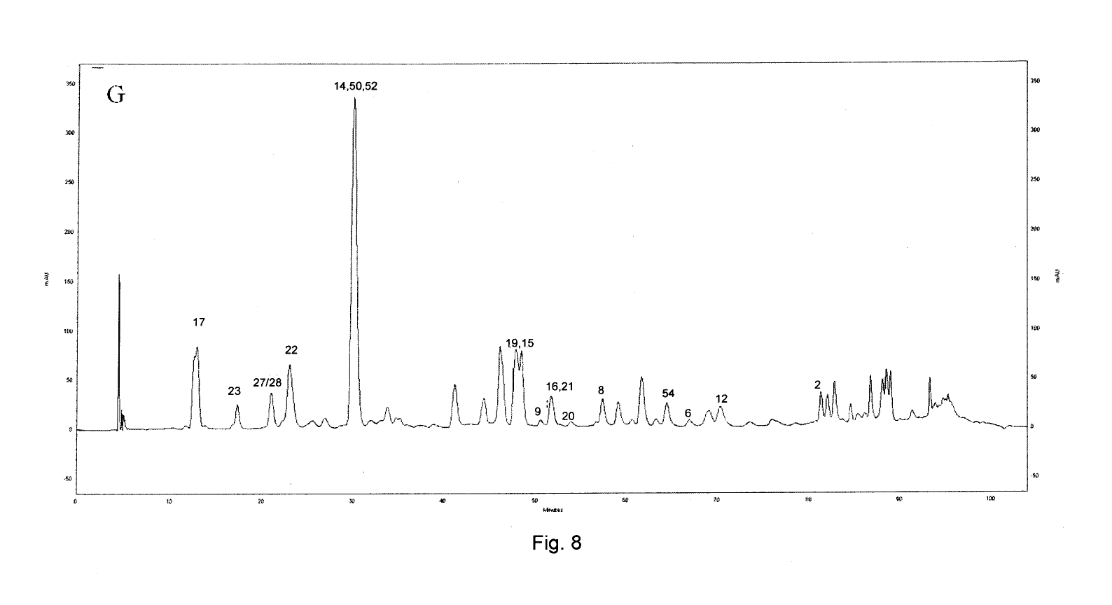

Fifty-four compounds are isolated and identified from maple syrup using a combination of nuclear magnetic resonance and mass spectral data. The maple syrup isolates are predominantly found as phenolics belonging to different sub-classes including lignans, coumarins, stilbene, and small phenolic compounds. A total of fifty-one pure phenolic compounds are selected for anticancer assays based on limited sample quantities. The identities of the pure compounds are shown in Table 12 and their presence in either the MS-EtOAc or MS-BuOH extract is based on their isolation from either extract as described above. The presence of the compounds in the MS-MeOH extract is based on HPLC analyses (chromatograms shown in FIG. 8). The relative levels of phenolic compounds in each extract are estimated by injecting samples at concentrations normalized to deliver equivalent amount of phenolics (FIG. 8).

All of the HPLC analyses are conducted as above. A Luna C18 column (250×4.6 mm i.d., 5 μM; Phenomenex), flow rate of 0.75 mL/min and injection volume of 20 μL is utilized for all of the analyses. A binary gradient solvent system consisted of solvent A (0.1% aqueous trifluoroacetic acid) and solvent B (methanol, MeOH) and is used as follows: 0-10 min, from 10 to 15% B; 10-20 min, 15% B; 20-40 min, from 15 to 30% B; 40-55 min, from 30 to 35% B; 55-65 min, 35% B; 65-85 min, from 35 to 60% B; 85-90 min, from 60 to 100% B; 90-93 min, 100% B; 93-94 min, from 100 to 10% B; 94-104 min, 10% B.

Determination of Total Phenolic Contents

The total phenolic contents of the maple syrup extracts are determined according to the Folin-Ciocalteu method and are measured as gallic acid equivalents (GAEs). Briefly, the extracts are diluted 1:100 with methanol/H₂O (1:1, v/v), and 200 μL of each sample is incubated with 3 mL of methanol/H₂O (1:1, v/v) and 200 μL of Folin-Ciocalteau reagent for 10 min at 25° C. After this, 600 μL of 20% Na₂CO₃ solution is added to each tube and vortexed. Tubes are further incubated for 20 min at 40° C. and after incubation, samples are immediately cooled in an ice bath to room temperature. Samples and standard (gallic acid) are processed identically. The absorbance is determined at 755 nm, and final results are calculated from the standard curve obtained from a Spectramax plate reader.

Cell Lines and Culture Conditions

Three human colon cancer cell lines: Caco-2 (adenocarcinoma), HT-29 (adenocarcinoma) and HCT-116 (carcinoma), and the normal colon cells, CCD-18Co, are obtained from American Type Culture Collection (Rockville, USA). Caco-2 cells are grown in EMEM medium supplemented with 10% v/v fetal bovine serum, 1% v/v nonessential amino acids, 1% v/v L-glutamine and 1% v/v antibiotic solution (Sigma). The HT-29 and HOT-116 cells are grown in McCoy's 5A medium supplemented with 10% v/v fetal bovine serum, 1% v/v nonessential amino acids, 2% v/v HEPES and 1% v/v antibiotic solution. The CCD-18Co cells are grown in EMEM medium supplemented with 10% v/v fetal bovine serum, 1% v/v nonessential amino acids, 1% v/v L-glutamine and 1% v/v antibiotic solution and are used from a PDL (population doubling level) of 26 to 35 for all experiments. Cells are maintained at 37° C. in an incubator under a 5% CO₂/95% air atmosphere at constant humidity. The pH of the culture medium is determined using pH indicator paper (pHydrion™ Brilliant, pH 5.5-9.0, Micro Essential Laboratory, NY, USA) inside the incubator. Cells are counted using a hemacytometer and are plated at 3,000-5,000 cells per well, in a 96-well format for 24 or 48 h prior to addition of the extracts or pure compounds depending on the cell line. All of the test samples are solubilized in DMSO (<0.5% in the culture medium) and are filter sterilized (0.2 μM) prior to addition to the culture media. Additional cells are set up as control wells and subjected to the same changes in medium containing the solvent control, DMSO (not exceeding 0.5%). In addition, to evaluating multiple concentrations of each sample, we also conducted time dependent experiments (conducted over 48 and 72 h) to unravel the potential mechanisms involved in cancer chemopreventive effects of the extracts and pure compounds.

Cell Proliferation and Viability Tests

All of the extracts are tested at concentrations normalized to deliver equivalent amounts, selected at 40%, of phenolics. The antiproliferative activities of the samples are evaluated in both time (48 and 72 h) and concentration dependent (1-200 μg/mL) manner. At the end of each sample treatment, trypsinised cells (2.5 g/L trypsin, 0.2 g/L EDTA) are suspended in culture medium, counted using a Neubauer haemacytometer (Bad Mergentheim, Germany) and viability measured using Trypan blue dye exclusion. Results of proliferation and viability in treated cells are expressed as percentage of those values obtained for control (0.5% DMSO) cells. All experiments are performed in triplicate.

The MTS [3-(4,5-dimethylthiazol-2-yl)-5-(3-carboxymethoxyphenyl)-2-(4-sulfenyl)-2H-tetrazolium salt] assay is carried out according to the following method. At the end of either the 48 or 72 h of treatment with serially diluted test samples, 20 μL of the MTS reagent, in combination with the electron coupling agent, phenazine methosulfate, are added to each well, and cells are incubated at 37° C. in a humidified incubator for 3 h. Absorbance is monitored at 490 nm (OD₄₉₀) using a spectrophotometer (SpectraMax M2, Molecular Devices Corp., operated by SoftmaxPro v.4.6 software, Sunnyvale, Calif., USA), to obtain the number of cells relative to control populations. The results are expressed as the concentration that inhibit growth of cells by 50% versus control cells (control medium used as negative control) to calculate the IC₅₀ values. Data are presented as the mean±S.D. of three separate experiments for each cell line. The chemotherapeutic drug, etoposide (Sigma), is used as a positive control which provided consistent IC₅₀ values of 15-25 μM (HT-29, HCT116 and Caco-2) and 40-45 μM for the CCD-18Co cells.

Flow Cytometry Analysis of Cell Cycle Arrest Cells

(2×10⁵) are collected after the corresponding experimental periods, fixed in ice-cold ethanol:PBS (70:30, v/v) for 30 min at 4° C., further resuspended in PBS with 100 μg ml⁻¹ RNAse and 40 μg ml⁻¹ propidium iodide, and then incubated at 37° C. for 30 min. The DNA content (10,000 cells) is analyzed using a FACS Calibur instrument equipped with FACStation running FACS Calibur software (BD Biosciences, San Diego, Calif., USA). The analyses of cell cycle distribution are performed in triplicate for each treatment (tested at 50 μg/mL concentrations). The coefficient of variation, according to the ModFit LT Version 2 acquisition software package (Verity Software House, Topsham, Me., USA), is always less than 5%.

Western Blot Analysis of Cyclins Expression

After 48 or 72 h of sample treatment (tested at 50 μg/mL concentrations) respectively, the cells are washed twice with PBS and lysed in cold RIPA lysis buffer (Sigma). Lysates are centrifuged at 10,000 g for 15 min at 4° C., and protein concentration is measured using Pierce BCA protein assay kit (Thermo Scientific, Ill., USA). To determine cyclins A and D1, 30 μg protein/lane are loaded. GAPDH antibody (Santa Cruz Biotech., CA, USA) is routinely assayed for monitoring total protein load. Proteins are separated by 10-12% SDS-PAGE and transferred to nitrocellulose membranes (Bio-Rad, Hercules, Calif., USA) by electroblotting. Membranes are incubated overnight at 4° C. with the primary antibodies (Santa Cruz Biotech., CA, USA) and 1 h in the dark with the secondary antibody goat anti-mouse Li-cor 926-32220 (LI-COR Biosciences, Lincoln, Nebr. USA). After that membranes are washed twice for 10 min and proteins are detected using and scan (Odyssey, LI-COR Biosciences, Lincoln, Nebr. USA). For quantification, the density of the bands is detected with scanning densitometry, using the Odyssey Infrared Imaging System v. 1.2 (LI-COR Biosciences, Lincoln, Nebr. USA). The Western blot assays are repeated at least in duplicate.

Morphological Evaluation of Apoptosis Cells

(2.5×10⁴/mL) are separately treated for 48 or 72 h and fixed with MeOH:acetic acid (70:30, v/v) and stained with 50 mg ml⁻¹ Hoechst 33242 dye at 37° C. for 20 min. Afterwards, the cells are examined under a Nikon Eclipse TE2000-E inverted microscope (Nikon, N.Y., USA). Etoposide (Sigma) 20 μM is assayed as a standard inducer of apoptosis. Morphological evaluation of apoptosis is carried out twice for each sample.

Statistical Analysis

Two-tailed unpaired student's t-test is used for statistical analysis of the data. A p value<0.05 is considered significant.

Results

Standardization of Maple Syrup Extracts to Phenolic Contents

Three different organic solvents (ethyl acetate, butanol, and methanol), are used to prepare extracts from two dark grades of maple syrup (grades C and D) yielding a total of six different extracts viz. ethyl acetate (grade C & grade D MS-EtOAc), butanol (grade C & grade D MS-BuOH) and methanol (grade C & grade D MS-MeOH). Each of the extract is individually standardized to total phenolic content based on the Folin-Ciocalteau method (see Table 11). Based on dry weight, the phenolic levels of the grades C and D MS-EtOAc extracts contained the highest phenolic contents of 34 and 30% GAEs, respectively.

TABLE 11
Total polyphenol content (as gallic acid equivalents, GAEs) of the various
maple syrup extracts estimated by the Folin-Ciocalteau method

| Source | % GAEs |
|---|---|
| Grade C MS-BuOH | 6.24 |
| Grade C MS-MeOH | 9.37 |
| Grade C MS-EtOAc | 34.10 |
| Grade D MS-BuOH | 1.04 |
| Grade D MS-MeOH | 14.92 |
| Grade D MS-EtOAc | 30.36 |

HPLC Phenolic Profiling of Maple Syrup Extracts

Table 12 shows the identities of fifty-one phenolic compounds which are previously isolated and identified from Canadian maple syrup. The HPLC chromatograms of all of the pure isolated phenolic compounds (combined into one injection), as well as the different maple syrup extracts, are shown in FIG. 8. Based on the results above and the current HPLC analyses, the presence of the isolates in the different organic solvent extracts of maple syrup are shown in Table 12. For these HPLC analyses, all of extracts are injected at concentrations normalized to deliver equivalent phenolic levels. Among the extracts, grade D MS-BuOH contained the highest relative levels of the phenolic compounds (see FIG. 8D).

TABLE 12
Presence and relative levels of pure isolated phenolic compounds in the
different maple syrup extracts*

| | | MS- | MS- | MS- |
|---|---|---|---|---|
| Compound | Name | BuOH | EtOAc | MeOH |
| 17 | Gallic acid | + | | + |
| 10 | (E)-3,3′-dimethoxy-4,4′-dihydroxy stilbene | + | | + |
| 19 | Syringic acid | + | + | + |
| 22 | C-veratroylglycol | + | + | + |
| 54 | Quebecol | + | | |
| 6 | 1-(4-hydroxy-3-methoxyphenyl)-2-[4-(3- | + | + | + |
| | hydroxypropyl)-2-methoxyphenoxy]-propane-1,3- | | | |
| | diol (guaiacylglycerol-β-O-4′-dihydroconiferyl | | | |
| | alcohol) | | | |
| 7 | 3-[(4-[(6-dexoy-α-L-mannopyranosyl)oxy]-3- | + | | + |
| | methoxyphenyl)-5-(3,4-dimethoxyphenyl)dihydro-3- | | | |
| | hydroxy-4-(hydroxymethyl)-2(3H)-furanone | | | |
| 1 | Lyoniresinol | + | + | + |
| 11 | 2-Hydroxy-3′,4′-dihydroxyacetophenone | + | | + |
| 20 | Syringenin | + | | + |
| 23 | 1,2-benzenediol (catechol) | + | | + |
| 16 | Syringaldehyde | + | | + |
| 15 | Vanillin | + | + | + |
| 5 | 1,3-propanediol, 1-(4-hydroxy-3-methoxyphenyl)-2- | + | | + |
| | [4-[(1E)-3-hydroxy-1-propenyl]-2-methoxyphenoxy]-, | | | |
| | (1R,2R) | | | |
| 3 | 2,3-dihydro-3-(hydroxymethyl)-2-(4-hydroxy-3- | + | | + |
| | methoxyphenyl)-7-methoxy-5-benzofuranpropanol | | | |
| | (dihydrodehydrodiconiferyl alcohol) | | | |
| 55 | Ferulic acid | | | + |
| 14 | Catechaldehyde | + | | + |
| 9 | Fraxetin | + | | + |
| 21 | (E)-coniferyl alcohol (coniferol) | + | | + |
| 8 | Scopoletin | + | + | + |
| 12 | 1-(2,3,4-trihydroxy-5-methylphenyl)-ethanone | + | | + |
| 56 | p-coumaric acid | | | + |
| 2 | Secoisolariciresinol | + | + | + |
| 57 | Catechin | | | + |
| 58 | Epicatechin | | | + |
| 46 | 3′,4′,5′-Trihydroxyacetophenone | | + | + |
| 49 | 4-(dimethoxymethyl)-pyrocatechol | | + | + |
| 52 | 4-acetylcatechol | | + | + |
| 41 | 2,3-dihydroxy-1-(3,4-dihydroxyphenyl)-1-propanone | | + | + |
| 44 | Dihydroconiferyl alcohol | | + | + |
| 51 | Isofraxidin | | + | + |
| 42 | 2,3-dihydroxy-1-(4-hydroxy-3,5-dimethoxyphenyl)-1- | | + | + |
| | propanone | | | |
| 50 | Tyrosol | | + | + |
| 43 | 3-hydroxy-1-(4-hydroxy-3,5- | | + | + |
| | dimethoxyphenyl)propan-1-one | | | |
| 37 | Isolariciresinol | | + | + |
| 24 | 5-(3″,4″-dimethoxyphenyl)-3-hydroxy-3-(4′-hydroxy- | | + | + |
| | 3′-methoxybenzyl)-4-hydroxymethyl-dihydrofuran-2- | | | |
| | one | | | |
| 48 | Protocatechuic acid | | + | + |
| 29 | Threo-guaiacylglycerol-β-O-4′-dihydroconiferyl | | + | + |
| | alcohol | | | |
| 45 | 4-hydroxycatechol | | + | + |
| 25 | (erythro, erythro)-1-[4-[2-hydroxy-2-(4-hydroxy-3- | | + | + |
| | methoxyphenyl)-1-(hydroxymethyl)ethoxy]-3,5- | | | |
| | dimethoxyphenyl]-1,2,3-propanetriol | | | |
| 40 | 1,2-diguaiacyl-1,3-propanediol | | + | + |
| 27 | (threo, erythro) 1-[4-[(1R,2R)-2-hydroxy-2-(4- | | + | + |
| | hydroxy-3-methoxyphenyl)-1- | | | |
| | (hydroxymethyl)ethoxy]-3-methoxyphenyl)-1,2,3- | | | |
| | propanetriol | | | |
| 28 | (threo, threo) 1-[4-[(1R,2R)-2-hydroxy-2-(4-hydroxy- | | + | + |
| | 3-methoxyphenyl)-1-(hydroxymethyl)ethoxy]-3- | | | |
| | methoxyphenyl]-1,2,3-propanetriol | | | |
| 33 | Leptolepisol D | | + | + |
| 39 | Sakuraresinol | | + | + |
| 26 | (erythro, threo)-1-[4-[2-hydroxy-2-(4-hydroxy-3- | | + | + |
| | methoxyphenyl)-1-(hydroxymethyl)ethoxy]-3,5- | | | |
| | dimethoxyphenyl]-1,2,3-propanetriol | | | |
| 38 | Icariside E4 | | + | + |
| 36 | Syringaresinol | | + | + |
| 32 | Acernikol | | + | + |
| 35 | (1S,2R)-2-[2,6-dimethoxy-4-[(1S,3aR,4S,6aR)- | | + | + |
| | tetrahydro-4-(4-hydroxy-3,5-dimethoxyphenyl)- | | | |
| | 1H,3H-furo[3,4-c]furan-1-yl]phenoxy]-1-(4-hydroxy- | | | |
| | 3-methoxyphenyl)-1,3-propanediol | | | |
| 31 | 2-[4-[(2S,3R)-2,3-dihydro-3-(hydroxymethyl)-5-(3- | | + | + |
| | hydroxy propyl)-7-methoxy-2-benzofuranyl]-2,6- | | | |
| | dimethoxyphenoxy]-1-(4-hydroxy-3- | | | |
| | methoxyphenyl)-1,3-propanediol | | | |
| 34 | Buddenol E | | + | + |
| 3 | Dehydroconiferyl alcohol | + | | |
| 13 | 2,4,5-trihydroxyacetophenone | + | | |
| 18 | trimethyl gallic acid methyl ester | + | | |
| 30 | erythro-1-(4-hydroxy-3-methoxyphenyl)-2-[4-(3- | | + | |
| | hydroxypropyl)-2,6-dimethoxyphenoxy]-1,3- | | | |
| | propanediol | | | |
| 47 | 3,4-dihydroxy-2-methylbenzaldehyde | | + | |
| 53 | phaseic acid | | + | |

The presence of the compounds in MS-BuOH and MS-EtOAc extracts are determined by their previous phytochemical isolation from these extracts as described above while the presence of compounds in MS-MeOH was determined by HPLC analyses.

Antiproliferative Activities of Maple Syrup Extracts on Colon Cells

To correlate the antiproliferative efficacy of the maple syrup extracts to their phenolic contents, the samples are individually normalized to deliver equivalent phenolic content in the bioassays. Initially, the maple syrup extracts are individually evaluated for effects on cell viability and in all cases, cell viability exceeded 90% suggesting that the extracts are not cytotoxic (data not shown). All of the maple syrup extracts inhibited proliferation of the colon cancer (HCT-116, Caco-2 and HT-29) cell lines in a time and concentration dependent manner (Table 13). The antiproliferative results indicated clear differences between the two grades of maple syrup where grade D is more active than grade C (˜3-fold in MS-BuOH extract, and ˜1.5-fold in the MS-MeOH and MS-EtOAc extracts).

TABLE 13
Antiproliferative activty of maple syrup extracts against human
colon cell lines after 48 or 72 h treatment.^{a}

| Maple Syrup | HCT-116 | | HT-29 | |
|---|---|---|---|---|
| Extract | 48 h | 72 h | 48 h | 72 h |
| Grade C MS-BuOH | 60.7 ± 5.8 | 49.2 ± 1.6 | 87.2 ± 2.1 | 64.5 ± 4.6 |
| Grade C MS-MeOH | 133.7 ± 3.2 | 77.3 ± 2.7 | 150.8 ± 2.2 | 82.0 ± 2.1 |
| Grade C MS-EtOAc | 254.0 ± 7.4 | 152.4 ± 6.4 | 284.8 ± 5.6 | 171.6 ± 4.3 |
| Grade D MS-BuOH | 20.1 ± 1.9 | 11.2 ± 2.1 | 25.5 ± 2.0 | 14.8 ± 3.0 |
| Grade D MS-MeOH | 106.8 ± 4.6 | 78.8 ± 1.6 | 122.1 ± 2.8 | 80.5 ± 2.3 |
| Grade D MS-EtOAc | 148.1 ± 5.3 | 122.3 ± 5.8 | 173.8 ± 6.6 | 141.0 ± 5.6 |
| Maple Syrup | Caco-2 | | CCD-18Co | |
| Extract | 48 h | 72 h | 48 h | 72 h |
| Grade C MS-BuOH | 89.8 ± 3.1 | 65.6 ± 3.6 | 176.9 ± 6.1 | 128.0 ± 3.5 |
| Grade C MS-MeOH | 131.1 ± 2.9 | 86.9 ± 3.2 | 247.7 ± 4.3 | 179.4 ± 7.2 |
| Grade C MS-EtOAc | 267.0 ± 5.5 | 166.1 ± 3.1 | n.d. | 238.2 ± 8.1 |
| Grade D MS-BuOH | 24.7 ± 2.6 | 14.7 ± 1.2 | 67.8 ± 2.5 | 54.3 ± 3.6 |
| Grade D MS-MeOH | 112.6 ± 4.1 | 76.3 ± 1.7 | 208.8 ± 2.4 | 153.7 ± 1.7 |
| Grade D MS-EtOAc | 171.8 ± 4.5 | 142.1 ± 5.1 | n.d. | 230.8 ± 6.9 |

^{a}IC₅₀ (μg/mL) is defined as the concentration required to achieve 50% inhibition over control cells (DMSO 0.5%); IC₅₀ values are shown as mean ± S.D. from three independent experiments. n.d. not detected

Overall, among the different colon cancer cell lines, the HCT-116 cells are the most sensitive to the maple syrup extract treatments compared to the Caco-2 and HT-29 cells. The most potent antiproliferative effects against the colon cancer cell lines are observed with the MS-BuOH extracts from grades C and D with IC₅₀ values ranging from 20-89 μg/mL at 48 h and 11-65 μg/mL at 72 h, respectively. The IC₅₀ values after treatment with the MS-MeOH extracts from grades C and D ranged from 112-50 μg/mL at 48 h and from 78-86 μg/mL at 72 h, respectively. Finally, moderate activity is observed with the MS-EtOAc extracts from grades C and D with IC₅₀ values ranging from 148-284 μg/mL at 48 h, and 122-171 μg/mL at 72 h, respectively (Table 13). Notably, there are significant differences between the IC₅₀ values observed with the extracts against the colon cancer cells compared to the normal colon (CCD-18Co) cells with over 1.5, 2 and 2.5 fold for MS-BuOH, MS-MeOH, and MS-EtOAc extracts, respectively (Table 13).

Effects of Maple Syrup Extracts On Cell Cycle Distribution Analysis and Cyclins Expression

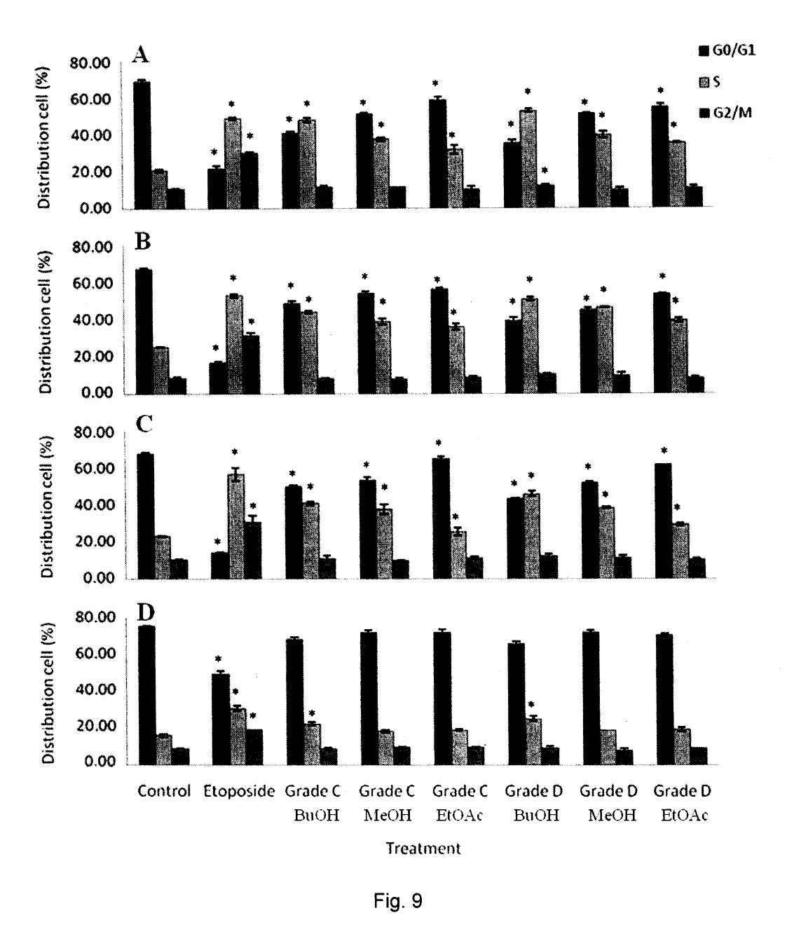

Inhibition of cell proliferation is further examined by measuring the cell cycle distribution after treatment with each maple syrup extract (at 50 μg/mL test concentrations). After 48 h, the HCT-116, Caco-2, and HT-29 control cells (i.e. without sample treatments) are distributed as follows: 53.6-59.0% in G₀/G₁ phase, 30.2-36.9% in S phase and 9.5-11.1% in G₂/M phase (data not shown). After the further time point of 72 h, the proportion of the control cells in the G₀/G₁ phase increased to 66.58-68.48% whereas the cells in S and G₂/M phases decreased to 20.7-25.2% and to 8.4-10.7%, respectively (FIGS. 9A-C).

After 48 h, all of the extracts, except MS-MeOH, showed significant increase of cells in S phase (p<0.05) concomitant with a decrease in G₀/G₁ (p<0.05) and a slight increase in the G₂/M phase (results not shown). Consistent with the antiproliferative activity, the MS-BuOH extract showed the most pronounced changes in cell cycle distribution. Specifically, MS-BuOH showed clear arrest of the cells in the S-phase ranging from 41.8-52.0% (p<0.05) on all cell lines, while that of the MS-MeOH and MS-EtOAc extracts showed ranges of 34.8-47.1% (p<0.05) and 32.6-40.61% (p<0.05), respectively.

After 72 h, the cell cycle arrests are maintained significantly by all of the extracts, including MS-MeOH, against the colon cancer cell lines (FIG. 2). The MS-BuOH exhibited ˜1.8-fold and ˜2.0-fold increases when compared to control cells in the S phase which is accompanied by a decrease of cells in G₀/G₁ phase (p<0.05) for grades C and D, respectively. In addition, significant increase (p<0.05) of the G₂/M ratio is also found. A similar trend is observed in the colon cancer cell lines treated with MS-MeOH and MS-EtOAc extracts with a 1.5-fold and 1.3-fold increase in the S phase of cell cycle arrest from grade C, and ˜1.4-fold and ˜1.2-fold from grade D, respectively.

Among the colon cancer cell lines, similar to the trend in antiproliferative effects, the HCT-116 cells are most sensitive to cell cycle distribution after the treatments. Moreover, incubation of CCD-18Co cells with the maple syrup extracts for 48 and 72 h did not cause significant changes in cell cycle when compared with control cells. However, slight but significant changes in the S phase is observed with the incubation of the MS-BuOH extracts (from both grades) at 72 h, and with incubation of etoposide (at 50 μM; used as a positive control (see FIG. 9D).

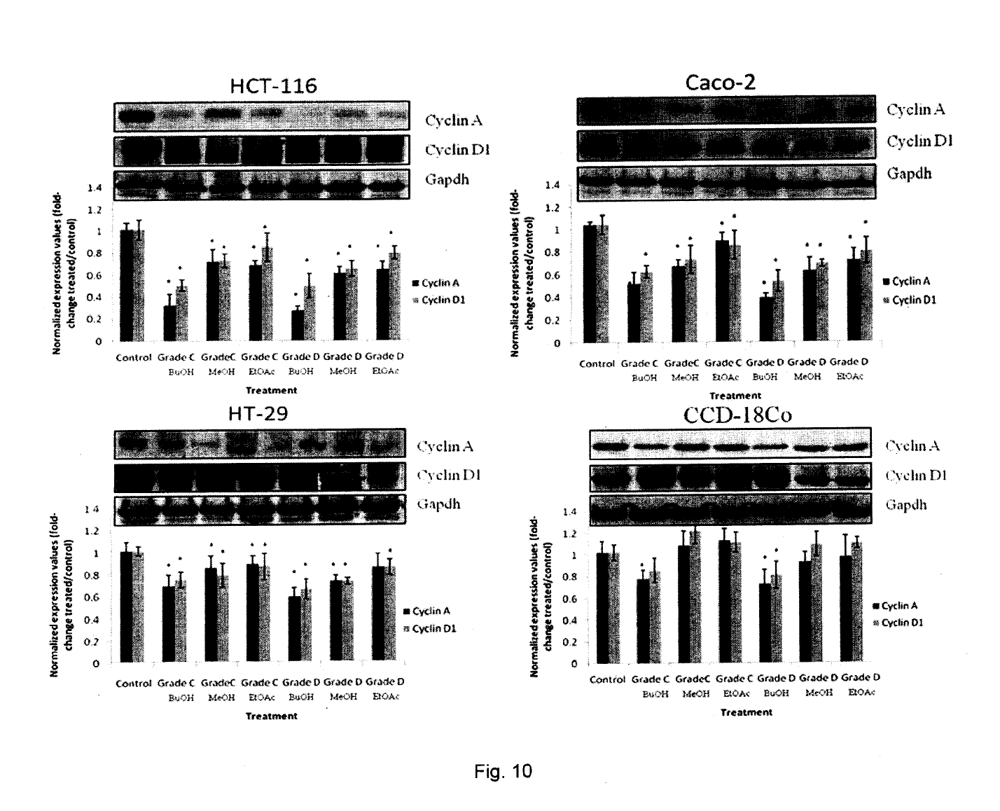

To gain further insights into the molecular mechanisms of anticancer action, the maple syrup extracts are evaluated for effects on the expression of cyclins A and D, proteins integral in cell cycling that are up-regulated in the S phase in normal cells. All the extracts significantly decreased the expression of cyclin D1 and A at 48 h (data not shown) and 72 h (see FIG. 10). These results indicated that the phytochemicals present in maple syrup extracts can inhibit the proliferation of colon cancer cells by blocking the progression of cell cycle at S-phase due to decrease of expression of cyclin D1 and A.

Effects of Maple Syrup Extracts on Apoptosis of Colon Cells

Apart from cell cycle arrest, another possible mechanism that would be related to the antiproliferative activity of the maple syrup extracts could be through the induction of apoptosis (programmed cell death). Therefore, morphological evaluation of apoptosis is conducted by monitoring for changes in nuclear chromatin distribution stained by the DNA-binding fluorochrome, Hoechst 33242 dye. Incubation of the colon cancer and normal cells with the extracts mirrored the pattern followed by untreated cells, thus indicating the absence of apoptosis.

Antiproliferative Activities of Isolated Phenolics from Maple Syrup on Colon Cells

The antiproliferative activities of phenolics previously isolated from maple syrup extracts are evaluated after both 48 and 72 h of treatment (Table 14). All of the pure compounds inhibited proliferation of the HCT-116, Caco-2 and HT-29 colon cancer cell lines and are more effective against these cells compared to the normal CCD-18Co colon cells (over 1.5 fold). Similar to the observation of the antiproliferative effects of the extracts, the HCT-116 cells are the most sensitive among the cell lines to the purified compounds.

TABLE 14
Antiproliferative activity of pure isolated compounds from maple syrup S
extracts against human colon cell lines after 48 and 72 h treatment

| | HCT-116 | | Caco-2 | | HT-29 | | CCD-8Co | |
|---|---|---|---|---|---|---|---|---|
| Compound | 48 h | 72 h | 48 h | 72 h | 48 h | 72 h | 48 h | 72 h |
| 17 | 64.9 ± 2.3 | 42.1 ± 1.9 | n.d. | 89.3 ± 1.7 | n.d. | n.d. | n.d. | n.d. |
| 19 | 119.0 ± 1.3 | 108.0 ± 0.9 | n.d. | n.d. | 126.0 ± 1.8 | 116.3 ± 1.8 | n.d. | n.d. |
| 22 | 82.9 ± 2.1 | 66.7 ± 1.5 | 100.8 ± 1.6 | 90.8 ± 1.0 | 96.0 ± 1.5 | 88.6 ± 1.3 | n.d. | n.d. |
| 54 | 95.3 ± 0.6 | 76.2 ± 1.2 | 98.2 ± 1.5 | 78.5 ± 1.4 | 103.2 ± 1.7 | 86.1 ± 0.8 | n.d. | 120.4 ± 1.5 |
| 6 | 107.3 ± 2.3 | 98.6 ± 3.2 | 93.9 ± 2.0 | 88.5 ± 1.0 | n.d. | 110.3 ± 2.0 | n.d. | n.d. |
| 1 | n.d. | n.d. | n.d. | n.d. | n.d. | n.d. | n.d. | n.d. |
| 20 | 123.5 ± 1.1 | 108.0 ± 1.5 | n.d. | n.d. | 126.4 ± 3.0 | 112.9 ± 1.5 | n.d. | n.d. |
| 23 | 86.0 ± 2.4 | 63.0 ± 1.3 | 71.5 ± 2.3 | 64.1 ± 2.5 | 99.4 ± 2.1 | 70.3 ± 2.0 | n.d. | 115.0 ± 2.7 |
| 16 | 66.8 ± 1.3 | 56.3 ± 1.8 | 59.4 ± 2.7 | 35.9 ± 2.4 | 90.7 ± 3.0 | 68.6 ± 1.4 | n.d. | n.d. |
| 15 | 120.3 ± 1.0 | 109.9 ± 1.7 | n.d. | n.d. | n.d. | 113.7 ± 1.5 | n.d. | n.d. |
| 55 | 119.1 ± 1.4 | 104.5 ± 1.7 | n.d | n.d. | 128.2 ± 1.1 | 120.7 ± 1.3 | n.d. | n.d. |
| 14 | 63.3 ± 1.3 | 52.9 ± 1.4 | 73.2 ± 1.3 | 58.9 ± 1.9 | 71.6 ± 1.5 | 63.9 ± 1.6 | n.d. | 126.2 ± 4.6 |
| 9 | 112.0 ± 1.8 | 84.5 ± 1.6 | n.d. | 101.2 ± 1.3 | 101.1 ± 1.2 | 93.4 ± 2.6 | n.d. | n.d. |
| 21 | 111.4 ± 1.5 | 84.6 ± 1.9 | 111.5 ± 2.0 | 96.4 ± 2.9 | n.d. | 113.2 ± 1.9 | n.d. | n.d. |
| 8 | 68.1 ± 1.0 | 60.6 ± 2.0 | 89.3 ± 0.9 | 82.0 ± 2.0 | 78.7 ± 1.2 | 70.0 ± 1.3 | n.d. | 102.8 ± 5.9 |
| 12 | 95.8 ± 1.6 | 75.0 ± 2.7 | 102.7 ± 2.7 | 90.2 ± 1.9 | 94.6 ± 2.5 | 84.4 ± 2.0 | n.d. | 147.7 ± 6.1 |
| 2 | 78.0 ± 2.3 | 59.1 ± 1.5 | 85.0 ± 3.8 | 70.9 ± 2.3 | 88.7 ± 2.2 | 68.2 ± 1.4 | n.d. | 99.6 ± 4.9 |
| 52 | 95.0 ± 3.4 | 57.7 ± 3.4 | 89.2 ± 1.9 | 78.6 ± 1.2 | 109.8 ± 2.3 | 100.3 ± 1.5 | n.d. | n.d. |
| 50 | 115.4 ± 1.5 | 94.3 ± 1.8 | 112.1 ± 1.9 | 100.2 ± 2.6 | 128.0 ± 2.3 | 114.2 ± 1.8 | n.d. | 144.7 ± 4.1 |
| 27/28 | 102.2 ± 2.3 | 79.8 ± 2.1 | 103.3 ± 2.0 | 85.2 ± 1.2 | 110.3 ± 2.6 | 92.3 ± 1.8 | n.d. | 132.3 ± 2.7 |

Overall, the compounds are ranked in order of highest, moderate, and lowest antiproliferative activities based on their IC₅₀ values against HCT-116 cells at 72 h. Thus, the highest antiproliferative effects against the colon cancer cells (IC₅₀=42-67 μM) are observed for compounds 14, 17, 16, 52, 2, 8, 23 and 22. Moderately active compounds (IC₅₀=75-85) are 12, 54, (27 or 28), 9 and 21 and lowest active compounds are 50, 6, 55, 20, 19 and 15 (IC₅₀=94-110 μM) (Table 14). Notably, the most active compounds are also present in higher relative levels in the MS-BuOH extract (see FIG. 8D), which could account for its superior effects compared to the other extracts.

According to one embodiment of the present invention, the anticancer effects of phenolic-enriched extracts of two dark grades of maple syrup and fifty-one of their purified phenolic isolates on a panel of human colon cancer and normal colon cells are investigated. According to another embodiment, the underlying molecular mechanisms of anticancer action of the maple syrup extracts is also investigated.

After normalization to phenolic content, the results demonstrated that the most potent extract is MS-BuOH followed by the MS-MeOH and MS-EtOAc extracts. In addition, the antiproliferative effects observed with the extracts are more pronounced on colon cancer cells compared to the normal cells. Similar to our observations, plant extracts have been indicated to show selective growth inhibitory activity against different human colon cancer cells with less effect on normal cell lines. The selectivity of the extracts to colon tumorigenic compared to non-tumorigenic colon cells suggests that they may have potential as chemopreventive agents.

Differences in effects between two dark grades of maple syrup are apparent. Overall, when normalized to phenolic content, the grade D maple syrup extracts are more active than the grade C extracts which could probably be due to higher concentration and/or synergistic combination of the most active phenolics. In fact, the relative levels of the most active isolates are higher in the grade D MS-BuOH extract.

The antiproliferative activities exhibited by the extracts are not due to cytotoxicity since the viability of the treatment cells is not significantly different from that of control cells. To further investigate the mechanism of antiproliferative effects of the maple syrup extracts on the colon cancer cells, the induction of apoptosis is determined. Notably, none of the extracts induced the chromatin condensation on either the cancer or normal cells, confirming the absence of the apoptosis. However, all of the maple syrup extracts significantly arrested cell cycle in the S-phase of all of the colon cancer cells in a time dependent manner. Similar to the observations in the antiproliferative assays, the MS-BuOH extracts of both grades induced greater arrest in the S-phases and slight but not significant increases, in the G₂/M phases for all of the colon cancer cell lines, except the HCT-116 colon cells at 72 h (FIG. 9A). On the other hand, there are no significant changes in the cell cycles of the normal colon cells after treatment with the extracts with the exception of slight but statistically significant cell increase at the S phase after the treatment with the MS-BuOH extracts (FIG. 9D). Similar to our observations, phenolic-enriched extracts and their purified isolates have also been shown to induce S-phase arrest in cancer cells in vitro.

Cell cycle progression is regulated by the activity of cyclins, a family of proteins which activate the so-called cyclin-dependent-kinases (Cdks). Abnormalities of several cyclins in particular, cyclin A, E and D, have been reported in cancer cells. Our results showed that extract treatments decreased the levels of cyclin A and D1 at the same way observed in cell cycle analysis. Cyclins A and D1 are detectable in the S phase and increase during cell cycle progression to G₂/M phase. Therefore, a decrease in cyclin D1 expression is correlated with S-phase arrest since the cycle cannot progress to G₂ phase. Thus, the low expression of these cyclins after the extract treatments could be explained, in part, by the prevention of the cells transitioning to the G₂/M phase.

The antiproliferative activities of fifty-four isolated phenolic compounds from the maple syrup extracts is determined to evaluate which constituent could be involved in this activity. The results indicated that several compounds (in particular, gallic acid, catechaldehyde, syringaldehyde, 4-acetylcathecol, secoisolariciresinol and scopoletin) inhibited growth of the cancer cell lines at concentrations ranging from 42 to 60 μM. The relative higher levels of several of these most active compounds in the MS-BuOH extracts (see FIG. 8D), could explain its higher observed anticancer potential compared to the other extracts. Also, it is possible that multiple compounds present in this extract could exhibit additive, complementary and/or synergistic effects which could potentiate its bioactivity.

In conclusion, the results indicated that maple syrup phenolic enriched extracts, does not induce apoptosis but inhibits the growth of colon cancer cells due to cell cycle arrest in the S-phase which is associated with a concomitant decrease in cyclins A and D1 levels. The antiproliferative effects observed by the maple syrup extracts are more pronounced on the human colon cancer than normal colon cells in both time and concentration dependent manners. The superior activity of the MS-BuOH extract compared to the other extracts could probably be due to the presence of the most active phenolic compounds such as gallic acid, catechaldehyde, syringaldehyde and/or scopoletin.

### Example 4

### Effects of Maple (Acer) Plant Part Extracts on Proliferation, Apoptosis, and Cell Cycle Arrest of Human Tumorigenic and Non-Tumorigenic Colon Cells

Cell Lines and Culture Conditions.

The extracts are solubilized in DMSO and normalized based on their phenolic content to evaluate their antiproliferative activities against the colon cell lines. Human colon cancer cell lines, Caco-2 (adenocarcinoma), HT-29 (adenocarcinoma) and HCT-116 (carcinoma), and the normal colon cells, CCD-18Co, are obtained from American Type Culture Collection (ATCC, Rockville, USA). The Caco-2 cells are grown in EMEM medium supplemented with 10% v/v fetal bovine serum, 1% v/v nonessential amino acids, 1% v/v L-glutamine and 1% v/v antibiotic solution (Sigma). The HT-29 and HCT-116 cells are grown in McCoy's 5A medium supplemented with 10% v/v fetal bovine serum, 1% v/v nonessential amino acids, 2% v/v HEPES and 1% v/v antibiotic solution, The CCD-18Co cells are grown in EMEM medium supplemented with 10% v/v fetal bovine serum, 1% v/v nonessential amino acids, 1% v/v L-glutamine and 1% v/v antibiotic solution and are used from passage between 26 to 35 for all experiments. Cells are maintained at 37° C. in an incubator under a 5% CO₂/95% air atmosphere at constant humidity. The pH of the culture medium is determined using pH indicator paper (pHydrion™ Brilliant, pH 5.5-9.0, Micro Essential Laboratory, NY, USA) inside the incubator. Cells are counted using a hemacytometer and are plated at 3,000-5,000 cells per well, in a 96-well format for 24 or 48 h prior to sample treatment depending on the cell line. All of the test samples are solubilized in DMSO (<0.5% in the culture medium) by sonication and are filter sterilised (0.2 μm) prior to addition to the culture media. Control cells are also run in parallel and subjected to the same changes in medium with 0.5% DMSO.

Cell Proliferation and Viability Tests (Trypan Blue Exclusion and MTS Assays).

At the end of either 48 or 72 h of sample treatment, trypsinised cells (2.5 g/L trypsin, 0.2 g/L EDTA) are suspended in cell culture medium, counted using a Neubauer haemacytometer (Bad Mergentheim, Germany) and viability measured using Trypan blue dye exclusion. Results of proliferation and viability in extract-treated cells are expressed as percentage of those values obtained compared to control (0.5% DMSO) cells. All experiments are performed in triplicate.

The MTS assay is carried out as described above. At the end of 48 or 72 h of treatment with serially diluted test samples, 20 μL of the MTS reagent, in combination with the electron coupling agent, phenazine methosulfate, is added to the wells and cells are incubated at 37° C. in a humidified incubator for 3 h. Absorbance at 490 nm (OD₄₉₀) is monitored with a spectrophotometer (SpectraMax M2, Molecular Devices Corp., operated by SoftmaxPro v.4.6 software, Sunnyvale, Calif., USA), to obtain the number of cells relative to control populations. In addition, 20 μL of a standard of the chemotherapeutic drug, etoposide (4 mg/mL), is also assayed to evaluate its effects on cell proliferation. The final results are expressed as the concentration that inhibit growth of cell by 50% vs. control cells (control medium used as negative control) i.e. the IC₅₀ value. Data are presented as the mean±S.D. of three separate experiments on each cell line. The chemotherapeutic drug, etoposide, is used as a positive control and provided consistent IC₅₀ values of 10-20 μM (HT29, HCT116 and Caco-2) and 30-40 μM for the CCD-18Co cells.

Flow Cytometry Analysis of Cell Cycle.

Cells (2×10⁵) are collected after the corresponding experimental periods, fixed in ice-cold ethanol:PBS (70:30, v/v) for 30 min at 4° C., further resuspended in PBS with 100 μg/mL RNAse and 40 μg/mL propidium iodide, and incubated at 37° C. for 30 min. DNA content (10,000 cells) is analysed using a FACS Calibur instrument equipped with FACStation running FACS Calibur software (BD Biosciences, San Diego, Calif., USA). The analyses of cell cycle distribution are performed in triplicate for each treatment. The coefficient of variation, according to the ModFit LT Version 2 acquisition software package (Verity Software House, Topsham, Me., USA), is always less than 5%.

Morphological Evaluation of Apoptosis.

Cells (2.5×10⁴/mL) are treated for 48 and 72 h and fixed with methanol: acetic acid (3:1, v/v) and stained with 50 mg/mL Hoechst 33242 dye at 37° C. for 20 min. Afterwards, the cells are examined under a Nikon Eclipse TE2000-E inverted microscope (Nikon, N.Y., USA). Etoposide (Sigma) 20 μM is assayed as a standard inducer of apoptosis. Morphological evaluation of apoptosis is carried twice for each sample.

Statistical Analysis.

Two-tailed unpaired student's t-test is used for statistical analysis of the data. A p value<0.05 is considered significant.

Standardization of Maple Plant Part Extracts.

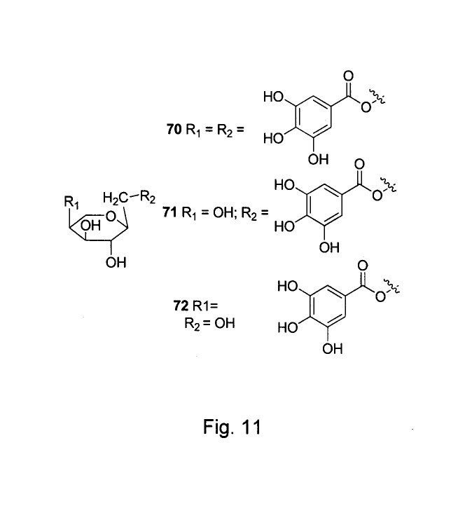

Various plant parts of two maple species are subjected to extraction protocols to enrich them in phenolic contents. The total phenolic content of all of the extracts are evaluated by the Folin-Ciocalteu method and is measured as gallic acid equivalents (GAEs) which ranged from 28.65-63.73 mg/L (Table 15). The extracts are further standardized to ginnalin-A (70), ginnalin-B (71) and ginnalin-C (72) contents (chemical structures shown in FIG. 11), which are phenolic compounds that are present in Acer (maple) species.

TABLE 15
Total phenolic content of maple plant part extracts estimated by the Folin-
Ciocalteau method in 125 mg/L of each sample.

| Source | mg/L | % |
|---|---|---|
| Red maple leaves | 56.63 | 45.30 |
| Red maple stems | 63.73 | 50.98 |
| Red maple barks | 40.30 | 32.24 |
| Red maple sapwoods | 32.40 | 25.92 |
| Sugar maple leaves | 43.79 | 35.04 |
| Sugar maple stems | 54.57 | 43.65 |
| Sugar maple barks | 41.06 | 32.85 |
| Sugar maple sapwoods | 32.24 | 25.79 |

The HPLC chromatograms of the extracts from the different plant parts of the Red maple and Sugar maple are shown in FIGS. 12A and 12B, respectively. In the HPLC chromatograms, peaks 1, 2, and 3 correspond to ginnalins-A, B, and C, respectively. Due to the similarity in chemical structures of ginnalins-B and C (FIG. 11), it is not surprising to observe that peaks 2 and 3 co-eluted in the HPLC chromatogram (FIG. 12A). Among these three phenolics, ginnalin-A is the predominant constituent present in the maple extracts. Also, among the extracts, the leaf extract from the Red maple species contained the highest level of ginnalin-A of 45% by weight. On the contrary, the leaf extract of the Sugar maple species contained lower quantities of ginnalin A, estimated from the standard curve to be <3% by weight. The twigs/stem of the Red maple tree contained the second highest level of ginnalin-A of 24.9% by weight.

Antiproliferative Activity on Cancer Colon Cells by Extracts.

The extracts are normalized to deliver equivalent amount of phenolics (50% dry weight) in the antiproliferative assays. All of the maple extracts inhibited the proliferation of the colon cancer (HCT-116, Caco-2 and HT-29) cell lines in both time-dependent and concentration-dependent manner (Table 16). Among the colon cancer cells, the HCT-116 cells are most sensitive to all of the maple extract treatments compared to the Caco-2 and HT-29 cell lines (Table 16). There is a significant difference between the IC₅₀ values of the extracts against the colon cancer cells compared to the CCD-18Co normal cells (over 2-fold).

TABLE 16
Antiproliferative effects of various maple plant part extracts against
human colon cell lines after 48 and 72 h treatment

| | HCT-116 | | HT-29 | |
|---|---|---|---|---|
| | 48 h | 72 h | 48 h | 72 h |
| Source | IC50^{a} | IC50^{a} | IC50^{a} | IC50^{a} |
| Sugar maple | 46.7 ± 4.1 | 35.3 ± 3.0 | 144.1 ± 5.3 | 91.6 ± 8.5 |
| leaves | | | | |
| Red maple leaves | 97.4 ± 3.6 | 87.6 ± 3.7 | 166.0 ± 8.7 | 134.0 ± 12.1 |
| Sugar maple | 75.6 ± 5.1 | 55.7 ± 4.0 | 163.3 ± 8.1 | 111.1 ± 2.0 |
| stems | | | | |
| Red maple stems | 159.3 ± 11.8 | 101.6 ± 8.9 | 183.8 ± 7.2 | 146.3 ± 9.0 |
| Sugar maple | 89.0 ± 4.9 | 52.2 ± 3.9 | 125.3 ± 6.0 | 91.7 ± 8.2 |
| barks | | | | |
| Red maple barks | 82.4 ± 1.7 | 59.8 ± 1.9 | 104.6 ± 2.0 | 92.5 ± 2.3 |
| Sugar maple | 226.3 ± 7.9 | 165.3 ± 4.3 | 249.0 ± 6.5 | 178.3 ± 9.4 |
| sapwoods | | | | |
| Red maple | 173.5 ± 3.9 | 147.9 ± 3.0 | 188.9 ± 2.8 | 154.9 ± 2.5 |
| sapwoods | | | | |
| | Caco-2 | | CCD-18Co | |
| | 48 h | 72 h | 48 h | 72 h |
| Source | IC50^{a} | IC50^{a} | IC50^{a} | IC50^{a} |
| Sugar maple | 127.0 ± 9.6 | 80.7 ± 4.7 | 305.7 ± 7.3 | 190.3 ± 9.3 |
| leaves | | | | |
| Red maple leaves | 149.1 ± 9.8 | 98.0 ± 5.1 | n.d. | 251.9 ± 6.3 |
| Sugar maple | 149.3 ± 8.6 | 101.2 ± 7.1 | 334.8 ± 9.8 | 220.6 ± 8.8 |
| stems | | | | |
| Red maple stems | 170.2 ± 5.8 | 145.5 ± 7.4 | n.d. | 347.9 ± 10.5 |
| Sugar maple | 100.6 ± 5.8 | 77.5 ± 3.8 | 235.8 ± 7.0 | 188.0 ± 5.2 |
| barks | | | | |
| Red maple barks | 101.4 ± 2.6 | 85.8 ± 2.6 | n.d. | 191.1 ± 5.0 |
| Sugar maple | 243.0 ± 7.1 | 179.6 ± 4.9 | n.d. | 287.6 ± 8.5 |
| sapwoods | | | | |
| Red maple | 169.7 ± 4.3 | 137.2 ± 3.4 | 357.8 ± 7.0 | 252.9 ± 5.7 |
| sapwoods | | | | |

^{a}IC₅₀ (in μg/mL) is defined as the concentration required to achieve 50% inhibition over control cells (DMSO 0.5%); IC₅₀ values are shown as mean ± S.D. from three independent experiments; n.d. = not detected. The chemotherapeutic agent, etoposide, provided consistent IC₅₀ values of 10-20 μM (HT29, HCT116 and Caco-2) and 30-40 μM for the CCD-18Co cells.

After 72 h, the highest antiproliferative effects against the colon cancer cell lines are observed from the leaves and stem extracts of the Red maple species with IC₅₀ values ranging from 35-91 mg/mL and from 55-111 μg/mL, respectively. On the other hand, the IC₅₀ values after treatment with the bark extracts from the Red and Sugar maple species ranged from 52-91 and from 59-92 μg/mL, respectively. Moderate activity is found in the leaves and stem extracts from the Sugar maple species (IC₅₀=87-134 and 101-146 μg/mL, respectively). Finally, extracts from heartwood of both species of maple tree showed IC₅₀ values ranging from 127-183 μg/mL) (Table 16).

Overall, among the extracts, the leaves and stem extracts showed greater effects than the bark and sapwood extracts. Also, between the two maple species, extracts of the Red maple showed greater antiproliferative activity than from the Sugar maple. In all cases, cell viability is always above 90% at tested doses so the extracts are not cytotoxic (data not shown). Notably, plant-derived extracts have been reported to show selective growth inhibitory activity against human colon cancer cells compared to normal cell lines.

Antiproliferative Activity on Cancer Colon Cells by Ginnalins.

Because ginnalins are the major constituents in the leaf extract of the Red maple species, we evaluated whether these compounds are contributing towards the antiproliferative effects by the MTS assay. Table 17 shows the antiproliferative activities of ginnalins-A, B and C on the colon cancer and normal colon cells. Among the three pure phenolic compounds, ginnalin-A showed the best activity with IC₅₀ values ranging from 16-24 μg/mL. Also, among the cell lines, the HCT-116 colon cancer cells are most sensitive to this compound. All ginnalins showed selective activity towards the colon cancer cells than the normal colon cells similar to the observation with the maple plant part extracts.

TABLE 17
Antiproliferative effects of ginnalins A, B and C against human
colon cell lines after 48 and 72 h treatment

| | HCT-116 | | HT-29 | |
|---|---|---|---|---|
| | 48 h | 72 h | 48 h | 72 h |
| Compounds | IC₅₀^{a} | IC₅₀^{a} | IC₅₀^{a} | IC₅₀^{a} |
| Ginnalin A | 21.5 ± 1.6 | 16.3 ± 2.1 | 31.0 ± 2.6 | 24.1 ± 1.3 |
| Ginnalin B | 25.1 ± 1.8 | 20.0 ± 2.0 | 36.2 ± 1.5 | 27.3 ± 0.6 |
| Ginnalin C | 27.0 ± 1.9 | 22.3 ± 2.4 | 33.8 ± 2.0 | 30.1 ± 1.3 |
| | Caco-2 | | CCD-18Co | |
| | 48 h | 72 h | 48 h | 72 h |
| Compounds | IC₅₀^{a} | IC₅₀^{a} | IC₅₀^{a} | IC₅₀^{a} |
| Ginnalin A | 28.8 ± 1.8 | 21.7 ± 1.0 | n.d. | 46.6 ± 3.6 |
| Ginnalin B | 31.1 ± 1.9 | 22.6 ± 1.9 | n.d. | 47.1 ± 5.3 |
| Ginnalin C | 35.0 ± 1.4 | 29.8 ± 1.2 | n.d. | n.d. |

^{a}IC₅₀ (in μg/mL) is defined as the concentration required to achieve 50% inhibition over control cells (DMSO 0.5%); IC₅₀ values are shown as mean ± S.D. from three independent experiments; n.d. = not detected.

It should be noted that while ginnalin-A is indeed active, based on the IC₅₀ value of the most active extract (i.e. Red maple leaves containing 45% ginnalin A by weight) it is evident that the whole extract is superior to ginnalin A alone. Thus while ginnalin-A may be a major bioactive constituent, additive and/or synergistic effects among multiple constituents in the extract may impart greater biological effects beyond this compound alone. This is a common observation with botanical extracts and phytomedicines, whereby multiple constituents work synergistically to potentiate the activity of major active compounds.

Cell Cycle Distribution Analysis.

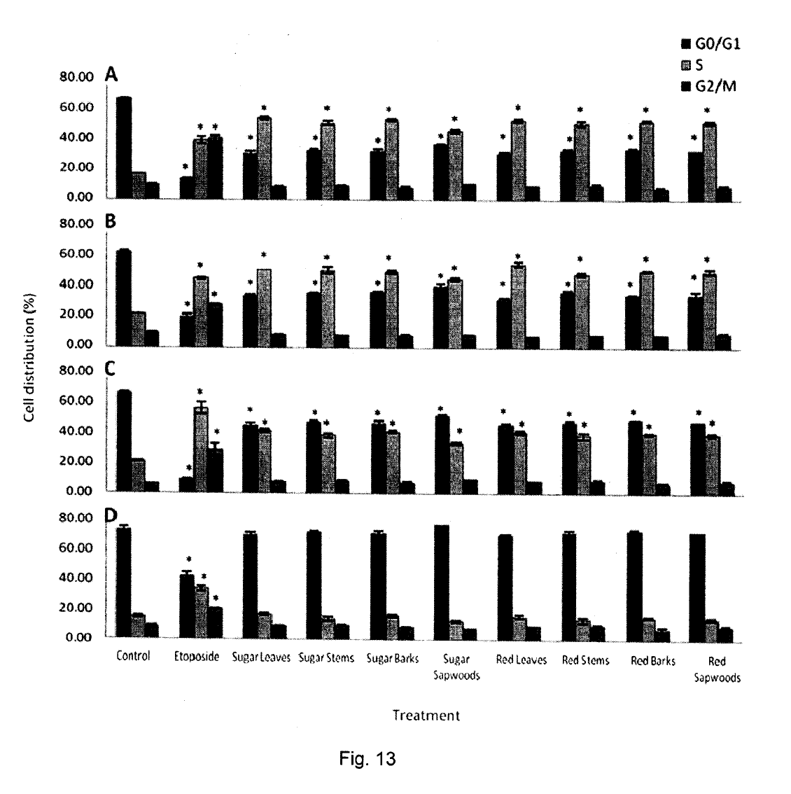

Inhibition of proliferation is further examined by measuring cell cycle distribution. At 48 h of the experiment, the HCT-116, Caco-2 and HT-29 control cells are distributed as follows: 58.7±3.6% in G₀/G₁ phase, 30.8±1.7% in S phase and 10.5±2.0% in G₂/M phase; 56.2±2.1% in G₀/G₁ phase, 31.0±2.4% in S phase and 12.8±0.40% in G₂/M phase; and 59.0±1.1% in G₀/G₁ phase, 31.1±0.9% in S phase and 9.9±0.5% in G₂/M phase, respectively (data not shown). At 72 h of the experiment, the proportion of these control cells in the G₀/G₁ phase increased to 66.3-70.9% whereas cells in the S and G₂/M phases decreased to 18.2-23.2% and to 7.2-9.7%, respectively (FIGS. 13A-C), indicating that there are no detectable effects of each cell line on cell cycle distribution.

At 48 h treatment with the maple plant part extracts (at doses corresponding to their IC₅₀ values) an increase of cells in S phase (p<0.05) concomitant with a decrease in G₀/G₁<0.05) and a slight increase in G₂/M phase are observed. In accordance with the HCT-116 cells being most sensitive among the cell lines in terms of reduced cell growth, changes observed in cell cycle distribution are more pronounced in these HCT-116 cells, with a clear arrest in the S-phase with a range of 45.8-55% (p<0.05). This increase is maintained during the 72 h of sample treatment to 48.6-57.3% (p<0.05), a 150% increase when compared to control cells in the S phase accompanied by a decrease of cells in G₀/G₁ phase (range 34.6-42.2%) (p<0.05) whereas no significant changes of the G₂/M ratio are observed (FIG. 13A). A similar trend is observed in the Caco-2 and HT-29 colon cancer cells treated with the maple extracts with 84 and 118% increases, and 72 and 96% increases, in the S arrest at 48 and 72 h, respectively (FIGS. 13B and 13C)

Notably, incubation of the normal colon CCD-18Co cells with the various maple plant part extracts for 48 and 72 h did not cause significant changes in cell cycle when compared with control cells (69.3±1.1% in G₀/G₁ phase, 17.6±0.9% in S phase and 13.1±1.0% in G₂/M phase; 76.5±2.0% in G₀/G₁ phase, 15.2±0.9% in S phase and 8.3±1.1% in G₂/M phase, respectively), except with the incubation of etoposide (50 μM) used as a positive control (FIG. 13D). These results indicated that the compounds present in the maple plant part extracts, at subtoxic levels, can inhibit the proliferation of colon cancer cells by blocking the progression of cell cycle at S-phase. Similarly, the inhibition of cell proliferation through cell cycle modulation has been described with other plant extracts on human colon and other cancer cell lines.

Apoptosis Assessment.

Another possible mechanism related to the antiproliferative activity of the maple plant part extracts in the colon cancer cells could be through the induction of apoptosis. Therefore, we carried out the morphological evaluation of apoptosis by monitoring for changes in nuclear chromatin distribution that can be stained by the DNA-binding fluorochrome Hoechst 33242 dye. Incubation of the colon cancer cells and normal colon cells with extracts mirrored the pattern followed by untreated cells, thus indicating the absence of apoptosis (data not shown). In contrast with our data, the hot water extract of the bark of Nikko maple (Acer nikoense) showed inhibitory effects on the growth of three murine cell lines by inducing cell death via apoptosis.

In conclusion, this is the first report of the evaluation of Sugar and Red maple species for their anticancer activity against human colon tumorigenic cells and investigation of their molecular mechanisms of action. The results indicate that the phenolic-enriched extracts of these maple species did not induce apoptosis but inhibited the proliferation of colon cancer cells due to cell cycle arrest in the S-phase. Moreover, the effects observed with the extracts are more pronounced on human colon cancer cells compared to the normal colon cells. The current results suggests that these maple plant extracts may have anti-colon cancer potential. Also, given that several chemotherapeutic agents have been isolated from plants, these maple (Acer) species may serve as promising candidates to yield potentially active antitumor compounds.

### Example 5

### Anti-Diabetic Activity of Maple Syrup and Maple Leaves Polyphenol Extracts

The potential of maple syrup and maple leaves (from both sugar and red maple trees) extracts for phenolic antioxidant-mediated type-2 diabetes management is evaluated in vitro by measuring their α-glucosidase inhibitory activities.

α-Glucosidase Inhibition Assay

All samples are diluted and adjusted to the same phenolic content (3%) and appropriate dilutions are performed to study dose-dependency. Briefly, a mixture of 50 μL extract or acarbose solution and 100 μl of 0.1 M phosphate buffer (pH 6.9) containing α-glucosidase solution (1.0 U/ml) was incubated in 96 well plates at 25° C. for 10 min. After pre-incubation, 50 μl of 5 mM p-nitrophenyl-α-D-glucopyranoside solution in 0.1 M phosphate buffer (pH 6.9) is added to each well at timed intervals. The reaction mixtures are incubated at 25° C. for 5 min. Before and after incubation, absorbance is recorded at 405 nm by micro-plate reader (VMax, Molecular Device Co., Sunnyvale, Calif., USA) and compared to that of the control which had 50 μL buffer solution in place of the extract. The α-glucosidase inhibitory activity is expressed as inhibition % and is calculated as follows:

$$\% inhibition = ( \frac{Δ {Abs}_{control} - Δ {Abs}_{sample}}{Δ {Abs}_{control}} ) \times 100$$

The inhibitory results are expressed as the half maximal inhibitory concentration (IC₅₀) which is a measure of the effectiveness of a compound in inhibiting biological or biochemical function

Statistical Analysis

All experiments were performed twice and analysis for each experiment is carried out in triplicates. Means, standard deviations and Pearson Product Moment Correlation Coefficient (PMCC−r) are determined using Microsoft Excel XP. IC₅₀ values are calculated using ED50plus vol. 1 developed by Vargas.

TABLE 18
IC₅₀ (μg solids) of sample syrup and maple leaves
and correlation with phenolic content

| Sample | IC50 (μg solids) | Phenolic content (%) | PMCC (r) |
|---|---|---|---|
| Sugar Maple Leaves | 13.15 | 35 | −0.96 |
| (MeOH) | | | |
| Red Maple Leaves | 36.03 | 45 | |
| (MeOH) | | | |
| Maple Syrup (MtOAc) | 318.36 | 34 | |
| Maple Syrup (BuOH) | 2,279.43 | 3 | |
| Maple Syrup (MeOH) | 1,389.72 | 9.6 | |

TABLE 19
IC₅₀ (μg phenolics) of maple syrup and maple leaves

| Sample | IC₅₀ (μg solids) |
|---|---|
| Sugar Maple Leaves (MeOH) | 4.66 |
| Red Maple Leaves (MeOH) | 16.23 |
| Maple Syrup (MtOAc) | 107.9 |
| Maple Syrup (BuOH) | 68.38 |
| Maple Syrup (MeOH) | 133.44 |

The leaf extracts have higher total phenolic content than the syrup extracts and among these, the red maple leaf methanol extract (RL-MeOH) has the highest total phenolic content (450 mg/g DW) followed by the sugar maple leaf methanol extract (SL-MeOH) (350 mg/g DW). The ethyl acetate extract of maple syrup (MS-EtOAc) (340 mg/g DW) has higher total phenolic content than the methanol (MS-MeOH) (96 mg/g DW) or butanol (MS-BuOH) (30 mg/g DW) extracts. The antioxidant activity in terms of DPPH free radical scavenging activity correlates with the observed total phenolic contents with RL-MeOH having the highest activity (IC₅₀ 6.48 ppm). All the tested extracts have α-glucosidase inhibitory activity. On a dry weight basis, the observed inhibitory activities correlated well (R=−0.96) with phenolic contents. SL-MeOH (IC₅₀ 13.15 μg) had higher inhibitory activity than RL-MeOH (IC₅₀ 36.03 μg), while MS-BuOH has the lowest (IC₅₀ 2,279.43 μg). On a phenolic content basis, SL-MeOH has the highest inhibitory effect (IC₅₀ 4.66 μg phenolic) followed by RL (IC₅₀ 16.23 μg phenolic). For the syrup extracts, MS-BuOH has higher inhibitory activity (IC₅₀ 68.38 μg phenolic) followed by MS-EtOAc and MS-MeOH with IC₅₀ values of 107.9 and 133.44 μg phenolic, respectively. These results suggest that both maple leaves and maple syrup extracts have potential for type 2 diabetes management, metabolic syndrome management and their α-glucosidase inhibitory activities depend on the phenolic phytochemical profile.

### Example 6

### Anti-Inflammatory Activity of Maple Syrup Polyphenol Extracts

Inflammation and pro-inflammatory processes are implicated in several chronic human diseases including metabolic syndrome, diabetes, cardiovascular diseases, neurodegenerative diseases, oxidative stress related disease, inflammation and an inflammatory condition, intestinal dysfunction (Crown's disease, inflammatory bowel diseases, etc) and cancer. The release of pro-inflammatory mediators including nitric oxide (NO) and prostaglandin-E2 (PGE-2) have been associated with inflammatory conditions, through the activity of their inducible enzymes, inducible nitric oxide synthase (iNOS) and cyclooxygenase-2 (COX-2) respectively, via the nuclear factor kappa B (NF-κB) signaling pathway. Overwhelming data suggests that dietary polyphenols, a large class of bioactive plant natural products, show anti-inflammatory properties.

The anti-inflammatory effects of a standardized polyphenolic-enriched maple syrup ethyl acetate extract (MS-EtOAc) in an lipopolysaccharide (LPS)-stimulated murine macrophages RAW 264.7 cell culture system are evaluated.

Nitric Oxide Assay

RAW 264.7 cells are seeded in 96 well plates for 24 hours at a density of 1×10⁵ cells/100 μl. The cells are then co-treated with compound (Crude extracts: concentrations 10, 50 & 100 PPM, Pure compounds: concentrations 1, 25, 50 μM) and Lipopolysaccharide (concentration: 10 ng/ml). Resveratrol is used as a positive control. After 24 hours of incubation 100 μl of the cell supernatant is mixed with 100 μl of 1× Griess reagent and the absorbance is measured at 540 nm after 15 min.

TABLE 20
Inhibition of Nitric Oxide

| | % Inhibition of Nitric oxide | | |
|---|---|---|---|
| | 10 | 50 | 100 |
| Source | PPM | PPM | PPM |
| Red maple leaves * | 35.2 | 75.6 | 96.8 |
| Red maple stem | 6.5 | 43.3 | 66.8 |
| Grade D butanol no sugar syrup | 0.0 | 14.9 | 10.6 |
| Grade D ethyl acetate syrup | 8.2 | 56.4 | 91.1 |
| Grade C ethyl acetate syrup | 23.2 | 56.3 | 95.4 |
| Grade C XAD methanol syrup | 37.7 | 36.7 | 49.4 |
| 4-5 grade C butanol | 0 | 0 | 0 |
| 4-5 grade C butanol sugar free | 3.7 | 0.2 | 13.6 |
| 4-5 grade C butanol + ethyl acetate syrup | 5.4 | 11.4 | 17.6 |
| Sugarmaple bark methanol extract | 19.8 | 15.4 | 8.0 |
| Sugarmaple bark ethyl acetate extract | 7.4 | 15.1 | 27.4 |
| Sugarmaple bark butanol extract | 100 | 100 | 100 |

TABLE 21
Definition of extracts according to phenolic content.

| | Concentration | |
|---|---|---|
| | polyphenols | % |
| Source | (mg/mL) | polyphenols |
| Sugar maple leaves | 43.796 | 35.0368 |
| Red maple leaves | 56.628 | 45.3024 |
| Red maple stem | 63.729 | 50.9832 |
| Sugar maple stem | 54.57 | 43.656 |
| 4-5 grade C butanol | 7.8 | 6.24 |
| Grade C EtOAc | 42.625 | 34.1 |
| Grade D butanol (no sugar) | 1.3 | 1.04 |
| Grade D EtOAc | 37.95 | 30.36 |
| Red maple stem (campus) | 48.125 | 38.5 |
| butanol | | |
| Red maple stem (campus) EtOAc | 68.125 | 54.5 |
| Red maple stem (campus) | 49.975 | 39.98 |
| Methanol | | |
| Norway maple stem (campus) | 22.7 | 18.16 |

MS-EtOAc extracts, dose dependently inhibited the overproduction of NO at concentrations ranging from 10-100 μg/mL. The effects of MS-EtOAc extracts on iNOS and COX-2 gene and protein expression, PGE-2 production, and NF-κB translocation are currently being evaluated to aid in elucidating its potential mechanism of anti-inflammatory action.

### Example 7

### Assessment of Anti-Diabetic Activity of Maple Syrup Polyphenol Extracts

The objective of the current example was to evaluate the type-2 diabetes management potential, via inhibition of carbohydrate hydrolyzing enzymes, of phenolic-enriched extracts of maple syrup (namely, ethyl acetate and butanol) in which sugars were previously removed.

Materials and Methods

Maple syrup (grade C) was provided by the Federation of Maple Syrup Producers of Quebec (Canada). The syrup is kept frozen until extraction. All solvents are of either ACS or HPLC grade and are purchased from Wilkem Scientific (Pawtucket, R.I.). α-Amylase (porcine pancreatic, EC 3.2.1.1), α-glucosidase (yeast, EC 3.2.1.20) and rat intestinal powder are purchased from Sigma-Aldrich (St. Louis, Mo.). Unless otherwise specified, all other chemicals are purchased from Sigma-Aldrich.

Sample Preparation

Preparation of phenolic-enriched extracts of maple syrup is as described above.

Total Phenolics Assay

Total phenolic content is determined as described above.

Antioxidant Activity Assay

The antioxidant potentials of MS-EtOAC and MS-BuOH are determined on the basis of the ability to scavenge the DPPH radicals as described above.

Carbohydrate Hydrolysis Enzyme Inhibition Assays

Since phenolic phytochemicals have been shown to have α-glucosidase inhibitory activity, the extracts are standardized to phenolic content (3.75 mg/mL GAE) to be evaluated on the same basis.

Yeast α-Glucosidase Inhibition Assay

A mixture of 50 μL of extract and 100 μl of 0.1 M phosphate buffer (pH 6.9) containing yeast α-glucosidase solution (1.0 U/ml) is incubated in 96 well plates at 25° C. for 10 min. After pre-incubation, 50 μl of 5 mM p-nitrophenyl-α-D-glucopyranoside solution in 0.1 M phosphate buffer (pH 6.9) is added to each well at timed intervals. The reaction mixtures are incubated at 25° C. for 5 min. Before and after incubation, absorbance is recorded at 405 nm by a micro-plate reader (VMax, Molecular Device Co., Sunnyvale, Calif., USA) and compared to that of the control which had 50 μL buffer solution in place of the extract. The α-glucosidase inhibitory activity is expressed as inhibition % and is calculated as follows:

$$\% inhibition = ( \frac{Δ {Abs}_{control} - Δ {Abs}_{sample}}{Δ {Abs}_{control}} ) \times 100$$

Rat α-Glucosidase Inhibition Assay

To validate the yeast α-glucosidase inhibition results, the rat α-glucosidase assay is used with the fractions that resulted at the highest inhibition. A total of 1 g of rat-intestinal acetone powder is suspended in 10 mL of 0.9% saline, and the suspension is sonicated twelve times for 30 sec at 4° C. After centrifugation (10000×g, 30 min, 4° C.), the resulting supernatant is used for the assay. Sample solution (50 μL) and 0.1 M phosphate buffer (pH 6.9, 100 μL) containing α-glucosidase solution is incubated at 25° C. for 10 min. After preincubation, 5 mM p-nitrophenyl-α-D-glucopyranoside solution (50 μL) in 0.1 M phosphate buffer (pH 6.9) is added to each well at timed intervals. The reaction mixtures are incubated at 25° C. for 30 min and readings are recovered every 5 min. Before and after incubation, absorbance is read at 405 nm and compared to a control which had 50 μL of buffer solution in place of the extract by micro-plate reader. The α-glucosidase inhibitory activity is expressed as inhibition % and is calculated as follows:

$$\% inhibition = ( \frac{{ΔAbs}_{control} - {ΔAbs}_{sample}}{Δ {Abs}_{control}} ) \times 100$$

Porcine α-Amylase Inhibition Assay

A mixture of 50 μL of extract or acarbose and 50 μL 0.02 M sodium phosphate buffer (pH 6.9 with 0.006 M sodium chloride) containing α-amylase solution (13 U/ml) are incubated at 25° C. for 10 min using an 1.5 mL Eppendorf tube. After pre-incubation, 50 μL 1% soluble starch solution in 0.02 M sodium phosphate buffer (pH 6.9 with 0.006 M NaCl) is added to each well at timed intervals. The reaction mixtures are then incubated at 25° C. for 10 min followed by addition of 1 mL dinitrosalicylic acid color reagent. The test tubes are then placed in a boiling water bath for 10 min to stop the reaction and cooled to room temperature. The reaction mixture is then diluted with 1 mL distilled water and absorbance is read at 540 nm using a 96-well microplate reader.

$$\% inhibi t ion = ( \frac{{ΔAbs}_{control} - {ΔAbs}_{sample}}{Δ {Abs}_{control}} ) \times 100$$

Statistical Analysis

All experiments are performed twice and analysis for each experiment is carried out in triplicate. Means, standard deviations, the degree of significance (p<0.05—One way ANOVA and t-Test) are determined using Microsoft Excel XP. Inhibition concentration (IC₅₀) values are calculated using ED50plus vol. 1 developed by Vargas (http://www.softlookup.com/display.asp?id=2972, accessed May 2009).

Results

Total Phenolic Content and Antioxidant Activity

On a dry weight (DW) basis, the MS-EtOAc extract has the highest total phenolic content (340 mg/g DW) followed by the MS-BuOH (30 mg/g DW) extract (Table 22). Similarly, for the antioxidant activity as measured by the DPPH free-radical scavenging assay, the MS-EtOAc extract exhibits higher antioxidant activity (IC₅₀=77.5 ppm) compared to the MS-BuOH fractions (IC₅₀>1000 ppm) (Table 22).

TABLE 22
Total phenolic contents and DPPH free-radical scavenging
activity of phenolic-enriched maple syrup extracts.

| | | DPPH Free- |
|---|---|---|
| | Total Phenolic | Radical |
| | Content (mg/g | Scavenging |
| Samples | GAE DW) | Activity (IC₅₀) |
| MS-EtOAc | 340 | 75.5 ppm |
| MS-BuOH | 30 | >1,000 ppm |

When ethyl acetate is used as an extracting solvent of maple syrup, it results in a high recovery of phenolic compounds. This may explain the higher observed antioxidant activity of ethyl acetate compared to butanol extracts (Table 22). The ethyl acetate extract of maple syrup also contains a wide variety of phenolic phytochemicals including small phenolic compounds and flavonoids, predominantly as flavonols and flavanols. We observe that the butanol extract of maple syrup (MS-BuOH) contains predominantly lignans, coumarins, and a stilbene, along with several previously reported small phenolic compounds. Thus, similar to other food matrices, the utilization of different organic solvents for extraction of maple syrup yields extracts with differing phenolic profiles. While both MS-EtOAc and MS-BuOH contains predominantly phenolic compounds, their individual phenolic constituents are quite different.

Yeast/Rat α-Glucosidase and Porcine α-Amylase Inhibition Assay

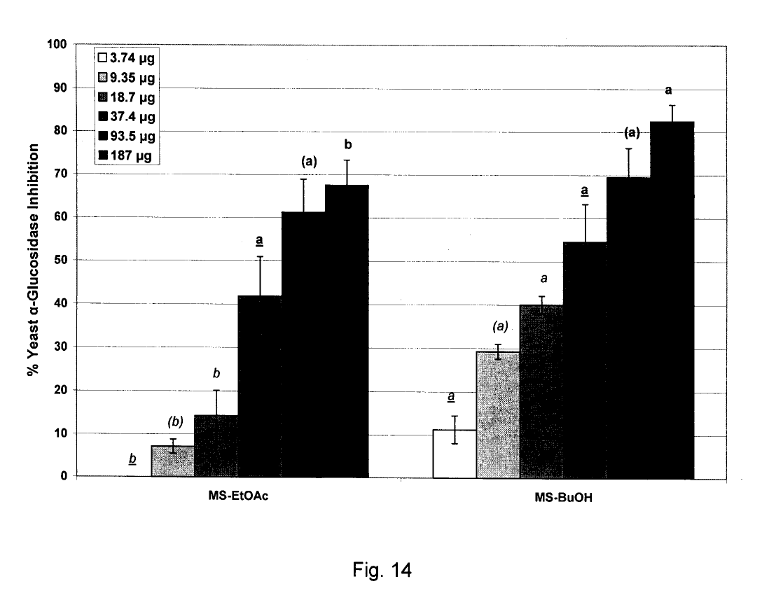

The extracts are standardized to phenolic content (3.75 mg/mL GAE) and assayed for yeast α-glucosidase inhibition. Both extracts have a dose-dependent α-glucosidase inhibitory activity with the MS-BuOH having the highest (82% at highest dose, IC₅₀ 68.38 μg phenolics) followed by MS-EtOAc (67% at highest dose, IC₅₀ 107.9 μg phenolics) (FIG. 14).

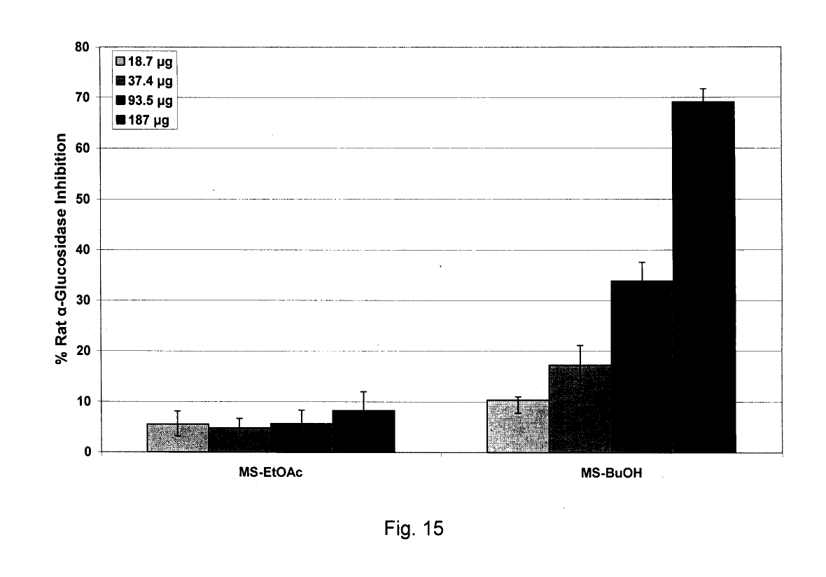

Yeast α-glucosidase assay can be an inexpensive and rapid method to screen for potential α-glucosidase inhibitors as done in the initial assays used herein. However, based on the observed inhibitory activities in the yeast α-glucosidase assay, we further evaluated MS-EtOAc and MS-BuOH for rat α-glucosidase inhibition. The results in the rat α-glucosidase assay show that MS-BuOH extract has a higher dose-dependent inhibitory activity than the MS-EtOAC extract (69% at the highest dose, IC₅₀ 135 μg phenolics and 8% at the highest dose, IC₅₀>187 μg phenolics, respectively) (FIG. 15). We note that the MS-EtOAC extract has almost no activity, since no dose-dependency was indicated and the observed results could be due to the limitation of the assay at very low inhibitory activities (FIG. 15).

The findings indicate that when the extracts are evaluated at equivalent phenolic content, the MS-BuOH exhibited higher α-glucosidase inhibition potential in the yeast based assay (FIG. 14). Similarly, when MS-EtOAC and MS-BuOH are further evaluated for rat α-glucosidase inhibition, it is clear that MS-BuOH fraction has higher potential for α-glucosidase inhibition in the rat-based assay (FIG. 15). These results suggest that the unique combination of phenolic phytochemicals present in the MS-BuOH extract may have higher potential for α-glucosidase inhibition.

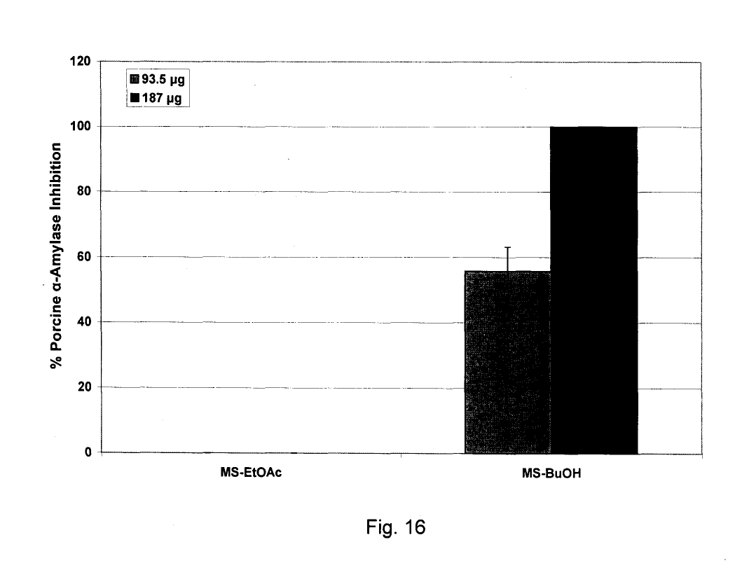

The phenolic standardized MS-BuOH and MS-EtOAc extracts are further assayed for α-amylase inhibition in a porcine based assay. At the test concentrations, the MS-EtOAc extract has no inhibitory activity (IC₅₀>187 μg) while the MS-BuOH extract has α-amylase inhibition with IC₅₀=103 μg phenolics (FIG. 16).

Previous reports have indicated that phenolic phytochemicals have lower α-amylase inhibitory activity and a stronger inhibition activity against yeast α-glucosidase. The MS-BuOH extract of maple syrup has significantly milder α-amylase inhibitory activity (FIG. 16) compared to its observed yeast α-glucosidase inhibitory activity (FIG. 14), however, it appears to have a rat α-glucosidase inhibitory activity at similar levels (FIG. 15). Optimum inhibition of both α-amylase and α-glucosidase enzymatic activities may result in slower oligosaccharide release from starch, with subsequent slower glucose absorption in the small intestine, thus better moderation of postprandial blood glucose increase.

Phenolic phytochemicals are secondary metabolites of plant origin which constitute one of the most abundant and ubiquitous groups of natural metabolites and form an important part of both human and animal diets. Recent studies have shown that phenolic phytochemicals have high antioxidant activity and other biological properties. The phenolic constituents of maple syrup in different extracts are further related to antioxidant, and human cancer cell antiproliferative anti-inflammatory properties. The present example shows that maple syrup phenolic-enriched extracts have type-2 diabetes management capability, via inhibition of carbohydrate hydrolyzing enzymes, with the MS-BuOH fraction having the highest bioactivity.

During the production of maple syrup, apart from natural phenolic constituents, other unique phenolic and non-phenolic compounds are formed during the intensive heating involved in transforming sap into syrup. Thus it is possible that these process-derived compounds may impart additional biological effects to maple syrup and may contribute to the observed health benefits and biological activities of maple syrup.

The present example shows the type-2 diabetes management potential of maple syrup and indicate that compared to MS-EtOAC, the MS-BuOH is the most active. The understanding of the mechanism of action and identification of compounds responsible for the observed α-glucosidase and α-amylase inhibitory activities coupled with animal and clinical trials could lead to the development of a maple syrup sweetener with lower glycemic index designed for type-2 diabetes management.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A molecule consisting of:
5-(3″,4″-dimethoxyphenyl)-3-hydroxy-3-(4′-hydroxy-3′-methoxybenzyl)-4-hydroxymethyl-dihydrofuran-2-one 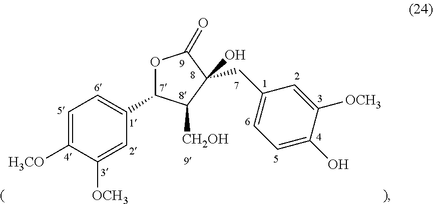 [SMILES: COc1cc(C[C@@]2(O)C(=O)O[C@@H](c3ccc(OC)c(OC)c3)[C@@H]2CO)ccc1O]
(erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol 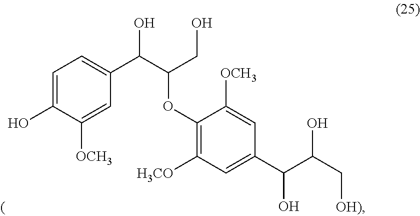
(erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol 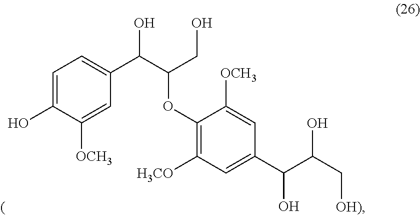
2,3-dihydroxy-1-(3,4-dihydroxyphenyl)-1-propanone 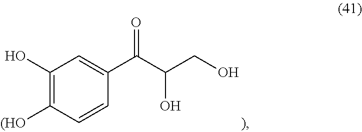
Quebecol

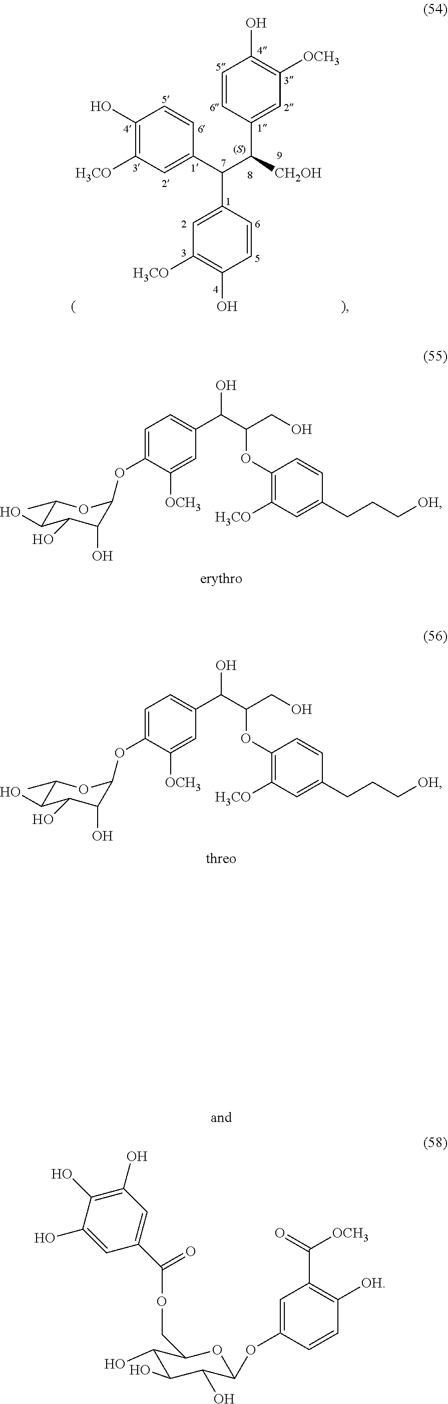

2. A phytochemical present in a maple tree butanol extract and methanol extract, which comprises a molecule chosen from:
Lyoniresinol,
Isolariciresinol,
secoisolariciresinol,
Dehydroconiferyl alcohol,
5′-methoxy-dehydroconiferyl alcohol,
erythro-guaiacylglycerol-β-O-4′-coniferyl alcohol,
erythro-guaiacylglycerol-β-O-4′-dihydroconiferyl alcohol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
5-(3″,4″-dimethoxyphenyl)-3-hydroxy-3-(4′-hydroxy-3′-methoxybenzyl)-4-hydroxymethyl-dihydrofuran-2-one,
Scopoletin,
Fraxetin,
Isofraxidin,
Syringic acid,
Ginnalin B,
Trimethyl gallic acid methyl ester
(E)-3,3′-dimethoxy-4,4′-dihydroxy stilbene,
p-coumaric acid,
Ferulic acid,
(E)-Coniferol,
Syringenin,
Dihydroconiferyl alcohol,
C-veratroylglycol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
3′,4′,5′-Trihydroxyacetophenone,
4-Acetylcatechol,
2,4,5-Trihydroxyacetophenone,
1-(2,3,4-trihydroxy-5-methylphenyl)-ethanone,
2-Hydroxy-3′,4′-dihydroxyacetophenone,
Vanillin,
Syringaldehyde,
Catechaldehyde,
3,4-Dihydroxy-2-methylbenzaldehyde,
Catechol,
Catechin,
Epicatechin,
Quebecol,
(erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo,erythro)-1-[4-[(2-hydroxy-2-(4-hydroxy-3-methoxyphenyl)-1-(hydroxymethyl)ethoxy]-3-methoxyphenyl]-1,2,3-propanetriol,
(threo,threo)-1-[4-[(2-hydroxy-2-(4-hydroxy-3-methoxyphenyl)-1-(hydroxymethyl)ethoxy]-3-methoxyphenyl]-1,2,3-propanetriol,
threo-guaiacylglycerol-β-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Syringaresinol,
Icariside E4,
Sakuraresinol,
1,2-diguaiacyl-1,3-propanediol
protocatechuic acid,
4-(dimethoxymethyl)-pyrocatechol,
Tyrosol,
4-hydroxycatechol, and
Phaseic acid.

3. The phytochemical according to claim 2, wherein said phytochemical is from said maple tree butanol extract, which comprises a molecule chosen from:
Lyoniresinol,
Secoisolariciresinol,
Dehydroconiferyl alcohol,
5′-methoxydehydroconiferyl alcohol,
(1,3-Propanediol, 1-(4-hydroxy-3-methoxyphenyl)-2-[4-[(1E)-3-hydroxy-1-propenyl]-2-methoxyphenoxy]-, (1R,2R)),
1-(4-hydroxy-3-methoxyphenyl)-2-[4-(3-hydroxypropyl)-2-methoxyphenoxy]-propane-1,3-diol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
Scopoletin,
Fraxetin,
(E)-3,3′-dimethoxy-4,4′-dihydroxystilbene,
2-hydroxy-3′,4′-dihydroxyacetophenone,
1-(2,3,4-trihydroxy-5-methylphenyl)-ethanone,
2,4,5-trihydroxyacetophenone,
Catechaldehyde,
Vanillin,
Syringaldehyde,
Gallic acid,
Trimethyl gallic acid methyl ester,
Syringic acid,
Syringenin,
(E)-coniferol,
C-veratroylglycol,
Catechol,
Quebecol,
Catechin, and
Epicatechin.

4. The phytochemical according to claim 2, wherein said phytochemical is from said maple tree methanol extract, which comprises a molecule chosen from:
Gallic acid,
(E)-3,3′-dimethoxy-4,4′-dihydroxy stilbene,
Syringic acid,
C-veratroylglycol,
1-(4-hydroxy-3-methoxyphenyl)-2-[4-(3-hydroxypropyl)-2-methoxyphenoxy]-propane-1,3-diol,
3-[(4-[(6-dexoy-α-L-mannopyranosyl)oxy]-3-methoxyphenyl)-5-(3,4-dimethoxyphenyl)dihydro-3-hydroxy-4-(hydroxymethyl)-2(3H)-furanone,
Lyoniresinol,
2-Hydroxy-3′,4′-dihydroxyacetophenone,
Syringenin,
Catechol,
Syringaldehyde,
Vanillin,
1,3-propanediol,1-(4-hydroxy-3-methoxyphenyl)-2-[4-[(1E)-3-hydroxy-1-propenyl]-2-methoxyphenoxy]-,(1R,2R),
2,3-dihydro-3-(hydroxymethyl)-2-(4-hydroxy-3-methoxyphenyl)-7-methoxy-5-benzofuranpropanol (dihydrodehydrodiconiferyl alcohol),
Ferulic acid,
Catechaldehyde,
Fraxetin,
(E)-coniferyl alcohol (coniferol),
Scopoletin,
1-(2,3,4-trihydroxy-5-methylphenyl)-ethanone,
p-coumaric acid,
Secoisolariciresinol,
Catechin,
Epicatechin,
3′,4′,5′-Trihydroxyacetophenone,
4-(dimethoxymethyl)-pyrocatechol,
4-acetylcatechol,
2,3-dihydroxy-1-(3,4-dihydroxyphenyl)-1-propanone,
Dihydroconiferyl alcohol,
Isofraxidin,
2,3-dihydroxy-1-(4-hydroxy-3,5-dimethoxyphenyl)-1-propanone,
Tyrosol,
3-hydroxy-1-(4-hydroxy-3,5-dimethoxyphenyl)propan-1-one,
Isolariciresinol,
5-(3″,4″-dimethoxyphenyl)-3-hydroxy-3-(4′-hydroxy-3′-methoxybenzyl)-4-hydroxymethyl-dihydrofuran-2-one,
Protocatechuic acid,
Threo-guaiacylglycerol-β-O-4′-dihydroconiferyl alcohol,
4-hydroxycatechol,
(erythro,erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
1,2-diguaiacyl-1,3-propanediol,
(threo,erythro) 1-[4-[(1R,2R)-2-hydroxy-2-(4-hydroxy-3-methoxyphenyl)-1-(hydroxymethyl)ethoxy]-3-methoxyphenyl]-1,2,3-propanetriol,
(threo,threo) 1-[4-[(1R,2R)-2-hydroxy-2-(4-hydroxy-3-methoxyphenyl)-1-(hydroxymethyl)ethoxy]-3-methoxyphenyl]-1,2,3-propanetriol,
Leptolepisol D,
Sakuraresinol,
(erythro,threo)-1-[4-[2-hydroxy-2-(4-hydroxy-3-methoxyphenyl)-1-(hydroxymethyl)ethoxy]-3,5-dimethoxyphenyl]-1,2,3-propanetriol,
Icariside E4,
Syringaresinol,
Acernikol,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
2-[4-[(2S,3R)-2,3-dihydro-3-(hydroxymethyl)-5-(3-hydroxy propyl)-7-methoxy-2-benzofuranyl]-2,6-dimethoxyphenoxy]-1-(4-hydroxy-3-methoxyphenyl)-1,3-propanediol, and
Buddenol E.

5. A composition comprising a molecule according to claim 1, and a carrier.

6. The composition according to claim 5, wherein said composition is a cosmeceutical composition, a cosmetic composition, a nutraceutical composition, a functional food, a food ingredient, an additive, a non-food ingredient, a cosmeto-food, a pharmaceutical, a food supplement, a natural health product, or combinations thereof.

7. A method to prevent micro-organism infection, kill or inhibit micro-organism or treat micro-organism infection in a subject, which comprises administering an anti micro-organism amount of a molecule of claim 1.

8. A method to prevent micro-organism infection, kill or inhibit bacteria or treat micro-organism infection in a subject, which comprises administering an anti micro-organism amount of at least one phytochemical of claim 2.

9. The method according to claim 7, wherein said micro-organism is chosen from a bacterial, a fungus, and combinations thereof

10-11. (canceled)

12. A method of treating a disease in a subject, which comprises administering a therapeutically effective amount of a molecule of claim 1.

13. A method of treating or preventing a disease in a subject, which comprises administering a therapeutically effective amount of a phytochemical of claim 2.

14. The method according to of claim 12, wherein said disease is chosen from a metabolic syndrome, a diabetes, a neurodegenerative disease, an oxidative stress related disease, an intestinal dysfunction, a heart disease, a cancer, an inflammation and an inflammatory condition.

15-22. (canceled)

23. The method according to claim 14, wherein said intestinal dysfunction is chosen from an inflammatory bowel disease, Crohn's disease, an ulcerative colitis, collagenous colitis, lymphocytic colitis, ischaemic colitis, diversion colitis, Behçet's disease, and indeterminate colitis.

24-32. (canceled)

33. The method according to claim 14, wherein said diabetes is type 2 diabetes.

34. The molecule of claim 1, consisting of:

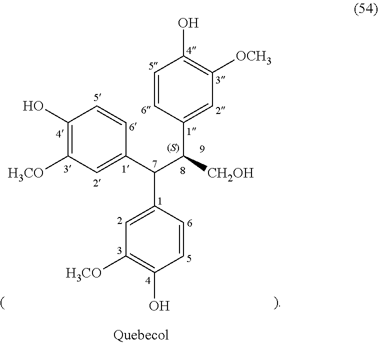

35. The composition of claim 5, wherein said molecule is

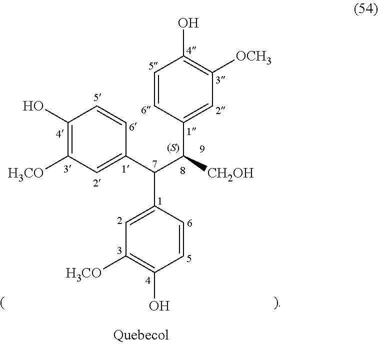

36. A composition comprising at least one phytochemical according to claim 2 and a carrier.

37. The method according to claim 13, wherein said disease is chosen from a metabolic syndrome, a diabetes, a neurodegenerative disease, an oxidative stress related disease, an intestinal dysfunction, a heart disease, a cancer, an inflammation and an inflammatory condition.

38. The method according to claim 37, wherein said diabetes is type 2 diabetes.
